(19) United States
(12) Patent Application Publication
(10) Pub. No.: US 2022/0380752 A1
(43) Pub. Date: Dec. 1, 2022

(54) DEVICES AND METHODS FOR EXTRACTING BLOOD PLASMA

(71) Applicant: Juno Diagnostics, Inc., San Diego, CA (US)

(72) Inventors: Dirk van den Boom, Encinitas, CA (US); Mathias Ehrich, San Diego, CA (US); Michael Van Nguyen, San Diego, CA (US); Christopher Ellison, La Jolla, CA (US); Jeffery William Domenighini, Encinitas, CA (US); Dylann Duncan Ceriani, San Diego, CA (US); Kristopher Scott Buchanan, Fort Collins, CO (US); Michael Gordon Smith, Fort Collins, CO (US)

(21) Appl. No.: 17/824,617

(22) Filed: May 25, 2022

Related U.S. Application Data
(60) Provisional application No. 63/193,442, filed on May 26, 2021.
(60) Provisional application No. 63/344,430, filed on May 20, 2022.

(51) Int. Cl. C12N 15/10; C12Q 1/6806

(52) U.S. Cl. CPC C12N 15/1017; C12N 15/1006; C12Q 1/6806

(57) ABSTRACT

Provided herein are devices and methods for extracting blood plasma from ultra-low volumes of blood. In some cases, the devices and methods use positive pressure to force a blood sample through a filter or a membrane. In some cases, the devices and methods use centrifugation to force a blood sample through a filter or a membrane. The devices and methods disclosed herein are suitable for the preparation of high-quality samples containing cell-free nucleic acids.

### CROSS-REFERENCE

This application claims the benefit of U.S. Provisional Application No. 63/193,442, filed on May 26, 2021, and U.S. Provisional Application No. 63/344,430, filed on May 20, 2022, each of which is incorporated herein by reference in its entirety.

### BACKGROUND

Traditional plasma separation from blood is generally performed by centrifugation. The blood is centrifuged in a primary spin to perform the bulk separation. In this initial spin, the majority of the cells are removed. The plasma remains on the top layer of the separated whole blood with the cells migrating to the bottom. The majority of the plasma is removed with the use of a pipette, but a small amount closest to the boundary layer between the plasma layer and cell layer is left behind as a precaution. Removing all of the plasma runs the risk of disturbing the cell layer which affects downstream processes. The plasma recovered from the primary spin is typically processed in a secondary spin to separate away any cells that may have been carried over after the primary spin. The final plasma recovery is removed using a pipette and, again, a small amount of plasma that is most likely to contain cells. In all, this process is time-consuming, requires special technical skills, and a substantial amount of plasma is lost in the process. Moreover, traditional plasma separation often leads to lysis of blood cells, thereby releasing cellular nucleic acids into the plasma. As such, plasma generated from traditional methods is often not suitable for use in downstream assays for detecting and/or analyzing cell-free nucleic acids.

### SUMMARY

There is an unmet need for devices and methods capable of efficiently extracting blood plasma from small volumes of blood. Additionally, there is an unmet need for methods and devices for efficiently enriching for cell-free nucleic acids from ultra-low volumes of blood. Moreover, there is an unmet need for methods and devices for generating high-quality samples containing cell-free nucleic acids that are suitable for downstream genetic analyses (e.g., such as samples that contain cell-free nucleic acids in an amount and quality suitable for downstream genetic analyses, e.g., that are not contaminated with cellular nucleic acids). This disclosure meets these unmet needs.

In one aspect, a method is provided comprising: (a) providing or obtaining a blood sample obtained from an individual; (b) applying a positive pressure to a starting volume of the blood sample such that the blood sample is pushed or forced into a filter or a membrane, wherein the starting volume of the blood sample is not more than about 1 milliliter (mL); (c) filtering the blood sample through the filter or the membrane to separate plasma from the blood sample, and (d) collecting the plasma in a collection vessel, wherein a volume of the plasma is greater than about 25% of the starting volume of the blood sample. In some cases, the volume of the plasma is greater than about 30%, greater than about 35%, or greater than about 40% of the starting volume of the blood sample. In some cases, at least about 60%, at least about 65%, at least about 70%, at least about 75%, at least about 80%, at least about 85%, at least about 90%, or greater, of the total amount of plasma present in the blood sample is collected. In some cases, the plasma comprises cell-free nucleic acids. In some cases, the method results in an enrichment of cell-free nucleic acids. In some cases, the starting volume of the blood sample is not more than about 500 microliters (μL), not more than about 250 μL, not more than about 150 μL, not more than about 100 μL, not more than about 80 μL, not more than about 60 μL, not more than about 50 μL, not more than about 40 μL, or not more than about 25 μL. In some cases, cellular nucleic acids are reduced in the plasma. In some cases, the plasma is substantially free of cellular nucleic acids. In some cases, cells, cell fragments, microvesicles, or any combination thereof, are reduced in the plasma. In some cases, the blood sample is or comprises capillary blood. In some cases, the blood sample is or comprises whole blood or one or more blood components. In some cases, a volume of plasma collected in (d) is greater than a volume of plasma collected using an equivalent method using negative pressure or vacuum. In some cases, a volume of plasma collected in (d) is at least about 10%, at least about 25%, at least about 50%, at least about 75%, at least about 100%, or greater than a volume of plasma collected using an equivalent method using negative pressure or vacuum. In some cases, the positive pressure is of an amount of less than about 4 psi, an amount of 4 psi to about 11 psi, or an amount greater than about 11 psi. In some cases, the method is performed in 1 minute or less. In some cases, the method does not result in substantial lysis or disruption of white blood cells. In some cases, the positive pressure is selected such that hemolysis of red blood cells may occur, but white blood cells are not lysed or disrupted. In some cases, the cell-free nucleic acids are deoxyribonucleic acid. In some cases, the cell-free nucleic acids comprise cell-free nucleic acids from a tumor. In some cases, the cell-free nucleic acids comprise cell-free nucleic acids from a fetus. In some cases, the cell-free nucleic acids comprise cell-free nucleic acids from a transplanted tissue or organ. In some cases, the cell-free nucleic acids comprise from about 10⁴ to about 10⁹ cell-free nucleic acid molecules. In some cases, the blood sample is obtained from the individual by a finger prick. In some cases, the method is performed on a point-of-care or point-of-need device.

In another aspect, a device is provided comprising: (a) a positive pressure source configured to exert a positive pressure on a blood sample and to push or force portions of the blood sample into a filter or membrane; and (b) the filter or membrane configured to separate plasma from the blood sample, wherein the device is configured to separate a volume of plasma from the blood sample that is greater than about 25% of an input volume of the blood sample. In some cases, the filter or membrane is configured to separate plasma from an input volume of no more than about 1 milliliter (mL) of the blood sample. In some cases, the filter or membrane is configured to separate plasma from an input volume of no more than about 500 microliters (μL) of the blood sample. In some cases, the filter or membrane is configured to separate plasma from an input volume of no more than about 100 microliters (μL) of the blood sample. In some cases, the filter or membrane is configured to separate plasma from an input volume of no more than about 50 microliters (μL) of the blood sample. In some cases, the volume of the plasma is greater than about 30%, greater than about 35%, or greater than about 40% of the starting volume of the blood sample. In some cases, at least about 60%, at least about 65%, at least about 70%, at least about 75%, at least about 80%, at least about 85%, at least about 90%, or greater, of the total amount of plasma present in the blood sample is collected. In some cases, the positive pressure source is a mechanical positive pressure source. In some cases, the positive pressure source comprises a material that possesses a spring force. In some cases, the positive pressure source comprises a foam material. In some cases, the device further comprises a structure configured to collect plasma from the filter or membrane and transport the plasma to a collection vessel, a collection region, or a collection structure within the device. In some cases, the device is a point-of-care or point-of-need device. In some cases, the filter or membrane is configured to remove or reduce a population of cells, cell fragments, microvesicles, or any combination thereof from the blood sample. In some cases, the device further comprises a sample inlet for introducing the blood sample into the device. In some cases, the sample inlet is configured to be sealed prior to exertion of the positive pressure on the blood sample. In some cases, the sample inlet is configured to be open during exertion of the positive pressure on the blood sample. In some cases, the filter or membrane comprises a plurality of pores. In some cases, a plurality of pores located at a first side of the filter or membrane have an average pore size that is greater than an average pore size of a plurality of pores located at a second side of the filter or membrane. In some cases, the device comprises two or more filters or membranes, each comprising a plurality of pores with an average pore size that is different for each filter or membrane.

In another aspect, a method is provided comprising: (a) providing or obtaining a blood sample obtained from an individual; (b) centrifuging the blood sample such that portions of the blood sample are forced or pushed through a filter or a membrane; (c) filtering the blood sample through the filter or the membrane to separate plasma from the blood sample; and (d) collecting the plasma in a collection vessel, wherein a volume of the plasma is greater than about 25% of an input volume of the blood sample. In some cases, the volume of the plasma is greater than about 30%, greater than about 35%, or greater than about 40% of the input volume of the blood sample. In some cases, the plasma comprises cell-free nucleic acids. In some cases, the method results in an enrichment of cell-free nucleic acids. In some cases, the input volume of the blood sample is not more than about 500 microliters (μL), not more than about 250 μL, not more than about 150 μL, not more than about 100 μL, not more than about 80 μL, not more than about 60 μL, not more than about 50 μL, not more than about 40 μL, or not more than about 25 μL. In some cases, cellular nucleic acids are reduced in the plasma. In some cases, the plasma is substantially free of cellular nucleic acids. In some cases, cells, cell fragments, microvesicles, or any combination thereof, are reduced in the plasma. In some cases, the blood sample is or comprises capillary blood. In some cases, the blood sample is or comprises whole blood or one or more blood components. In some cases, a volume of plasma collected in (d) is greater than a volume of plasma collected using an equivalent method using a centrifuge without the use of the filter or membrane. In some cases, a volume of plasma collected in (d) is at least about 10%, at least about 25%, at least about 50%, at least about 75%, at least about 100%, or greater than a volume of plasma collected using an equivalent method using a centrifuge without the use of the filter or membrane. In some cases, the method does not result in substantial lysis or disruption of white blood cells. In some cases, the cell-free nucleic acids are deoxyribonucleic acid. In some cases, the cell-free nucleic acids comprise cell-free nucleic acids from a tumor. In some cases, the cell-free nucleic acids comprise cell-free nucleic acids from a fetus. In some cases, the cell-free nucleic acids comprise cell-free nucleic acids from a transplanted tissue or organ. In some cases, the cell-free nucleic acids comprise from about 10⁴ to about 10⁹ cell-free nucleic acid molecules. In some cases, the whole blood sample is obtained from the individual by a finger prick. In some cases, the method is performed in a laboratory setting.

In yet another aspect, a device is provided comprising: (a) a sample inlet configured to introduce a blood sample into the device; (b) a filter or membrane configured to separate plasma from the blood sample; (c) a collection vessel configured to collect the plasma; and (d) an adapter, removably or permanently affixed to the device, configured to attach the device to a centrifuge. In some cases, the adapter is configured to attach the device to a centrifuge tube, to a plate, or interfaces to the centrifuge without an additional centrifuge tube. In some cases, the filter or membrane is configured to separate plasma from an input volume of no more than about 1 milliliter (mL) of the blood sample. In some cases, the filter or membrane is configured to separate plasma from an input volume of no more than about 500 microliters (μL) of the blood sample. In some cases, the filter or membrane is configured to separate plasma from an input volume of no more than about 100 microliters (μL) of the blood sample. In some cases, the filter or membrane is configured to separate plasma from an input volume of no more than about 50 microliters (μL) of the blood sample. In some cases, the filter or membrane is configured to separate plasma from an input volume of no more than about 25 microliters (μL) of the blood sample. In some cases, the filter or membrane is configured to remove or reduce a population of cells, cell fragments, microvesicles, or any combination thereof from the blood sample. In some cases, the filter or membrane comprises a plurality of pores. In some cases, a plurality of pores located at a first side of the filter or membrane have an average pore size that is greater than an average pore size of a plurality of pores located at a second side of the filter or membrane. In some cases, the device comprises two or more filters or membranes, each comprising a plurality of pores with an average pore size that is different for each filter or membrane.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the methods, devices, systems and kits disclosed herein are set forth with particularity in the appended claims. A better understanding of the features and advantages of the present devices, systems and kits disclosed herein will be obtained by reference to the following detailed description that sets forth illustrative embodiments, in which the principles of the devices, systems and kits disclosed herein are utilized, and the accompanying drawings of which:

FIG. 1 shows a non-limiting example of the components of a plasma separator device in which a positive pressure is applied to the component containing the membrane or filter by the rigid component, as described in some embodiments herein.

FIG. 2 shows an exploded view of a non-limiting example of a plasma separation device including a membrane or filter, a cartridge, a collection tube, and a rigid member, as described in some embodiments herein.

FIG. 3A and FIG. 3B show a semi-transparent isometric view and an exploded view of each individual layer of a component which contains the membrane or filter of a non-limiting example of a plasma separation device, as described in some embodiments herein.

FIGS. 4A-4D show a non-limiting example of a cartridge in various views including an exploded view detailing all sub-components of the cartridge, as described in some embodiments herein. FIG. 4D shows a cartridge with a membrane-containing component, as described in some embodiments herein.

FIGS. 5A-5C show a non-limiting example of the individual components of a rigid member, as described in some embodiments herein.

FIGS. 6A-6G show a non-limiting example of a manner by which the plasma separator device may be placed into the rigid member and mechanically displaced through the rigid member where the rigid member may supply sufficient positive pressure to the cartridge to extract plasma from a blood sample, as described in some embodiments here.

FIGS. 7A-7C show a non-limiting example of a membrane or filter interfacing with a blood collection tube placed into a corresponding cartridge, as described in some embodiments herein.

FIG. 8 depicts a non-limiting example of the components of a positive pressure plasma extractor, as described in some embodiments herein.

FIG. 9. depicts a non-limiting example of an exploded view of a positive pressure plasma extractor, as described in some embodiments herein.

FIG. 10 depicts a non-limiting example of a laminate stack, as described in some embodiments herein.

FIG. 11 depicts a non-limiting example of an exploded view of a laminate stack, as described in some embodiments herein.

FIG. 12 depicts a non-limiting example of a blood inlet layer, as described in some embodiments herein.

FIG. 13 depicts a non-limiting example of a blood metering layer, as described in some embodiments herein.

FIG. 14 depicts a non-limiting example of a blood metering layer with a preservative, as described in some embodiments herein.

FIG. 15 depicts a non-limiting example of a separation membrane layer, as described in some embodiments herein.

FIG. 16 depicts a non-limiting example of a membrane support layer, as described in some embodiments herein.

FIG. 17 depicts a non-limiting example of a membrane support layer with a preservative, as described in some embodiments herein.

FIG. 18 depicts a non-limiting example of a transfer channel layer, as described in some embodiments herein.

FIG. 19 depicts a non-limiting example of a lateral view of the laminate in the cartridge base, as described in some embodiments herein.

FIG. 20 depicts a non-limiting example of a laminate, a cartridge base, and a collection tube, as described in some embodiments herein.

FIG. 21 depicts a non-limiting example of a bottom view of the collection vial in a first position in the cartridge base (upper panel) and in the second position in the cartridge base (lower panel), as described in some embodiments herein.

FIG. 22 depicts a non-limiting example of a lateral view of a foil seal in the cartridge base, as described in some embodiments herein.

FIG. 23 depicts a non-limiting example of a lateral view of a slider, as described in some embodiments herein.

FIG. 24 depicts a non-limiting example of features of a slider, as described in some embodiments herein.

FIG. 25 depicts a non-limiting example of an overhead view (top panel) and a lateral view (bottom panel) of a plasma extractor in the open position, as described in some embodiments herein.

FIG. 26 depicts a non-limiting example of an overhead view (top panel) and a lateral view (bottom panel) of a plasma extractor in the closed position, as described in some embodiments herein.

FIG. 27 depicts a non-limiting example of a lateral view of a plasma extractor device in an open and closed position, as described in some embodiments herein.

FIG. 28A depicts a non-limiting example of a lateral view (top panel) and a cross section (bottom panel) of the device in an open position, as described in some embodiments herein.

FIG. 28B depicts a non-limiting example of a lateral view (top panel) and a cross section (bottom panel) of the device in a closed position, as described in some embodiments herein.

FIG. 29 depicts a non-limiting example of installing the shipping sleeve onto the cartridge base, as described in some embodiments herein.

FIG. 30 depicts a non-limiting example of a cross section of the shipping sleeve installation on the cartridge base, as described in some embodiments herein. The top panel depicts a partial installation of the shipping sleeve and the bottom panel depicts the full installation of the shipping sleeve.

FIG. 31 depicts a non-limiting example of a lateral view of a plasma extractor device, as described in some embodiments herein.

FIG. 32 depicts a non-limiting example of an interior view of a plasma extractor device, as described in some embodiments herein.

FIG. 33 depicts a non-limiting example of an exploded view of a plasma extractor device, as described in some embodiments herein.

FIG. 34 depicts a non-limiting example of a shipping container for a plasma extractor device, as described in some embodiments herein.

FIG. 35 depicts a non-limiting example of a laminate, as described in some embodiments herein.

FIG. 36 depicts a non-limiting example of an exploded view of the laminate, as described in some embodiments herein.

FIG. 37 depicts a non-limiting example of interactions between the enclosure base, laminate, and collection tube, as described in some embodiments herein.

FIG. 38 depicts a non-limiting example of a collection tube, as described in some embodiments herein.

FIG. 39 depicts a non-limiting example of a top view (top panel) and a bottom view (bottom panel) of the enclosure lid, as described in some embodiments herein.

FIG. 40 depicts a non-limiting example of a crush disc and an elastomer, as described in some embodiments herein.

FIG. 41 depicts a non-limiting example of interactions between an enclosure lid, a crush disc, and an elastomer in the open position (top panel) and the closed position (bottom panel), as described in some embodiments herein.

FIG. 42 depicts a non-limiting example of features of a slider, as described in some embodiments herein.

FIG. 43 depicts a non-limiting example of the interaction between the slider and the enclosure lid in the closed position (top panel) and open position (bottom panel), as described in some embodiments herein.

FIG. 44A depicts a non-limiting example of the interaction of the slider, crush disc, elastomer, and enclosure lid in the open position, as described in some embodiments herein.

FIG. 44B depicts a non-limiting example of a cross section of the slider, crush disc, elastomer, and enclosure lid in the closed position, as described in some embodiments herein.

FIG. 44C depicts a non-limiting example of a cross section of the slider, crush disc, and elastomer during actuation, as described in some embodiments herein.

FIG. 44D depicts a non-limiting example of a cross section of the slider, crush disc, elastomer, and enclosure lid during actuation, as described in some embodiments herein.

FIG. 45 depicts a non-limiting example of the features of the shipping container, as described in some embodiments herein.

FIG. 46 depicts a non-limiting example of the device in the open position, as described in some embodiments herein. The top panel is a lateral view and the bottom panel is a cross section.

FIG. 47 depicts a non-limiting example of the device in the closed position, as described in some embodiments herein. The top panel is a lateral view and the bottom panel is a cross section.

FIG. 48 depicts a non-limiting example of a lateral view of the device in the shipping container, as described in some embodiments herein.

FIG. 49 depicts a non-limiting example of cross sections of the shipping container being installed in the device, as described in some embodiments herein. The top panel shows the beginning of installation, the middle panel is partway through installation, and the bottom panel shows the shipping container fully installed on the device.

FIG. 50A and FIG. 50B show a non-limiting example of a centrifuge sample holder, as described in some embodiments herein.

FIG. 51A and FIG. 51B show a detailed structure of the membrane or filter layers of a non-limiting example of a plasma separator device, as described in some embodiments herein.

FIG. 52 shows a detailed exploded view of the membrane or filter layers of a non-limiting example of a plasma separator device capable of separating blood plasma in low volumes, as described in some embodiments herein.

FIG. 53 illustrates a workflow for a method of extracting blood plasma using a positive pressure blood plasma extractor, as described in some embodiments herein.

FIG. 54 illustrates a workflow for a method of extracting blood plasma using a centrifugation blood plasma extractor, as described in some embodiments herein.

FIGS. 55-58 depict a non-limiting example of the process of using a positive pressure plasma extractor, as described in some embodiments herein.

FIG. 59 depicts a non-limiting example of a lateral view of a positive pressure plasma extractor in an open position, as described in some embodiments herein.

FIG. 60 depicts a non-limiting example of a lateral view of a positive pressure plasma extract in a closed position, as described in some embodiments herein.

FIG. 61 depicts a non-limiting example of a lateral view of a positive pressure plasma extractor and a shipping case, as described in some embodiments herein.

FIG. 62 depicts a non-limiting example of a lateral view of a positive pressure plasma extractor inside a shipping case, as described in some embodiments herein.

FIGS. 63-65 show non-limiting examples of experimental results for the recovery of blood plasma from whole blood using the devices of the disclosure provided herein and the interactions between whole blood and plasma with Ethylenediaminetetraacetic acid (EDTA), as described in some embodiments herein.

FIG. 66 shows a non-limiting example of experimental data of a time course of Y chromosome-specific target sequence amplification via standard curve qPCR with ccfDNA extracted from blood either with or without a blood stabilization additive, as described in some embodiments herein.

FIG. 67 shows a non-limiting example of experimental data of temperature titration of Y chromosome-specific target sequence amplification via standard curve qPCR with ccfDNA extracted from blood either with or without a blood stabilization additive incubated at various temperatures for 24 hours, as described in some embodiments herein.

FIG. 68 shows a non-limiting example of experimental data of copy numbers measured from paired venous and capillary blood samples collected from pregnant women carrying a male fetus at time 0 (processed 2-4 hours after blood collection) and time 3 (processed 72 hours or 3 days post blood collection stored at ambient temperature), as described in some embodiments herein.

FIG. 69 shows a non-limiting example of experimental data of sequenced ccfDNA fragments from capillary collected maternal blood samples based on the ultralow input NIPT process, as described in some embodiments herein.

FIG. 70 shows a non-limiting example of experimental data of sequencing libraries obtained from capillary ccfDNA illustrating GC sequencing bias, as described in some embodiments herein.

FIG. 71 illustrates a non-limiting example of experimental data of the size distribution for capillary blood samples, as described in some embodiments herein.

FIG. 72 shows a non-limiting example of experimental data of chromosome Y and X sequencing bin counts, as described in some embodiments herein.

FIG. 73 shows a non-limiting example of experimental data of fetal fraction based on maternal plasma samples with male fetuses and clinical parameters including weight, body mass index (BMI), gestational age (GA) and age, as described in some embodiments herein.

FIG. 74 shows a non-limiting example of experimental data for chromosome 21 Z-scores for 62 subject samples based on ultralow input NIPT process starting from 20 μL of capillary blood (10 μL of plasma), as described in some embodiments herein.

FIG. 75 shows a non-limiting example of experimental data of Z-score classification of common trisomies 13, 18, and 21, as described in some embodiments herein.

FIG. 76 shows a non-limiting example of genome-wide, normalized bin counts between three study collection sites for capillary blood, as described in some embodiments herein.

FIG. 77 shows a non-limiting example of simulations of the sensitivity for detecting fetal trisomies at different genome equivalents (GE) inputs assuming different process efficiencies for an ultralow input NIPT workflow, as described in some embodiments herein.

FIG. 78 shows a non-limiting example of experimental data of the size distribution and size ratio comparison between capillary and venous blood, as described in some embodiments herein.

FIG. 79 shows a non-limiting example of experimental data of chromosome Y and chromosome X representations, as described in some embodiments herein.

FIG. 80 shows a non-limiting example of comparative fetal ccfDNA copy numbers in capillary versus venous collected blood as measured by standard curve qPCR in 50 euploid samples with known fetal size of which 22 are male, as described in some embodiments herein.

FIG. 81 shows a non-limiting example of experimental data of a comparison of z-scores between paired capillary and venous samples, as described in some embodiments herein.

FIG. 82 shows a non-limiting example of experimental data of Z-score classification of common trisomies 13, 18, and 31, as described in some embodiments herein.

FIG. 83 shows a non-limiting example of experimental data of a comparison of genome-wide, normalized bin counts between a paired capillary and venous sample which was positive for trisomy 21, as described in some embodiments herein.

FIGS. 84A-84C show a non-limiting example of training set results for ConVNet21, as described in some embodiments herein.

FIGS. 85A-85C show a non-limiting example of training set results for ConVNet13, as described in some embodiments herein.

FIGS. 86A-86C show a non-limiting example of training set results for ConVNet18, as described in some embodiments herein.

FIGS. 87A-87C show a non-limiting example of experimental data of the relationship between Z-score obtained in the Clinical Performance Verification Study and P-scores derived from the ConVNet prediction for test samples, as described in some embodiments herein.

FIG. 88 shows a non-limiting example of experimental data of a density plot of bin value distributions for the 10,000 bins in the probability tensor, as described in some embodiments herein.

FIGS. 89A-89C show a non-limiting example of experimental data of the relationship between Z-score obtained in the Clinical Performance Verification Study and P-scores derived from the ConVNet prediction for the validation set samples, as described in some embodiments herein.

FIG. 90 shows a non-limiting example of experimental data of a density plot of bin values distributions for the 10,000 bins in the probability tensor, as described in some embodiments herein.

FIG. 91 and FIG. 92 illustrate a non-limiting example of the architecture of ConVNet model for the detection of fetal trisomy, as described in some embodiments herein.

FIG. 93 illustrates a non-limiting example of the clinical study cohort of over 1200 samples divided into two sets of samples to assess performance of the ultralow input NIPT process including a training set of 671 samples and a test set of 550 samples, as described in some embodiments herein.

FIG. 94A and FIG. 94B show a non-limiting example of experimental data of sequenced ccfDNA fragments and fetal fraction as measured from the training and test stats, as described in some embodiments herein.

FIG. 95 shows a non-limiting example of confusion tables and sample plots for the common trisomies from the training set of samples, as described in some embodiments herein.

FIG. 96 shows a non-limiting example of confusion and summary table SCA performance of the training set, as described in some embodiments herein.

FIGS. 97A-97C show a non-limiting example of experimental data plots of the fetal sex chromosome aneuploidies samples from a study, as described in some embodiments herein.

FIG. 98 shows a non-limiting example of experimental data plots depicting multifetal samples in the study for the three common trisomies, as described in some embodiments herein.

FIG. 99 shows a non-limiting example of confusion tables and sample plots for common trisomies, and fetal sex calls from the test and training set, respectively, as described in some embodiments herein.

FIG. 100 illustrates a non-limiting example of experimental data of the classification of the less common fetal trisomies 16 and 22, as described in some embodiments herein.

FIG. 101 shows a non-limiting example of experimental data of the detection of fetal microdeletions, as described in some embodiments herein.

### DETAILED DESCRIPTION

### Certain Terminologies

The following descriptions are provided to aid the understanding of the methods, systems and kits disclosed herein. The following descriptions of terms used herein are not intended to be limiting definitions of these terms. These terms are further described and exemplified throughout the present application.

In general, the terms “cell-free polynucleotide”, and “cell-free nucleic acid”, used interchangeably herein, refer to polynucleotides and nucleic acids that can be isolated from a sample without extracting the polynucleotide or nucleic acid from a cell. A cell-free nucleic acid is a nucleic acid that is not contained within a cell membrane, e.g., it is not encapsulated in a cellular compartment. In some embodiments, a cell-free nucleic acid is a nucleic acid that is not bounded by a cell membrane and is circulating or present in blood or other fluid. In some embodiments, the cell-free nucleic acid is cell-free before and/or upon collection of the biological sample containing it, and is not released from the cell as a result of sample manipulation by man, intentional or otherwise, including manipulation upon or after collection of the sample. In some instances, cell-free nucleic acids are produced in a cell and released from the cell by physiological means, including, e.g., apoptosis, and non-apoptotic cell death, necrosis, autophagy, spontaneous release (e.g., of a DNA/RNA-lipoprotein complex), secretion, and/or mitotic catastrophe. In some embodiments, a cell-free nucleic acid comprises a nucleic acid that is released from a cell by a biological mechanism (e.g., apoptosis, cell secretion, vesicular release). In further or additional embodiments, a cell-free nucleic acid is not a nucleic acid that has been extracted from a cell by human manipulation of the cell or by sample processing (e.g., cell membrane disruption, lysis, vortex, shearing, etc.). The cell-free nucleic acid can comprise DNA, RNA, or both. In some cases, the cell-free nucleic acids may include DNA, RNA, microRNA (miRNA), long non-coding RNA (lncRNA), fetal DNA/RNA, mitochondrial DNA/RNA, or any combination thereof.

In some instances, the cell-free nucleic acid is a cell-free fetal nucleic acid. In general, the term, “cell-free fetal nucleic acid,” as used herein, refers to a cell-free nucleic acid, as described herein (e.g., that originates from the placenta (e.g., from placental trophoblasts) of a pregnant female). In some embodiments, the cell-free fetal nucleic acid is from a cell that comprises fetal DNA (e.g., placental trophoblasts).

Cell-free nucleic acids often originate from various different tissue types and are released into the circulation of an individual. Therefore, the pool of cell-free nucleic acids in circulation often represents the genetic makeup of the contributing tissue types. In the case of a healthy young individual, it can be a very homogenous pool without much variation. However, when a tissue contains a noticeably different genome, a more heterogeneous cell-free nucleic acid pool is often observed. Common examples include, but are not limited to: (a) cancer patients, where the tumor DNA contains mutated sites; (b) transplant patients, where the transplanted organ releases donor DNA into the pool of cell-free DNA; and (c) pregnant women, where the placenta contributes cell-free DNA that is largely representative of the fetal DNA.

In pregnant women, the cell-free DNA originating from the placenta can contribute a noticeable portion of the total amount of cell-free DNA. Placental DNA is often a good surrogate for the fetal DNA, because in most cases it is highly similar to the DNA of the fetus. Applications like chorionic villus sampling have exploited this fact to establish diagnostic application.

Often, a large portion of cell-free fetal nucleic acids are found in maternal biological samples as a result of placental tissue being regularly shed during the pregnant subject's pregnancy. Often, many of the cells in the placental tissue shed are cells that contain fetal nucleic acids. Cells shed from the placenta release fetal nucleic acids. Thus, in some instances, cell-free fetal nucleic acids disclosed herein are nucleic acids released from a placental cell. The cell-free fetal nucleic acids can comprise DNA or RNA.

In some instances, cellular nucleic acids (as defined below) are intentionally or unintentionally released from cells by devices and methods disclosed herein. However, these are not considered “cell-free nucleic acids,” as the term is used herein. In some instances, devices, systems, kits, and methods disclosed herein provide for analyzing cell-free nucleic acids in biological samples, and in the process analyze cellular nucleic acids as well.

As used herein, the term “cellular nucleic acid” refers to a polynucleotide that is contained in a cell or released from a cell due to manipulation of the biological sample. Non-limiting examples of manipulation of the biological sample include centrifuging, vortexing, shearing, mixing, lysing, and adding a reagent (e.g., detergent, buffer, salt, enzyme) to the biological sample that is not present in the biological sample when it is obtained. In some instances, the cellular nucleic acid is a nucleic acid that has been released from a cell due to disruption or lysis of the cell by a machine, human or robot. In some instances, the cellular nucleic acids is a nucleic acid that has been released from a cell due to non-physiological means (e.g., by a means other than apoptosis, non-apoptotic cell death, necrosis, autophagy, spontaneous release (e.g., of a DNA/RNA-lipoprotein complex), secretion, and/or mitotic catastrophe). The term “cellular nucleic acids” is not intended to encompass cell-free nucleic acids, as described herein.

In some cases, the methods and devices disclosed herein result in a reduction or depletion of cellular nucleic acids in a sample (e.g., plasma separated and collected using the methods and devices provided herein). In some cases, the methods and devices disclosed herein result in a sample (e.g., plasma separated and collected using the methods and devices provided herein) that is free or substantially free of cellular nucleic acids. The term “substantially free” as used in reference to cellular nucleic acids means that the sample does not contain any cellular nucleic acids, contains trace or minute amounts of cellular nucleic acids that are undetectable by standard methods (e.g., polymerase chain reaction, sequencing, and the like), or contains small amounts of cellular nucleic acids that do not interfere with downstream analysis of cell-free nucleic acids. In some cases, the methods and devices disclosed herein do not result in disruption or lysis of cells such that cellular nucleic acids are not released into the sample. In some cases, the methods or devices disclosed herein may reduce cellular nucleic acids in the plasma. By reducing cellular nucleic acids (e.g., by reducing cells contaminating the plasma or by reducing cell rupture leading to the release of cellular nucleic acids into the plasma), the methods and devices disclosed herein enrich for cell-free nucleic acids and specific components of interest (e.g., target cell-free nucleic acids) in the cell-free nucleic acids. By way of example, by reducing white blood cell-derived cellular nucleic acids, the relative amount of cell-free fetal DNA in prenatal testing is increased over conventional methods (e.g., venous blood draws). By way of another example, by reducing white blood cell-derived cellular nucleic acids, circulating tumor DNA is enriched (e.g., for oncology related applications).

The term “blood sample” as used herein refers to a sample (e.g., a liquid sample) that contains blood or one or more components of blood. In some cases, a blood sample is or comprises whole blood. In some cases, a blood sample is or comprises one or more blood components (e.g., plasma, white blood cells, red blood cells, etc.). In some cases, a blood sample contains additional components, such as, but not limited to, anti-coagulants, buffers, diluents, and the like.

The terms “recovered plasma”, “collected plasma”, or “expressed plasma”, are used interchangeably herein and generally refer to the plasma collected (e.g., from a starting sample) using the methods and devices provided herein. For example, the amount of recovered plasma or the amount of collected plasma or the amount of expressed plasma is the total amount of plasma that can theoretically be separated from a blood sample minus the amount of plasma that is lost during the separation process.

The terms “recoverable plasma”, “total plasma”, or “available plasma”, are used interchangeably herein and generally refer to the theoretical volume of plasma contained in a blood sample. The amount of recoverable plasma and the amount of recovered plasma generally differ as the entire volume of recoverable plasma is typically not recovered. In particular instances, the methods and devices disclosed herein generate a larger amount of recovered plasma (a higher percentage of recoverable plasma) versus standard plasma separation methods and devices.

As used herein, the term “about” a number refers to that number plus or minus 10% of that number. The term “about” when used in the context of a range refers to that range minus 10% of its lowest value and plus 10% of its greatest value.

As used herein, the singular forms “a”, “an”, and “the” include plural references unless the context clearly dictates otherwise. For example, the term “a sample” includes a plurality of samples, including mixtures thereof.

Traditional devices and methods generally cause the disruption of white blood cells, which release cellular nucleic acids into the plasma, thereby contaminating cell-free nucleic acid-enriched samples. Such devices and methods are therefore not suitable for extracting blood plasma for downstream genetic analysis of cell-free nucleic acids. This disclosure provides devices and methods for extracting plasma from a blood sample that does not have contaminating cellular nucleic acids (e.g., from white blood cells). The enriched cell-free nucleic acid sample is suitable for use with downstream genetic analyses.

In some aspects, methods and devices for separating and collecting plasma from a blood sample are provided. The methods of the disclosure may be performed on a point-of-care or point-of-need device as described herein. The method of the disclosure may be performed on a device used in a laboratory setting. In various aspects, the methods and devices described herein may be used to enrich for cell-free nucleic acids present in a blood sample. Advantageously, the methods and devices described herein are capable of collecting or recovering a substantial amount of plasma (e.g., recovered plasma) from an initial blood sample, without disrupting or lysing cells (e.g., white blood cells). Although the methods and devices may be used to separate and collect plasma for any downstream application, the methods and devices provided herein are especially suited for cell-free nucleic acid applications because the methods and devices result in high recovery amounts of plasma (containing cell-free nucleic acids) from ultra-low volumes of blood samples, without contaminating the plasma sample with cellular nucleic acids. Generally, the devices and provided herein are easy to use and do not require specialized technical skills. In some cases, the devices may be point-of-care or point-of-need devices that may be used in a healthcare setting or a home setting.

In some cases, devices and methods disclosed herein allow for diagnosing and/or monitoring medical conditions. Non-limiting examples of medical conditions include autoimmune conditions, metabolic conditions, cancer, and neurological conditions. Devices and methods disclosed herein allow for personalized medicine, including microbiome testing, determining an appropriate personal medical dosage, and/or detecting a response to a drug or dose thereof. Devices and methods disclosed herein also allow for detecting a food allergen and detecting food/water contamination. Devices and methods disclosed herein provide for detecting an infection by a pathogen and/or a subject's resistance to drugs that could be used to treat the infection. Generally, with the methods and devices disclosed herein, there is little to no need for technical training or large, expensive laboratory equipment.

### Devices

Aspects of the disclosure herein comprise devices configured to separate and collect blood plasma from blood samples. Generally, the devices provided herein comprise a membrane or filter configured to separate plasma from a blood sample (e.g., by allowing plasma to flow through the filter while capturing one or more non-plasma blood components). The devices provided herein advantageously allow for the separation of plasma from ultra-low volumes of blood and collect the plasma with high recovery rates. The devices provided herein further allow for enrichment of cell-free nucleic acids. The devices provided herein are gentle such that cells (e.g., white blood cells) are not disrupted or lysed, thereby contaminating the collected plasma with cellular nucleic acids. In one embodiment, the plasma separation device includes a positive pressure source that allows for high recovery rates of plasma and enrichment of cell-free nucleic acids. In another embodiment, the plasma separation device comprises a component configured to be used with a centrifuge (e.g., typically found in a laboratory setting). The devices provided herein overcome difficulties and challenges currently experienced in plasma separation and cell-free nucleic acid sample preparation methods.

Plasma Separation Devices with Positive Pressure Source

In some embodiments, the devices described herein may comprise a blood plasma separator wherein the blood plasma separator extracts blood plasma through the application of positive pressure. In various aspects, the device may comprise: (a) a positive pressure source configured to exert a positive pressure on a blood sample and to push the blood sample into a filter or membrane; and (b) the filter or membrane configured to separate plasma from the blood sample.

The device may be configured to separate and collect a volume of plasma from the blood sample that is greater than about 25% of an input or starting volume of the blood sample. For example, the device may be configured to separate a volume of plasma from the blood sample and collect a volume that is greater than about 30%, greater than about 35%, greater than about 40%, or more of an input or starting volume of the blood sample.

The device may be configured to collect an amount of plasma (e.g., recovered plasma) that is at least about 50% of the total plasma volume present in the blood sample (e.g., recoverable plasma). For example, the device may be configured to collect an amount of plasma (e.g., recovered plasma) that is at least about 55%, at least about 60%, at least about 65%, at least about 70%, at least about 75%, at least about 80%, at least about 85%, at least about 90%, at least about 95%, or greater of the total plasma volume present in the blood sample (e.g., recoverable plasma).

In some aspects, the device is configured to collect an amount of plasma (e.g., recovered plasma) that is at least about 60%, at least about 65%, at least about 70%, at least about 75%, at least about 80%, at least about 85%, at least about 90%, or greater of the total plasma volume present in 50 μL of whole blood (e.g., recoverable plasma).

The devices may be configured to separate and collect plasma from an ultra-low volume of a blood sample. In some instances, devices disclosed herein are used with not more than 25 μL of the blood sample. In some instances, devices disclosed herein are used with not more than 50 μL of the blood sample. In some instances, devices disclosed herein are used with not more than 75 μL of the blood sample. In some instances, devices disclosed herein are used with not more than 100 μL of the blood sample. In some instances, devices disclosed herein are used with not more than 125 μL of the blood sample. In some instances, devices disclosed herein are used with not more than 150 μL of the blood sample. In some instances, devices disclosed herein are used with not more than 200 μL of the blood sample. In some instances, devices disclosed herein are used with not more than 300 μL of the blood sample. In some instances, devices disclosed herein are used with not more than 400 μL of the biological fluid sample. In some instances, devices disclosed herein are used with not more than 500 μL of the biological fluid sample. In some instances, devices disclosed herein are used with not more than 1 mL of the biological fluid sample.

In some instances, the devices disclosed herein are used with an ultra-low volume of a blood sample, wherein the ultra-low volume falls within a range of sample volumes. In some instances, the range of sample volumes is about 5 μL to about 1 mL. In some instances, the range of sample volumes is about 5 μL to about 900 μL. In some instances, the range of sample volumes is about 5 μL to about 800 μL. In some instances, the range of sample volumes is about 5 μL to about 700 μL. In some instances, the range of sample volumes is about 5 μL to about 600 μL. In some instances, the range of sample volumes is about 5 μL to about 500 μL. In some instances, the range of sample volumes is about 5 μL to about 400 μL. In some instances, the range of sample volumes is about 5 μL to about 300 μL. In some instances, the range of sample volumes is about 5 μL to about 200 μL. In some instances, the range of sample volumes is about 5 μL to about 150 μL. In some instances, the range of sample volumes is 5 μL to about 100 μL. In some instances, the range of sample volumes is about 5 μL to about 90 μL. In some instances, the range of sample volumes is about 5 μL to about 85 μL. In some instances, the range of sample volumes is about 5 μL to about 80 μL. In some instances, the range of sample volumes is about 5 μL to about 75 μL. In some instances, the range of sample volumes is about 5 μL to about 70 μL. In some instances, the range of sample volumes is about 5 μL to about 65 μL. In some instances, the range of sample volumes is about 5 μL to about 60 μL. In some instances, the range of sample volumes is about 5 μL to about 55 μL. In some instances, the range of sample volumes is about 5 μL to about 50 μL. In some instances, the range of sample volumes is about 15 μLμμ to about 150 μL. In some instances, the range of sample volumes is about 15 μL to about 120 μL. In some instances, the range of sample volumes is 15 μL to about 100 μL. In some instances, the range of sample volumes is about 15 μL to about 90 μL. In some instances, the range of sample volumes is about 15 μL to about 85 μL. In some instances, the range of sample volumes is about 15 μL to about 80 μL. In some instances, the range of sample volumes is about 15 μL to about 75 μL. In some instances, the range of sample volumes is about 15 μL to about 70 μL. In some instances, the range of sample volumes is about 15 μL to about 65 μL. In some instances, the range of sample volumes is about 15 μL to about 60 μL. In some instances, the range of sample volumes is about 15 μL to about 55 μL. In some instances, the range of sample volumes is about 15 μL to about 50 μL.

In some instances, the devices disclosed herein are used with an ultra-low volume of a blood sample, wherein the ultra-low volume is about 100 μL to about 500 μL. In some instances, the devices disclosed herein are used with an ultra-low volume of the blood sample, wherein the ultra-low volume about 100 μL to about 1000 μL. In some instances, the ultra-low volume is about 500 μL to about 1 ml. In some instances, the ultra-low volume is about 500 μL to about 2 ml. In some instances, the ultra-low volume is about 500 μL to about 3 ml. In some instances, the ultra-low volume is about 500 μL to about 5 ml.

The ultra-low volume may be about 1 μL to about 250 μL. The ultra-low volume may be about 5 μL to about 250 μL. The ultra-low volume may be about 10 μL to about 25 μL. The ultra-low volume may be about 10 μL to about 35 μL. The ultra-low volume may be about 10 μL to about 45 μL. The ultra-low volume may be about 10 μL to about 50 μL. The ultra-low volume may be about 10 μL to about 60 μL. The ultra-low volume may be about 10 μL to about 80 μL. The ultra-low volume may be about 10 μL to about 100 μL. The ultra-low volume may be about 10 μL to about 120 μL. The ultra-low volume may be about 10 μL to about 140 μL. The ultra-low volume may be about 10 μL to about 150 μL. The ultra-low volume may be about 10 μL to about 160 μL. The ultra-low volume may be about 10 μL to about 180 μL. The ultra-low volume may be about 10 μL to about 200 μL.

The ultra-low volume may be about 1 μL to about 200 μL. The ultra-low volume may be about 1 μL to about 190 μL. The ultra-low volume may be about 1 μL to about 180 μL. The ultra-low volume may be about 1 μL to about 160 μL. The ultra-low volume may be about 1 μL to about 150 μL. The ultra-low volume may be about 1 μL to about 140 μL. The ultra-low volume may be about 5 μL to about 15 μL. The ultra-low volume may be about 5 μL to about 25 μL. The ultra-low volume may be about 5 μL to about 35 μL. The ultra-low volume may be about 5 μL to about 45 μL. The ultra-low volume may be about 5 μL to about 50 μL. The ultra-low volume may be about 5 μL to about 60 μL. The ultra-low volume may be about 5 μL to about 70 μL. The ultra-low volume may be about 5 μL to about 80 μL. The ultra-low volume may be about 5 μL to about 90 μL. The ultra-low volume may be about 5 μL to about 100 μL. The ultra-low volume may be about 5 μL to about 125 μL. The ultra-low volume may be about 5 μL to about 150 μL. The ultra-low volume may be about 5 μL to about 175 μL. The ultra-low volume may be about 5 μL to about 200 μL.

In various aspects, a device of the disclosure may comprise a positive pressure source. It should be understood that any type of positive pressure source may be used to exert a positive pressure on a sample as described herein. In a particular embodiment, the positive pressure source may be a mechanical positive pressure source. In some cases, the mechanical positive pressure source may comprise a material (e.g., a foam) that possesses a spring-like force.

In various aspects, the positive pressure source is configured to exert a positive pressure on a blood sample. In some cases, the positive pressure source is configured to exert a positive pressure of about 4 psi to about 11 psi on the blood sample. In some cases, the positive pressure source is configured to exert a positive pressure of about 11 psi to about 20 psi on the blood sample. In some cases, the positive pressure source exerts a positive pressure of less than about 4 psi on the blood sample (e.g., less than 3 psi, less than 2 psi, less than 1 psi, or even less). In some cases, the positive pressure source exerts a positive pressure of greater than 11 psi on the blood sample (e.g., greater than 15 psi, greater than 20 psi, greater than 25 psi, or even greater). In some cases, the positive pressure source is configured to exert a positive pressure that is sufficient to force the blood sample through the filter or membrane without causing disruption (e.g., lysis, shearing) of cells (e.g., white blood cells). In some cases, the positive pressure source is configured to exert a positive pressure sufficient to enrich for cell-free nucleic acids without causing disruption of cells and release of contaminating cellular nucleic acids into the plasma. In some cases, the positive pressure source may exert a positive pressure that causes disruption of red blood cells (which do not contain cellular nucleic acids) but does not cause disruption of white blood cells.

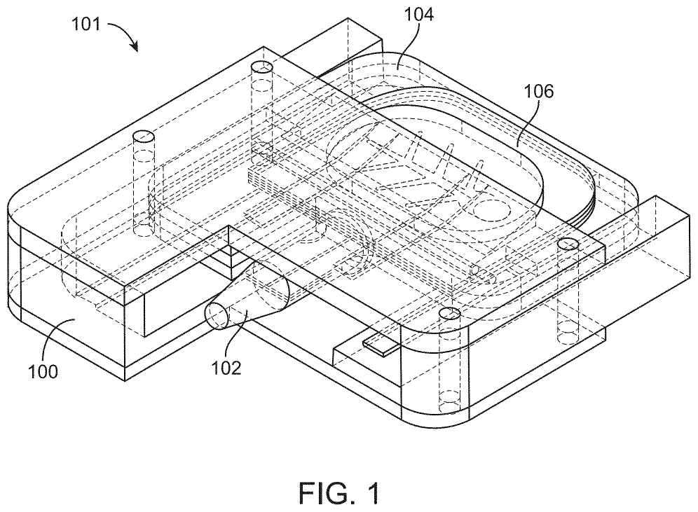

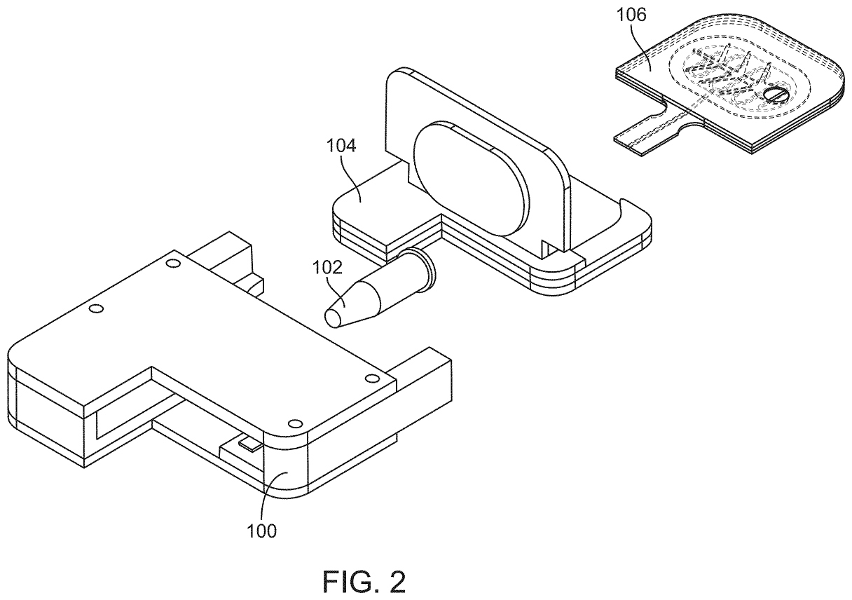

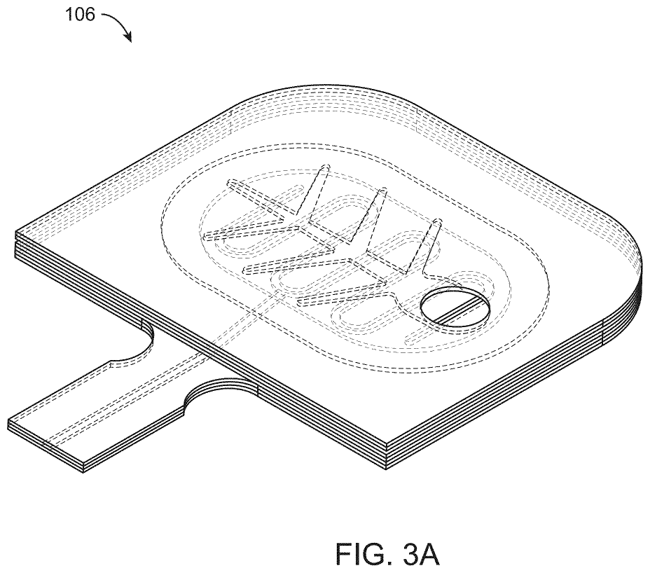

FIG. 1 depicts a non-limiting example of a plasma separation device of the disclosure that uses a positive pressure source. The device may comprise a positive pressure source (e.g., a mechanical positive pressure source) configured to exert a positive pressure on the blood sample. The device may further comprise a filter or membrane 304 (e.g., as shown in the laminate depicted in FIG. 3B) configured to separate plasma from the whole blood sample. In some instances, the device may comprise two or more filters or membranes, described elsewhere herein, each comprising a plurality of pores with an average pore size that is different for each filter or membrane. The device may further comprise one or more of a housing 100, a collection vessel 102, a cartridge 104, a laminate 106, or any combination thereof, e.g., as seen in FIG. 2. In some cases, the collection tube 102 may comprise an Eppendorf tube, PCR tube, or any combination thereof.

In some instances, the laminate 106, seen in FIG. 3A and FIG. 3B may be disposable. In some instances, the laminate may comprise a plurality of layers (300-312). In some cases, the laminate may comprise one or more of the following: a blood inlet layer 300; a blood metering layer 302; a membrane or filter layer 304; an adhesive layer 306; a membrane support layer 308; a transfer channel layer 310; and a base layer 312. In some cases, the blood inlet layer 300 may comprise an inlet configured to accept a blood sample. The inlet layer may provide protection to the other layers of the laminate (e.g., the filter or membrane). In some instances, the blood metering layer 302 may be configured to meter an input blood sample placed into the device. In some cases, when a volume of a blood sample is deposited that exceeds the capacity of the device, the blood metering layer may take up some of the blood sample and the remainder of the blood sample remains outside the inlet layer. In some cases, the membrane or filter layer 304 is configured to trap or block one or more blood components from passing through the filter. The membrane or filter may comprise a plurality of pores. In some instances, the plurality of pores located at a first side of the filter or membrane have an average pore size that is greater than an average pore size of a plurality of pores located at a second side of the filter or membrane 304. In one embodiment, the membrane or filter has larger pores on the surface closer to the blood inlet layer 300, and smaller pores on or near the surface closer to the transfer channel 310. Non-limiting examples of such filters include Pall Vivid™ GR membrane, Munktell Ahlstrom filter paper (see, e.g., WO2017017314), TeraPore filters. In an alternative embodiment, the device may comprise two or more membranes or filters, wherein each membrane or filter has a different average pore size. For example, a membrane or filter located closer to the blood inlet layer 300 may have a larger average pore size than a membrane or filter located closer to the transfer channel 310. In some cases, the filter or membrane layer is configured to remove or reduce a population of cells, cell fragments, microvesicles, or any combination thereof from the blood sample.

In some embodiments, the device may comprise a lateral filter (e.g., sample does not move in a gravitational direction or the sample moves perpendicular to a gravitational direction). In some embodiments, the device may comprise a vertical filter (e.g., sample moves in a gravitational direction). In some embodiments, the device may comprise both a vertical filter and a lateral filter. In some embodiments, the device may be configured to receive a sample or portion thereof with a vertical filter, followed by a lateral filter. In some embodiments, the device may be configured to receive a sample or portion thereof with a lateral filter, followed by a vertical filter. In some instances, a vertical filter comprises a filter matrix. In some instances, the filter matrix of the vertical filter comprises a pore with a pore size that is prohibitive for cells to pass through, while plasma can pass the filter matrix uninhibited. In some instances, the filter matrix comprises a membrane that is especially suited for this application because it combines a large pore size at the top with a small pore size at the bottom of the filter, which leads to very gentle treatment of the cells preventing cell degradation during the filtration process.

In some instances, the filter or membrane separates substances in the sample based on size, for example, the filter or membrane has a pore size that excludes a cell but is permeable to cell-free nucleic acids. Therefore, the plasma or serum can move more rapidly than a blood cell through the filter or membrane, and the plasma or serum containing any cell-free nucleic acids permeates the holes of the filter or membrane. In some instances, the cell that is slowed and/or trapped in the filter or membrane is a red blood cell, a white blood cell, or a platelet.

In some instances, the filter or membrane is capable of slowing and/or trapping a cell without damaging the cell, thereby avoiding the release of cell contents including cellular nucleic acids and other proteins or cell fragments that could interfere with subsequent evaluation of the cell-free nucleic acids. In some instances, at least 95%, at least 98%, at least 99%, or up to 100% of the cells in a blood sample remain intact when trapped in the filter or membrane. In addition to or independently of size separation, the filter or membrane can trap or separate unwanted substances based on a cell property other than size, for example, the filter or membrane can comprise a binding moiety that binds to a cell surface marker. In some instances, the binding moiety is an antibody or antigen binding antibody fragment. In some instances, the binding moiety is a ligand or receptor binding protein for a receptor on a blood cell or microvesicle.

Examples of filter or membrane materials used in the device to remove cells include, but are not limited to, polyvinylidene difluoride, polytetrafluoroethylene, acetylcellulose, nitrocellulose, polycarbonate, polyethylene terephthalate, polyethylene, polypropylene, glass fiber, borosilicate, vinyl chloride, silver. Suitable filter or membranes may be characterized as preventing passage of cells. In some instances, the filter or membrane is a hydrophobic filter, for example a glass fiber filter, a composite filter, for example Cytosep (e.g., Ahlstrom Filtration or Pall Specialty Materials, Port Washington, N.Y.), or a hydrophilic filter, for example cellulose (e.g., Pall Specialty Materials).

In some instances, the filter or membrane is characterized by at least one pore size. In some instances, the device comprises multiple filters and/or membranes, wherein the pore size of at least a first filter or membrane differs from a second filter or membrane. In some instances, at least one pore size of at least one filter/membrane is about 0.05 microns to about 10 microns. In some instances, the pore size is about 0.05 microns to about 8 microns. In some instances, the pore size is about 0.05 microns to about 6 microns. In some instances, the pore size is about 0.05 microns to about 4 microns. In some instances, the pore size is about 0.05 microns to about 2 microns. In some instances, the pore size is about 0.05 microns to about 1 micron. In some instances, at least one pore size of at least one filter/membrane is about 0.1 microns to about 10 microns. In some instances, the pore size is about 0.1 microns to about 8 microns. In some instances, the pore size is about 0.1 microns to about 6 microns. In some instances, the pore size is about 0.1 microns to about 4 microns. In some instances, the pore size is about 0.1 microns to about 2 microns. In some instances, the pore size is about 0.1 microns to about 1 micron.

In some instances, the laminate further comprises an adhesive layer 306. The adhesive layer 306 may control the active area of the membrane or filter layer 304. In some instances, the laminate comprises a membrane support. The membrane support 308 layer may comprise a channel for plasma to move away from the membrane or filter 304 and towards the transfer channel layer 310. In some cases, the membrane support layer 308 is configured to minimize the distance that plasma travels from an area on the membrane or filter layer to reach a channel in the transfer channel layer 310. The transfer channel layer 310 may provide fluidic communication between the membrane support layer 308 to the channel transfer layer 310. In some cases, the channel transfer layer may be in fluidic communication with a collection vessel 102. In some instances, the collection vessel may comprise a PCR tube.

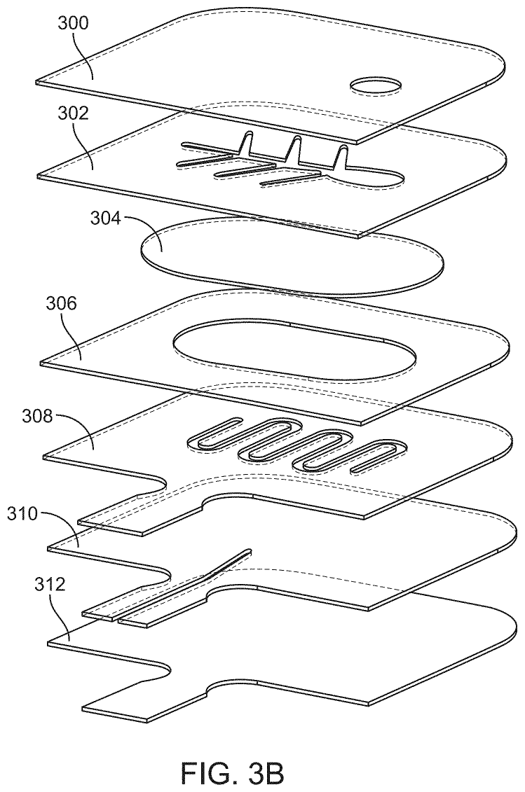

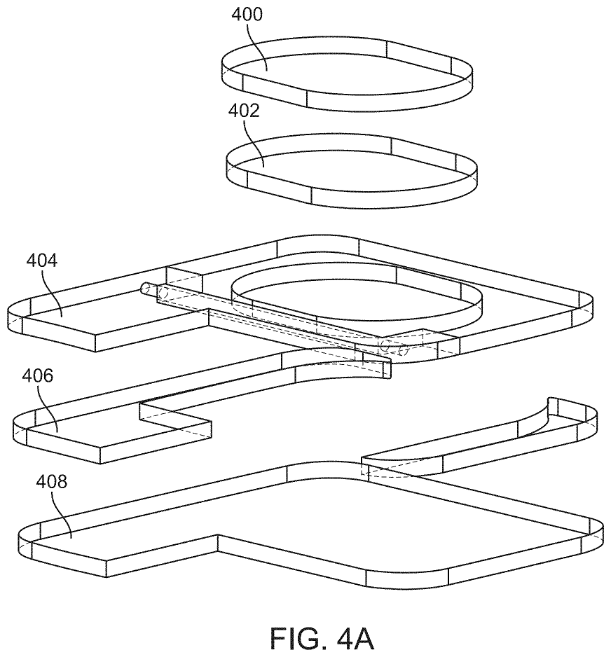

In some cases, the device may comprise a cartridge 104, as seen in FIGS. 4A-4D. In some instances, the cartridge may be configured to apply a positive pressure to the blood sample deposited into the laminate 106. Any type of positive pressure source may be used, and is not limited to the specific embodiments described herein. In some cases, the positive pressure source may comprise a mechanical positive pressure source. In some cases, the positive pressure is generated through the compression of a foam material. In such cases, the foam material may be compressed through mechanical displacement provided by the housing rails (504, 505), as seen in FIG. 5C against the cartridge force transducer 400, thus compressing the foam material 402 of the cartridge 104, seen in FIGS. 4A-4D. In some cases, the positive pressure source may comprise a material that possesses a spring force. In some instances, the positive pressure source may comprise a compression foam 402. In some instances, the cartridge 104 may comprise a plurality of structural layers (404, 406, 408) that provide structural rigidity of the cartridge 104. In some cases, the plurality of structural layers (404, 406, 408) may comprise a layer that houses the cartridge force transducer 400 and the compression foam 402, as seen in FIG. 4A. In some cases, a layer 404 of the plurality of structural layers (404, 406, 408) may comprise a hinge configured to open or seal (FIG. 4D) the blood sample deposited on the laminate 106.

In some cases, the device may comprise a housing 100, as seen in FIG. 5A. In some instances, the housing 100 may be configured to provide a positive pressure when in contact with the cartridge force transducer 400 and compression foam 402. In some cases, the housing 100, may comprise a top 502 and bottom portion 506, as seen in FIG. 5B and FIG. 5C, respectively. The top portion of the housing 502 may comprise a plurality of rails 504 configured to compress the cartridge force transducer 400 and the compression foam 402. In some cases, the bottom portion of the housing 506, may comprise a plurality of rails 508 configured to support and/or compress the plurality of structural layers of the cartridge (404, 406, 408). The top and bottom portions of the housing (502, 506) may be fastened or linked together with fasteners through the plurality of fastener holes 505.

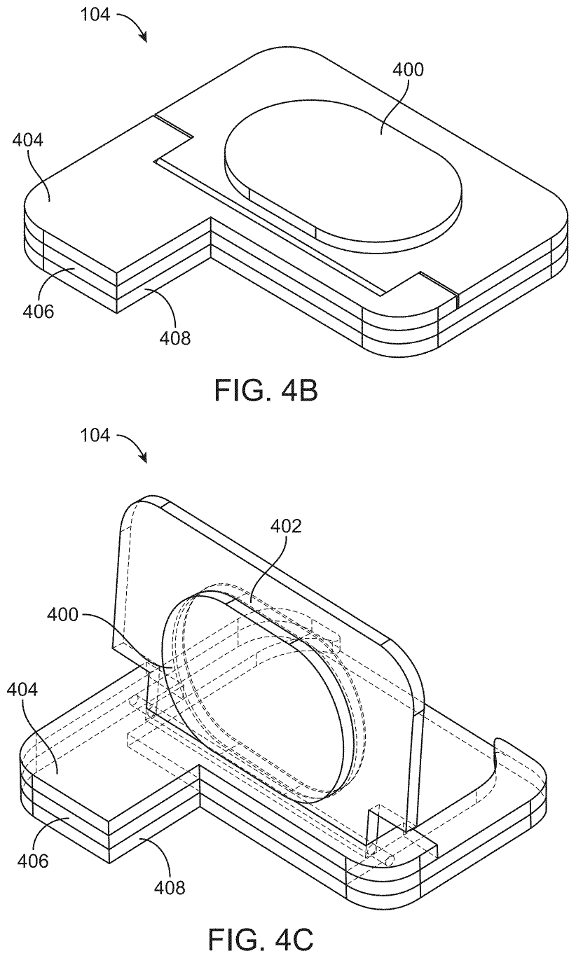

In some embodiments, the device may be configured to separate plasma from a blood sample (e.g., obtained from a finger prick). As can be seen in FIGS. 6A-6G, the laminate 106, cartridge 104, and collection vessel 102, may be assembled in preparation to receive a blood sample. In some cases, the laminate 106, cartridge 104, and collection vessel 102, may be inserted into the housing 100 to prevent spilling of the blood sample deposited onto the laminate 106. A blood sample may be deposited (e.g., into the sample inlet 300) and the cartridge 104 may be sealed over the laminate 106 in preparation to insert the cartridge 104, laminate 106, and collection vessel 102 assembly into the housing 100 configured to exert a positive pressure on the blood sample. Alternatively, the sample inlet 300, may be configured to be open during exertion of the positive pressure on the blood sample. The cartridge 104, laminate 106, and collection vessel 102 may be inserted into the housing 100, as seen in FIG. 6F. The insertion of the cartridge 104, laminate 106, and collection vessel 102 may induce a positive pressure from the top plurality of rails 504 to the cartridge force transducer 400 that then applies pressure to the compression foam 402 eventually applying positive pressure to the sample deposited on the laminate 106. The positive pressure on the blood sample pushes the blood sample into and through the filter or membrane such that plasma is separated from the blood sample (and other blood components are trapped or blocked from passing through) and passed towards the collection vessel 102. In some cases, once the cartridge 104, laminate 106, and collection vessel 102 have been inserted into the housing 100, the collection vessel 102, now comprising blood plasma, may be removed from the fluidic coupling of the laminate 106. In some cases, the blood plasma may then be utilized for further biological assays or methods described elsewhere herein.

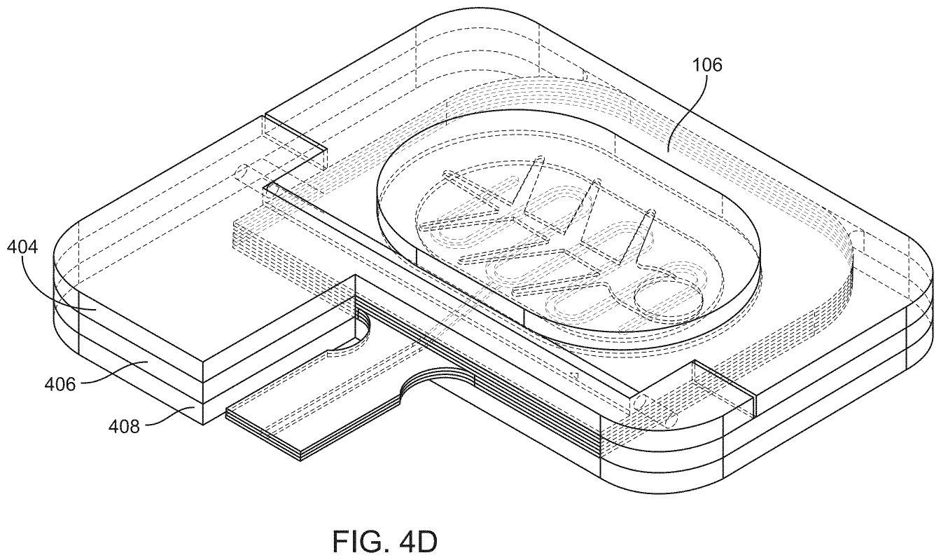

In some embodiments, the disclosure provided herein may comprise a laminate 700 configured to receive larger amounts of blood that may be collected in a traditional blood collection vessel 702, as seen in FIG. 7A. In some instances, the laminate configured to receive larger amounts of blood may be utilized with a larger cartridge 704, seen in FIG. 7B-7C.

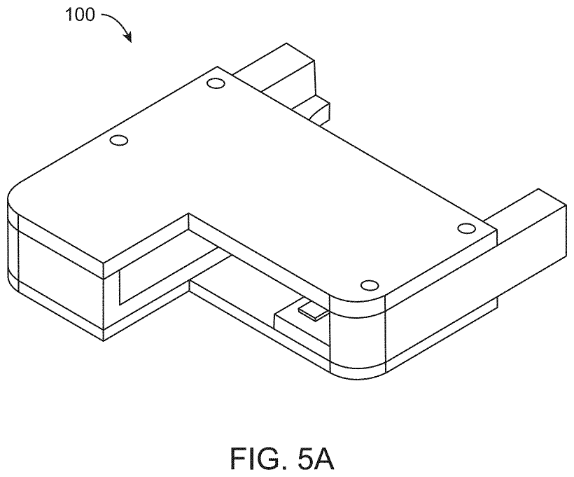

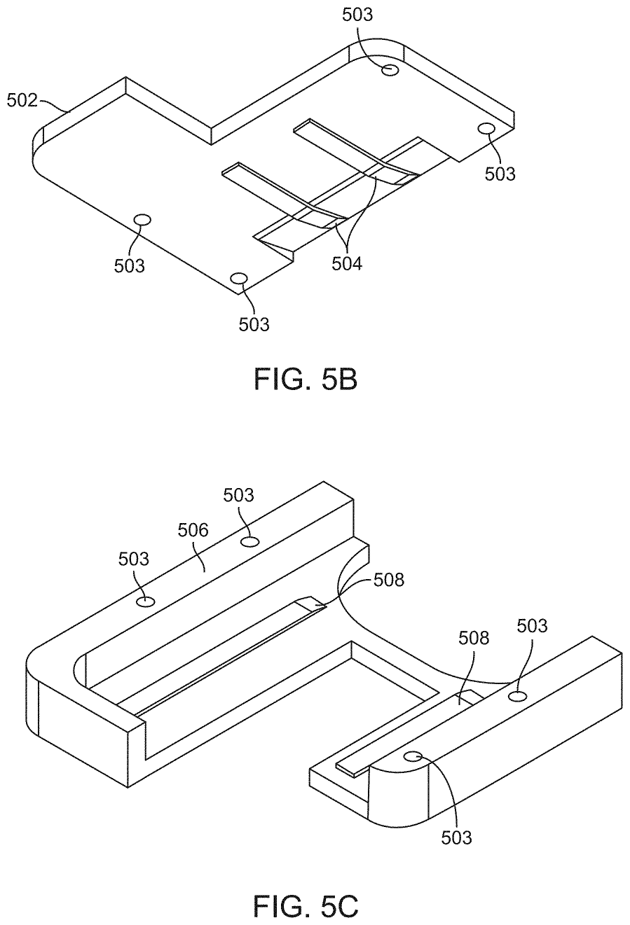

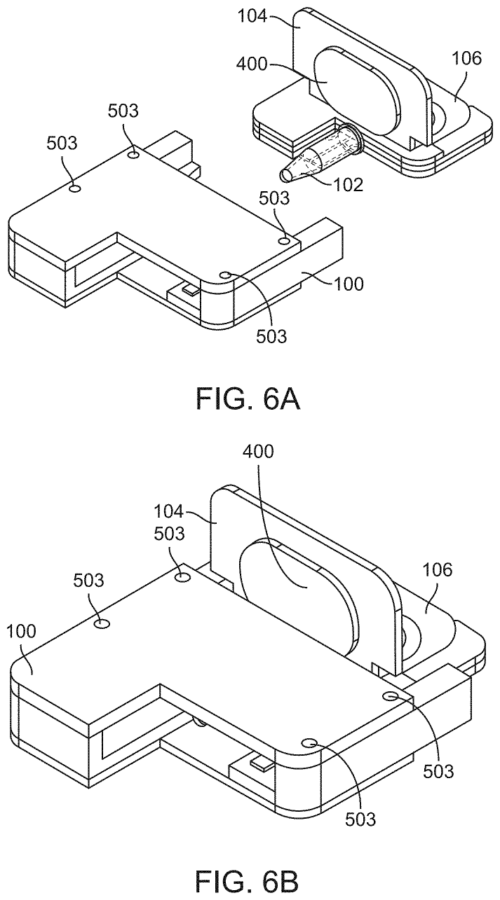

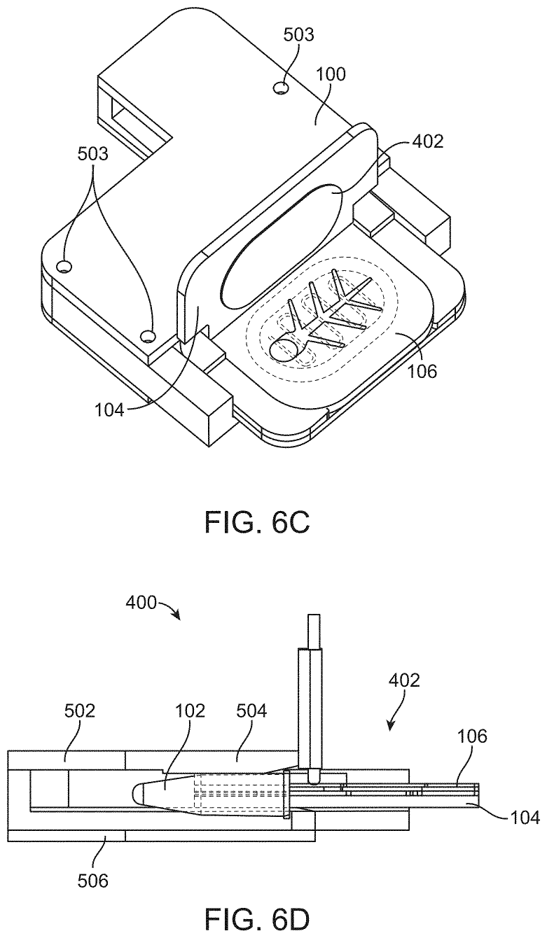

In some embodiments, the disclosure provided herein describes a plasma separation device 1030 configured to use positive pressure as described herein (e.g. as depicted in FIG. 8-FIG. 30). In some embodiments, the plasma separation device 1030 comprises a shipping sleeve 1031, a slider 1032, a cartridge base 1033, a laminate 1034, and a vial 1035. In some embodiments, the laminate 1034 comprises a blood inlet layer 1036, a blood metering layer 1037, a separation membrane layer 1038, a membrane support layer 1039, a transfer channel layer 1040 and a base layer, as depicted in FIG. 11. In some embodiments, the blood inlet layer comprises a 0.005 inch thick layer of polycarbonate. In some embodiments, the blood metering layer comprises a 0.0005 inch thick adhesive and a 0.002 inch thick polyester. In some embodiments the membrane support layer comprises a 0.002 inch thick polyester and a 0.0005 inch thick adhesive. In some embodiments, the transfer channel layer comprises a 0.002 inch thick polyester. In some embodiments, the base layer comprises a 0.002 inch thick polyester and a 0.0005 inch thick adhesive.

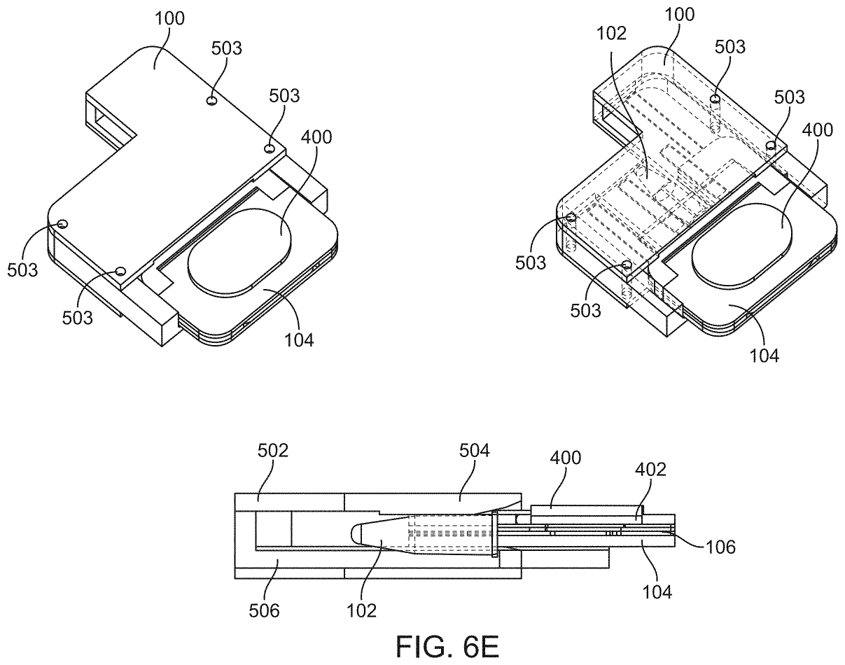

In some embodiments, the blood inlet layer 1036 comprises a blood inlet hole 1041. In some embodiments, the top inlet serves as an inlet where blood can be deposited. In some embodiments, the blood inlet hole 1041 is small to protect the remaining surface area, e.g. from contamination. In some embodiments, the blood inlet hole 1037 comprises arms 1042 that radiate outward to aid in the blood entering the stack (e.g. as depicted in FIG. 12). In some embodiments, the laminate does not comprise a blood inlet layer 1036.

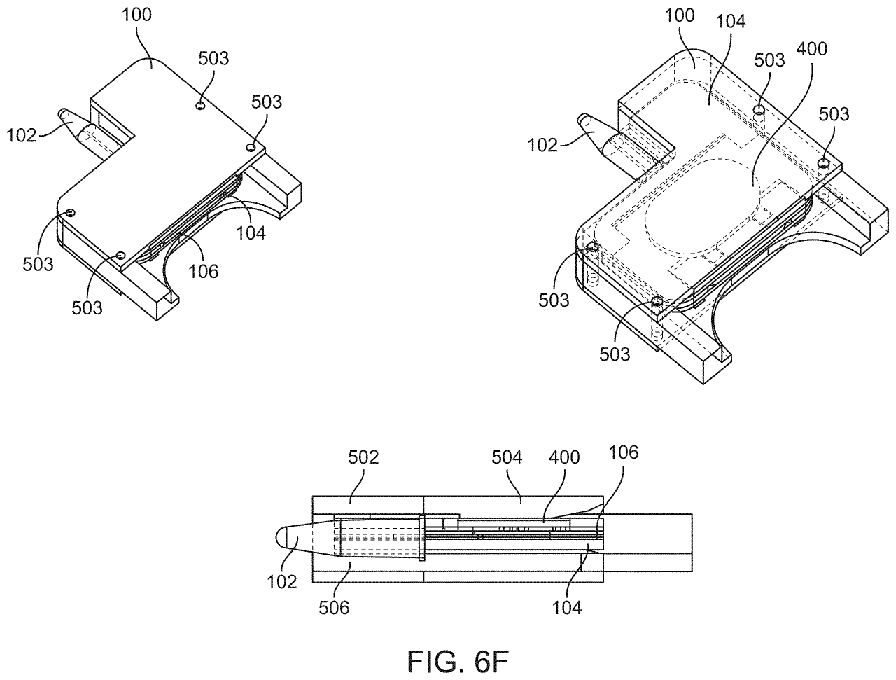

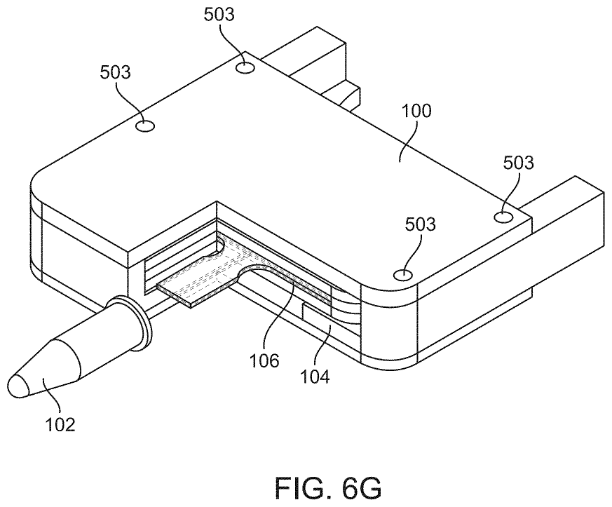

In some embodiments, the blood metering layer 1037 is a structure to meter the blood, as depicted in FIG. 13. In some embodiments, the blood metering layer contains excess blood that is not processed by the separation membrane. In some embodiments, the blood metering layer contains any blood over 100 μL. In some embodiments, a preservative 1045 is deposited in the blood metering layer 1037 as depicted in FIG. 14. In some embodiments, the preservative comprises EDTA.

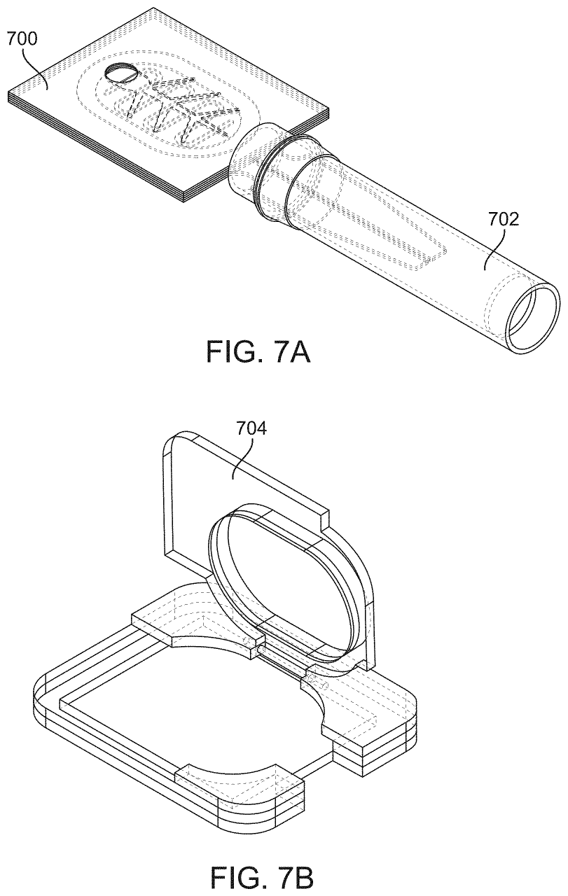

In some embodiments, the separation membrane layer 1038 comprises a series of pores, as depicted in FIG. 15. In some embodiments, the separation membrane layer comprises a membrane as described herein. In some embodiments, pores on the surface facing the top layer are larger and pores facing the membrane support layer are smaller. In some embodiments, the smallest pores are too small for cells to pass through. In some embodiments, the capacity of the separation membrane is 50 μL of blood per square centimeter of membrane.

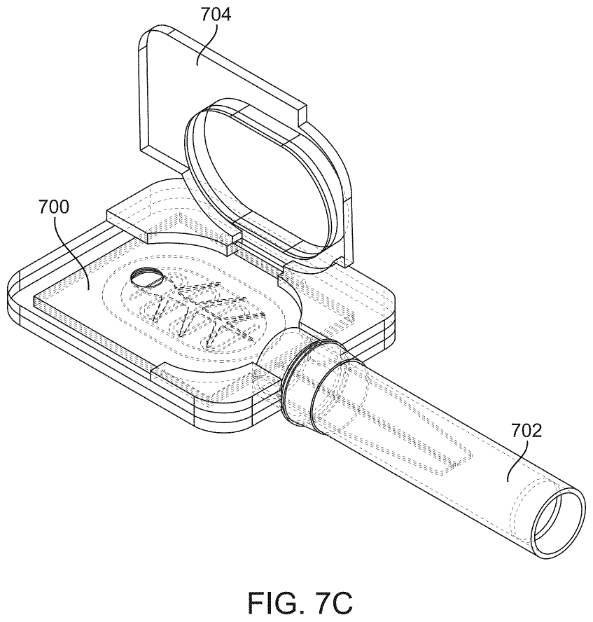

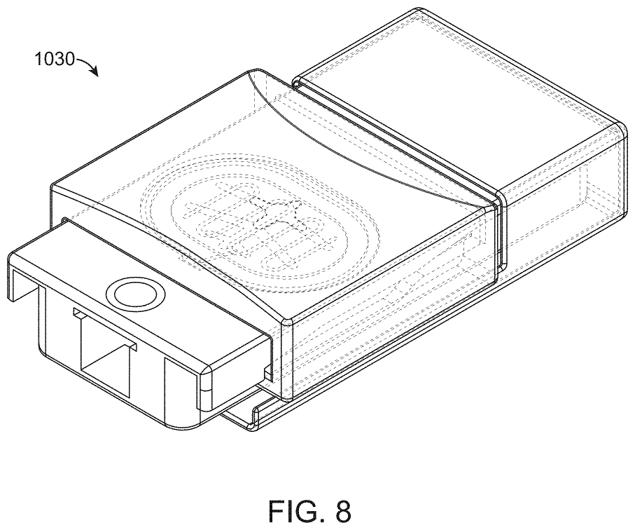

In some embodiments, the membrane support layer 1039 comprises a channel 1043 for the plasma to move away from the separation membrane as depicted in FIG. 16. In some embodiments, plasma is processed directly into the channel and can be moved out. In some embodiments, plasma is pushed into the channel by additional channel. In some embodiments, the distance that plasma needs to travel from any areas on the membrane to the channel 1043 is minimized. In some embodiments, a preservative 1044 is deposited in the channel 1043, such as in FIG. 17. In some embodiments, the preservative is EDTA.

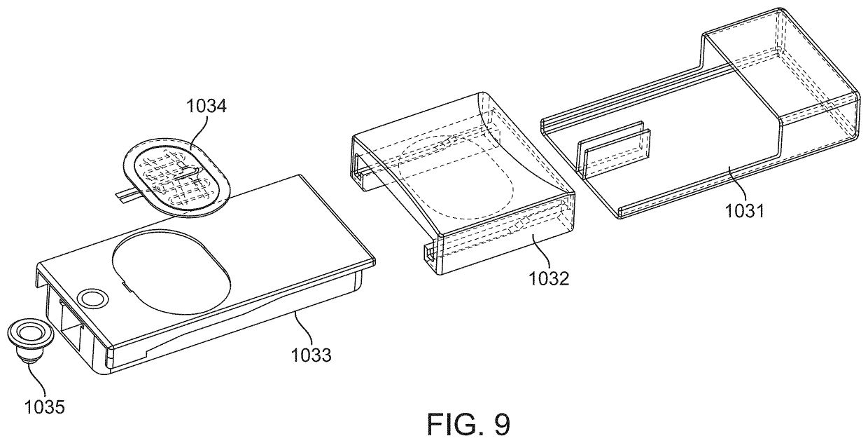

In some embodiments, the plasma from the channel 1043 of the membrane support layer 1039 moves into the transfer channel layer 1040 as depicted in FIG. 18. In some embodiments, the transfer channel layer comprises a transfer channel 1046 which moves the plasma from the membrane support layer 1039 to a collection vial. In some embodiments, the transfer channel 1046 moves the plasma into a larger tube or a length of tubing.

In some embodiments, the base layer is a rigid or semi-rigid piece for construction.

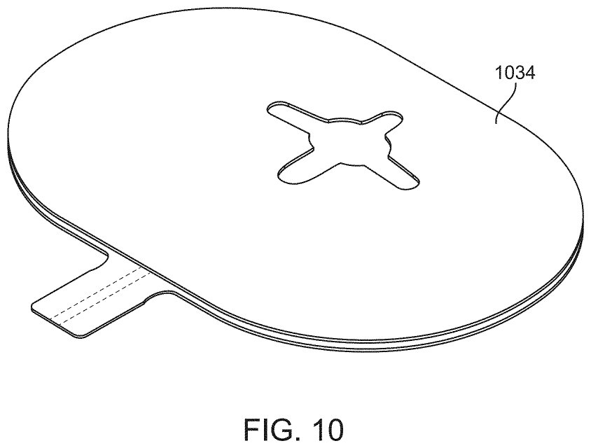

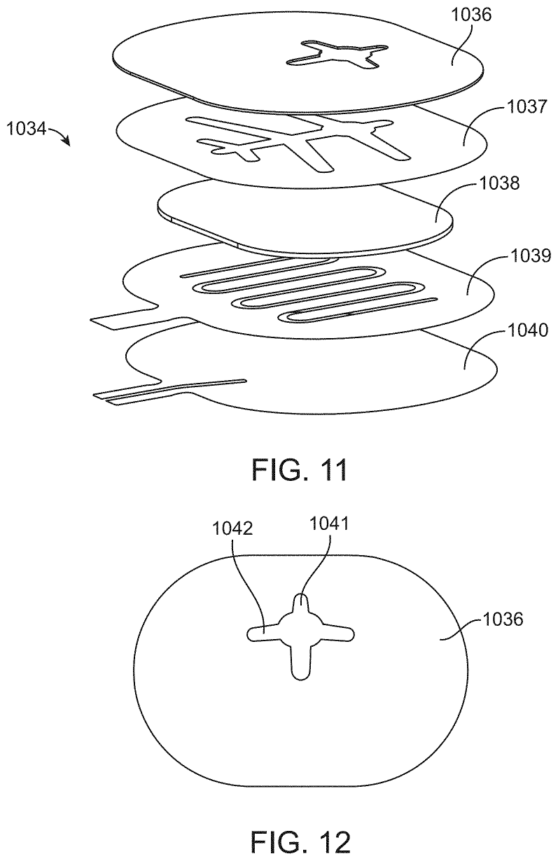

In some embodiments, the cartridge base 1033 is the main structural component of the plasma separation device. In some embodiments, the base 1033 supports the laminate 1034 as depicted in FIG. 19. In some embodiments, the base holds the laminate in the appropriate position to deliver plasma into the collection vial. In some embodiments, the cartridge base acts with the slide to apply pressure to the blood that is to be processed. In some embodiments, the cartridge base 1033 holds the laminate 1034 and the vial 1035 as depicted in FIG. 20. In some embodiments, a preservative is coated in the laminate. In some embodiments a preservative is in the vial.

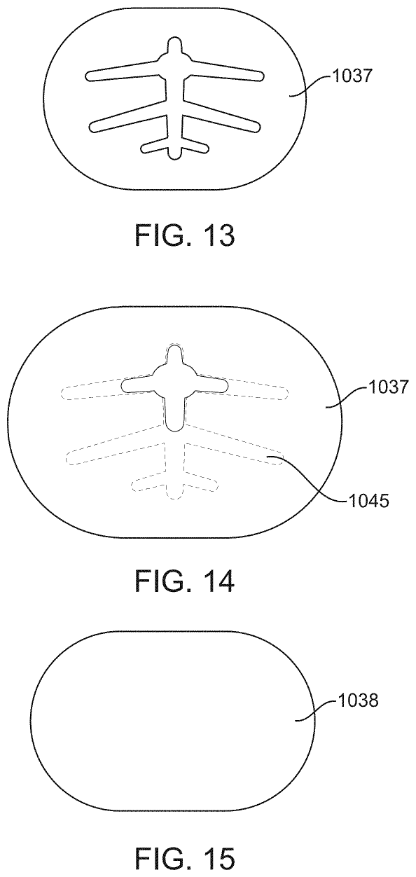

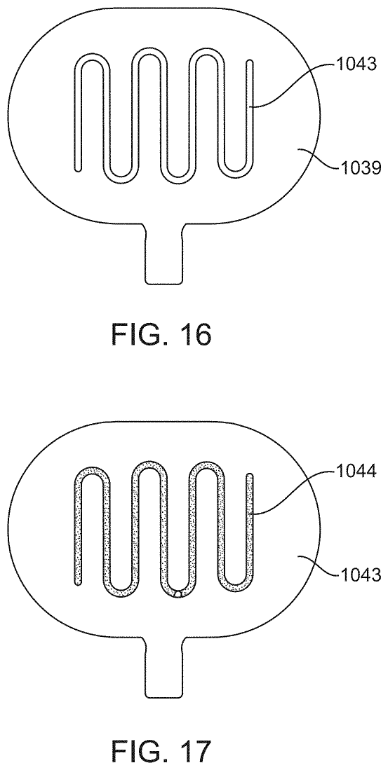

In some embodiments, the vial 1035 is sealed to the base 1033. In some embodiments, the vial 1035 is sealed to the base 1033 by way of an O-ring or a molded gasket. In some embodiments, there are two positions for the vial 1035 in the cartridge base 1033, as depicted in FIG. 21. In some embodiments, the collection position comprises the vial 1035 in a first position 1047 in the cartridge base 1033 so that the laminate can deliver plasma to the vial. In some embodiments, the storage position comprises the vial 1035 in a second position 1048 in the cartridge base 1033 so that the vial is sealed against the cartridge base. In some embodiments, a foil seal 1049 in the cartridge base 1033 allows access to the collection vial, as depicted in FIG. 22. In some embodiments, the cartridge base 1033 comprises a rail 1050.

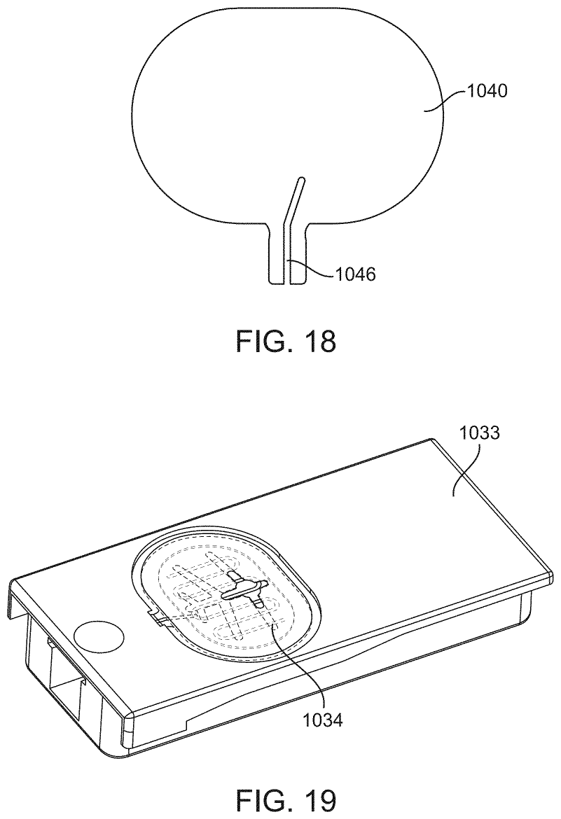

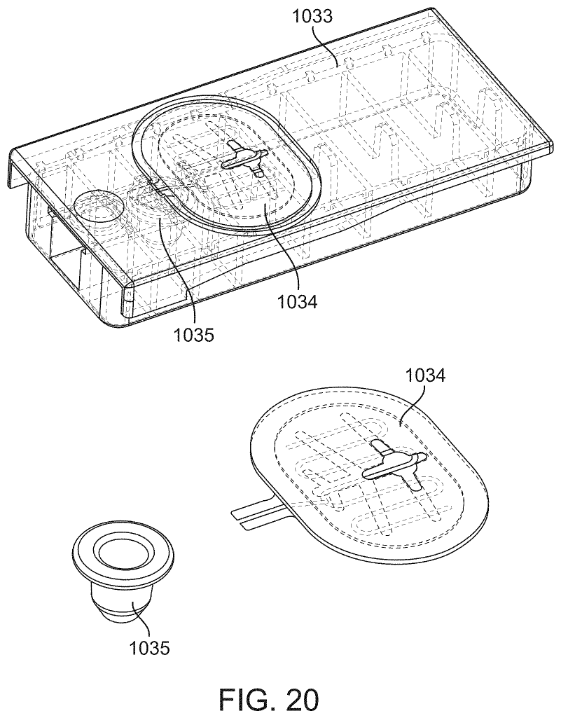

In some embodiments the slider 1032, such as depicted in FIG. 23 and FIG. 24, engages with the cartridge base 1033. In some embodiments, the slide 1032 engages with the cartridge base 1033 to apply pressure to the whole blood in order to separate the plasma. In some embodiments, the slider comprises a compression material 1051. In some embodiments, the compression rails on the slider 1052 engage with the rail 1050 on the cartridge base 1033 to generate pressure at the compression material on the laminate stack. In some embodiments, the compression material comprises a compression foam as described herein. In some embodiments, the compression material comprises any material that possess a spring force. In some embodiments, the compression foam applies the force to the laminate. In some embodiments, the spring force of the compression foam is configured to translate the displacement of the compression insert to a pressure. In some embodiments, the compression foam starts an initial thickness and when the compression foam is compressed, it transfers a pressure based on its density. In some embodiments, the compression foam is 1.57 mm. In some embodiments, when the compression foam is compressed to 0.27 mm (12% of its initial thickness) it applies a pressure of 12 psi. In some embodiments, the compression foam generates a pressure less than 4 psi.

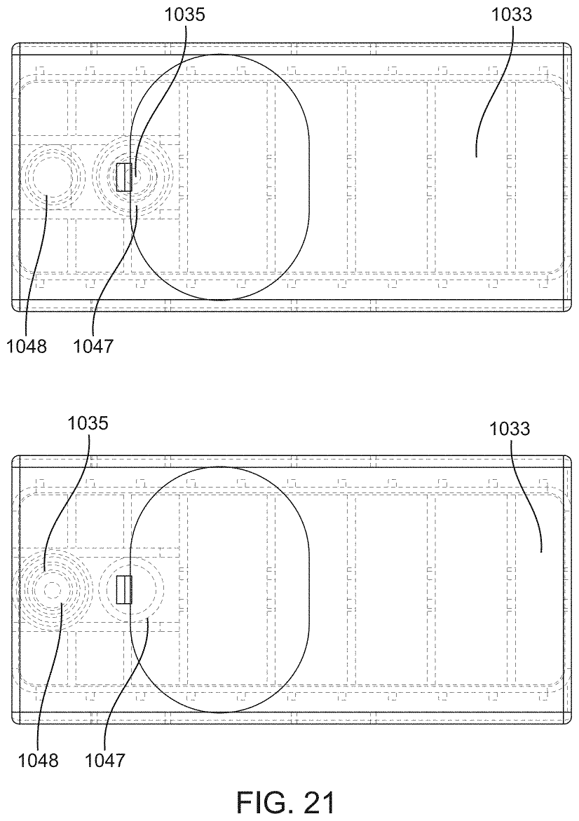

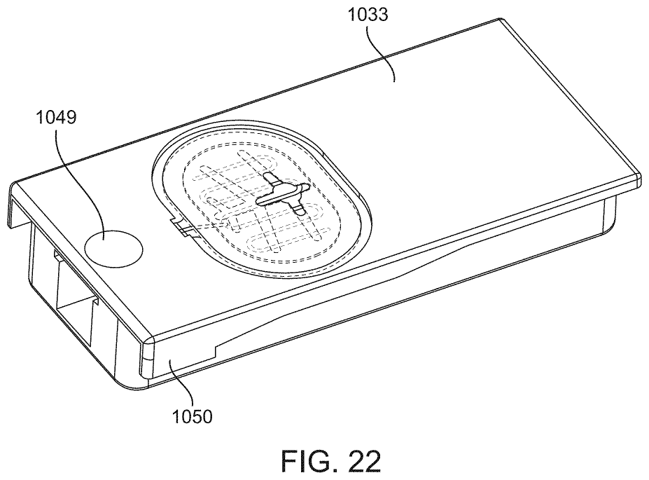

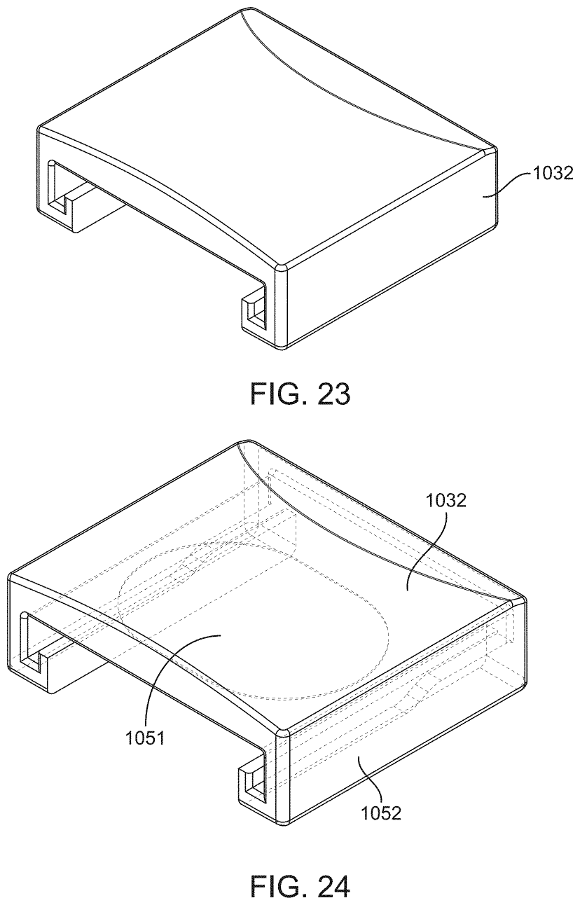

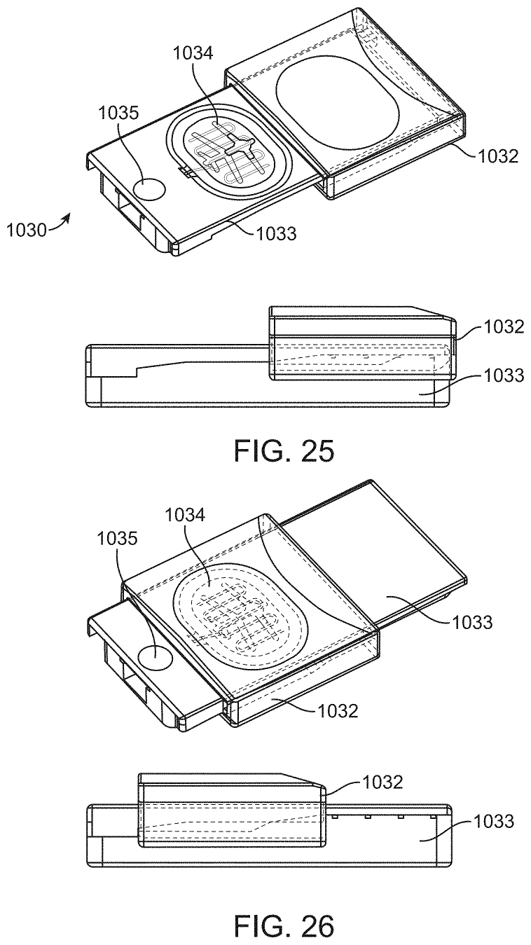

In some embodiments, the slider 1032 is configured to reveal the laminate 1034 for application of whole blood, as depicted in FIG. 25. In some embodiments, the slider 1032 is configured to cover the laminate 1034 for shipping, as depicted in FIG. 26. In some embodiments, when the slider 1032 is moved from the position for blood application 1053 to the position for plasma separation 1054, the interaction of the rails on the base 1050 and the compression rails on the slider 1052 cause the slider to move down, as depicted in FIG. 27. In some embodiments, this downward movement results in pressure applied to the whole blood contained in the laminate 1034. In some embodiments, when the slider is moved from the position for blood application 1053 to the position for plasma separation 1054, the vial 1035 remains stationary as depicted in FIGS. 28A and 28B.

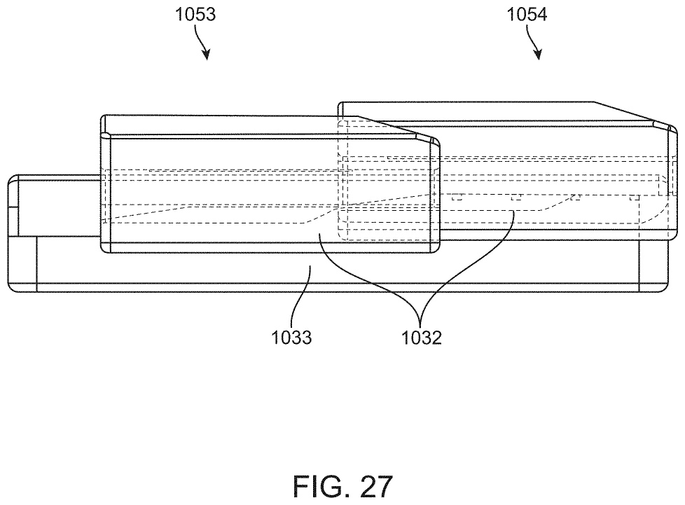

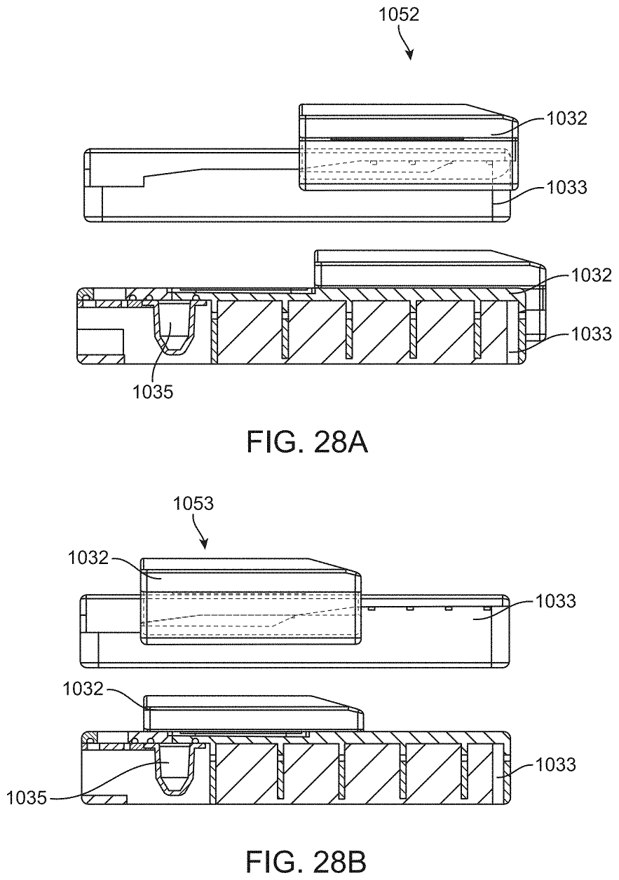

In some embodiments, the device comprises a shipping sleeve 1031. In some embodiments, once the slider is in the position for plasma separation 1054, the shipping sleeve 1031 can be installed onto the cartridge base 1033, as depicted in FIG. 29. In some embodiments, when the shipping sleeve 1031 is installed onto the cartridge, the vial is moved from the collection position 1055 to the storage position 1056 as depicted in FIG. 30.

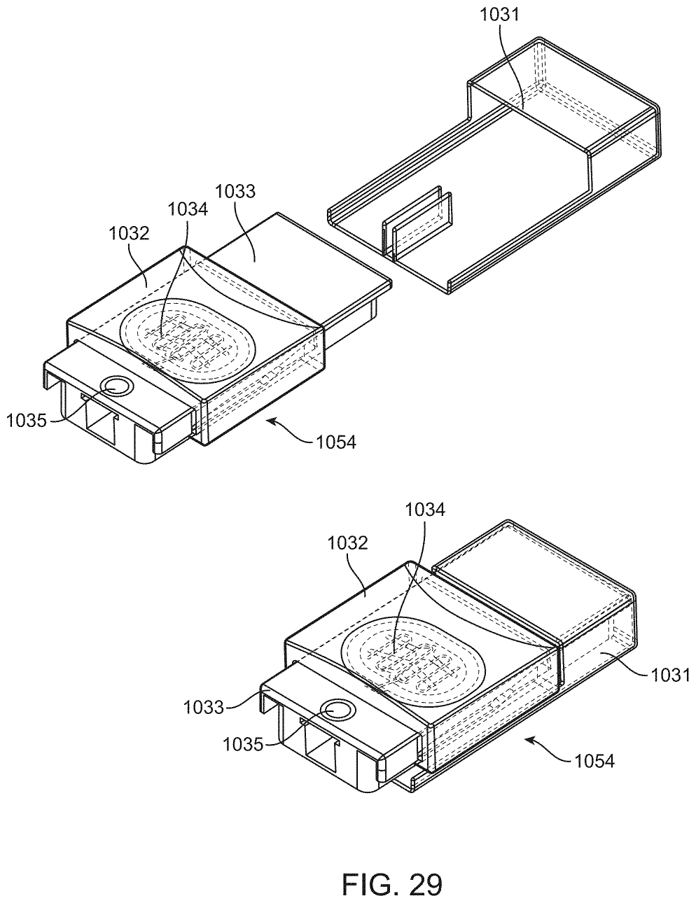

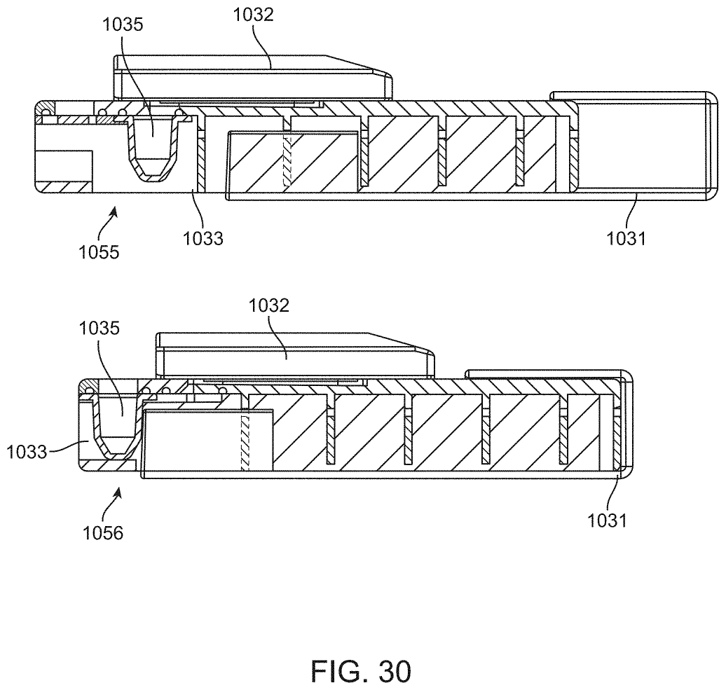

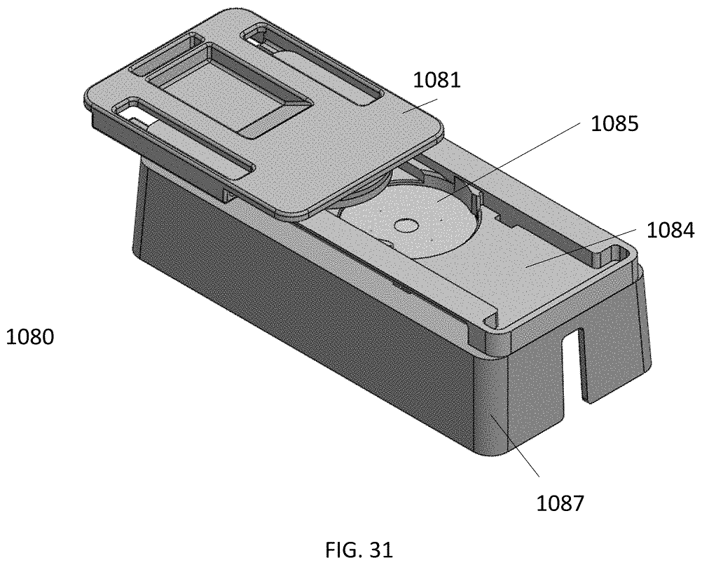

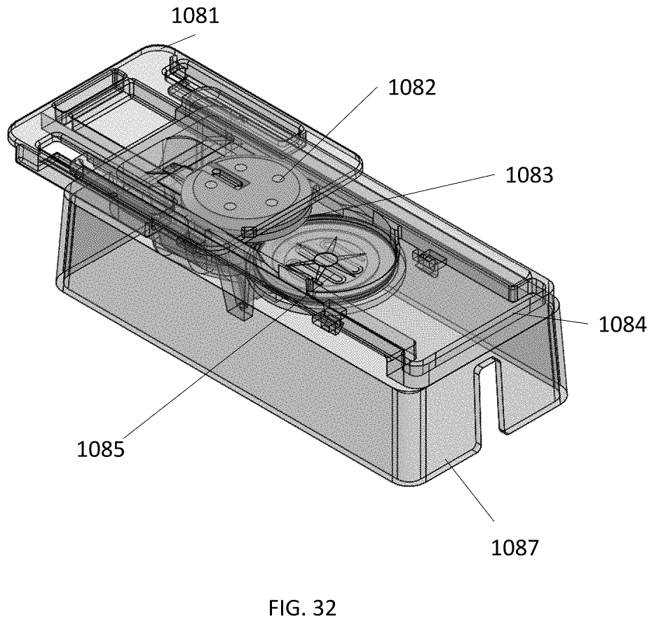

In another embodiment, the device 1080 is depicted in FIG. 31-FIG. 33. In some embodiments, the device comprises a slider 1081, a crush disc 1082, a crush elastomer 1083, an enclosure lid 1084, a laminate layer 1085, a plasma collection tube 1086 and an enclosure base 1087. In some embodiments, the device further comprises a shipping container as depicted in FIG. 34. In some embodiments, the shipping container comprises a shipping container lid 1088 and a shipping container base 1089. In some embodiments, the device allows plasma to be separated from whole blood.

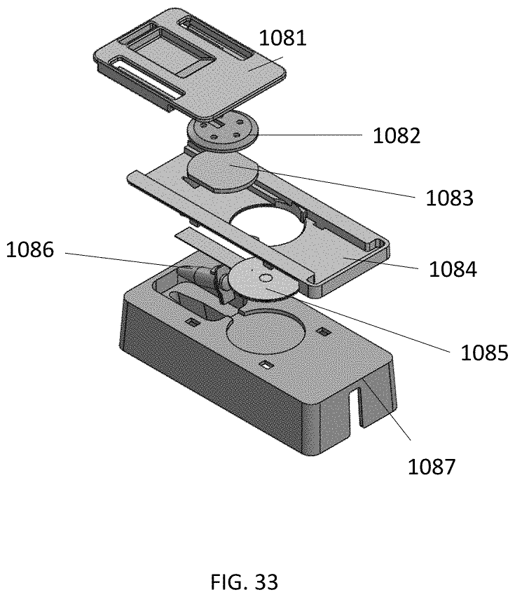

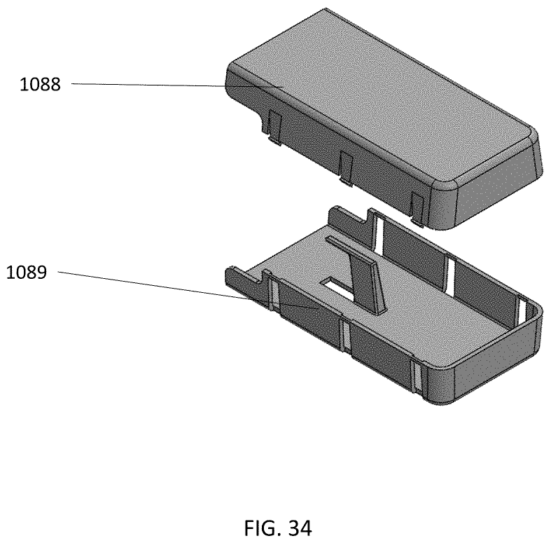

In some embodiments, the device comprises a laminate as described herein. In some embodiments, the device comprises a laminate 1085 as depicted in FIGS. 35 and 36. In some embodiments, the laminate 1085 comprises a blood inlet layer 1090, a blood metering layer 1092, a separation membrane 1093, an adhesive layer 1094, a membrane support layer 1095, a transfer channel layer 1096, and a base layer 1097.

In some embodiments, the blood inlet layer comprises a polycarbonate film (e.g., 5 mm). In some embodiments, the blood inlet layer 1090 comprises a blood inlet 1098 and arms 1099 extending outward. In some embodiments, the blood droplet enters the laminate stack 1085 through the blood inlet 1098 and air is able to escape through the arms 1099.

In some embodiments, the blood metering layer comprises a single-side adhesive (e.g., 2.5 mil) with the adhesive facing towards the blood inlet layer. In some embodiments, the membrane comprises a certain capacity for blood. When blood is deposited in an excessive amount, the membrane may take up the certain capacity of blood, some amount of blood may rest in this metering feature, and the remaining amount may sit outside of the inlet port and may not be processed through the membrane. In some embodiments, the maximum amount of blood that enters the blood inlet port is approximately 150 μl and any amount above this is pushed away and does not get processed.

In some embodiments, the separation membrane 1093 comprises Pall Vivid plasma separation membrane GR grade material. In some embodiments, the membrane comprises a series of pores with larger pores on the surface closer to the blood inlet and pore size reducing further down the membrane. In some embodiments, the pores on the side of the membrane layer facing the adhesive layer are too small for cells to pass through. In some embodiments, the capacity of the membrane is 50 μL of blood for 1 cm² of membrane.

In some embodiments, the adhesive layer 1094 comprises Adhesive Research ARSeal 90880 5.6 mil double sided silicone adhesive. In some embodiments, the adhesive layer controls the active area of the separation membrane layer 1093. In some embodiments, the separation membrane layer 1093 is oversized, but the perimeter of the separation membrane layer is needed to seal the inlet side from the output side. The active area of the membrane that is available to process blood is controlled by the size of the opening of the adhesive layer 1094.

In some embodiments, the membrane support layer 1095 comprises a single sided adhesive (e.g., 2.5 mil) with the adhesive facing away from the blood inlet layer. In some embodiments, the membrane support layer 1095 comprises a channel 1098 for plasma to move away from the membrane. In some embodiments, plasma is processed directly onto the channel and is pushed through the channel by additional plasma. In some embodiments, the distance that plasma from any area on the membrane needs to travel to reach a channel is minimized. In some embodiments, the membrane support layer comprises a rectangular portion 1099 extending from the layer. In some embodiments, the rectangular portion serves as a top of the transfer channel.

In some embodiments, the transfer channel layer 1096 comprises a single sided adhesive (e.g., 2.5 mil) with the adhesive facing away from the blood inlet layer 1090. In some embodiments, the plasma from the channel 1098 in the membrane support layer 1095 then moves to the transfer channel 1100 and out to a collection tube 1086.

In some embodiments, the base layer 1097 comprises 5 mm polycarbonate film.

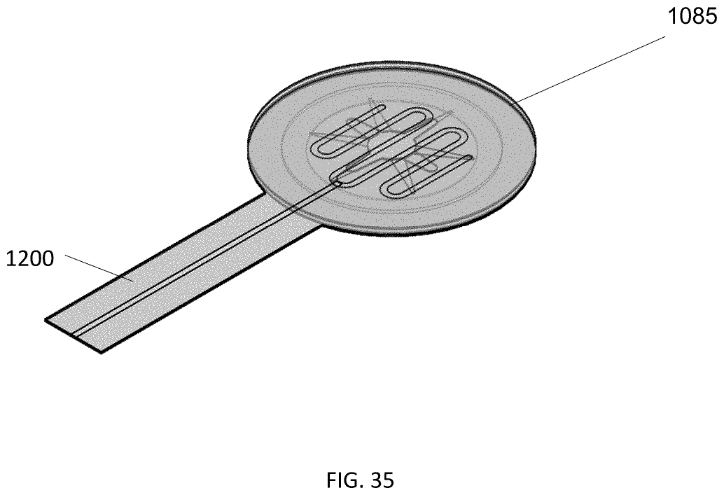

In some embodiments, as depicted in FIG. 37, the enclosure base 1087 holds the collection tube 1086 and laminate layers 1085. In some embodiments, the enclosure base comprises a collection tube cutout 1101. In some embodiments, the geometry of the collection tube cutout 1101 holds the lid 1400 of the tube 1086 in such a way as to scrape excess plasma off the laminate and into the collection tube when placed in the shipping container. In some embodiments, the enclosure base 1087 comprises a laminate nest 1102 configured to hold the laminate layer 1085. In some embodiments, the depth of the laminate nest 1102 is dictated by the required force to express plasma. In some embodiments, rectangular holes in the top surface 1103 allow the enclosure lid 1084 to be attached via snap features. In some embodiments, a rectangular cutout 1104 on one end allows the shipping container 1089 to close the lid of the collection tube 1086.

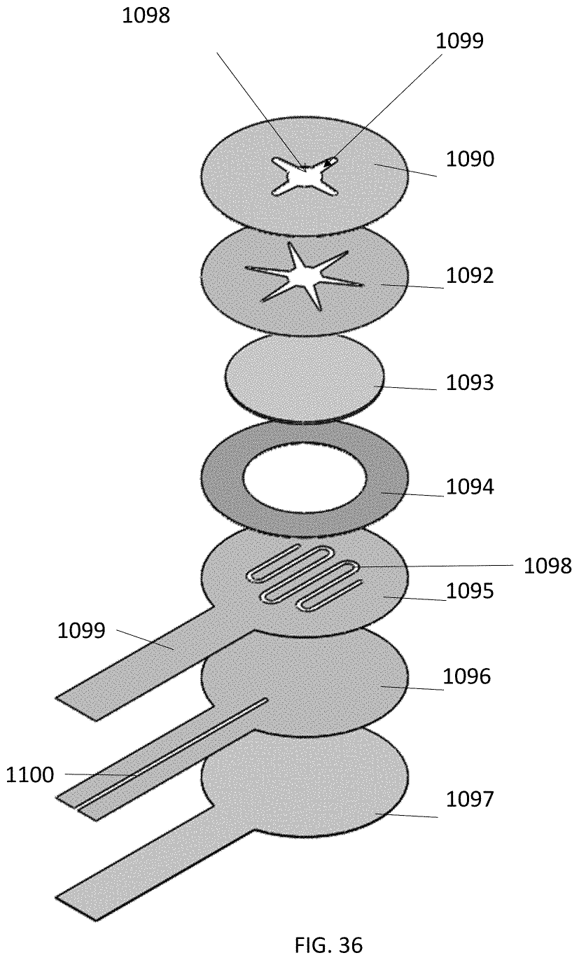

In some embodiments, as depicted in FIG. 38, the collection tube 1086 comprises a small tube with a closable lid 1400. In some embodiments, the outlet channel 1200 of the laminate is placed inside of the collection tube. In some embodiments, plasma is dispensed into the tube during actuation. In some embodiments, when the device is placed in the shipping container, the outlet channel is pushed out of the collection tube and excess plasma is scraped off the laminate by the closeable lid 1400 and deposited into the collection tube 1086. In some embodiments, once closed, the collection tube 1086 is leak tight and can be shipped via ground or air.

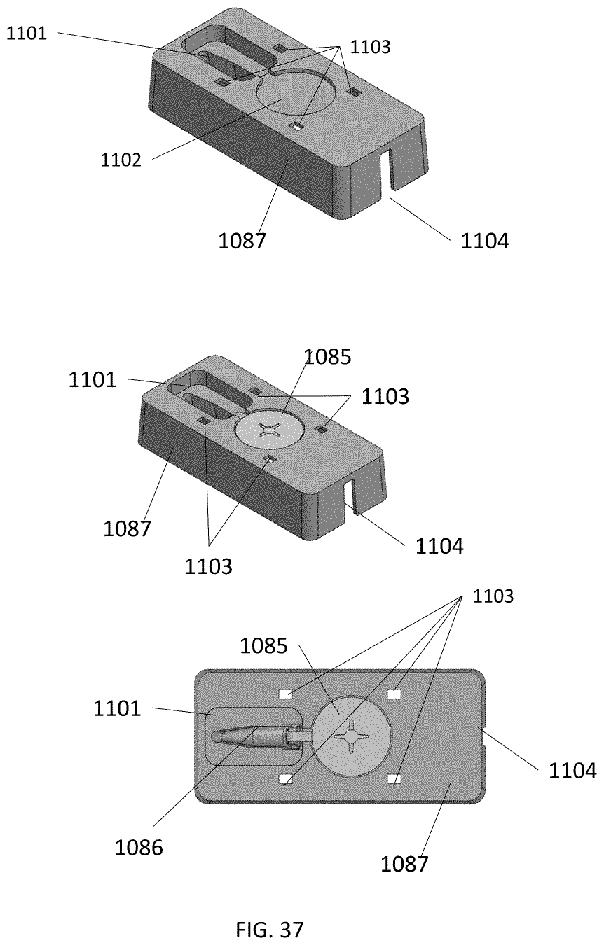

In some embodiments, the device described herein comprises an enclosure lid 1084 as depicted in FIG. 39. In some embodiments, the enclosure lid comprises outer rails 1201 and inner rails 1202. In some embodiments, the enclosure lid comprises a laminate cutout 1203. In some embodiments, the enclosure lid 1084 is configured to allow the slider 1081 and crush disc 1082 to be slid over and apply force to the laminate layer 1085. In some embodiments, the diameter of the laminate cutout 1203 is slightly smaller than the laminate layer 1085, constraining the laminate in place. In some embodiments, the enclosure lid comprises a collection tube brace 1206 configured to hold the collection tube in the proper position in the enclosure base.

In some embodiments, the outer rails 1201 guide the slider 1081. In some embodiments, height of these rails is dictated by the force required to express plasma. In some embodiments, small bumpers 1204 in the outer rails are configured to interface with grooves 1301 in the slider to hold the device in the open position to prevent accidental actuation. In some embodiments, the inner rails 1202 are configured to guide the crush disc/elastomer and prevent the elastomer from contacting anything but the laminate. In some embodiments, the enclosure lid comprises 4 snap features 1205 configured to allow this piece to be attached to the enclosure base 1087 via the rectangular holes 1103.

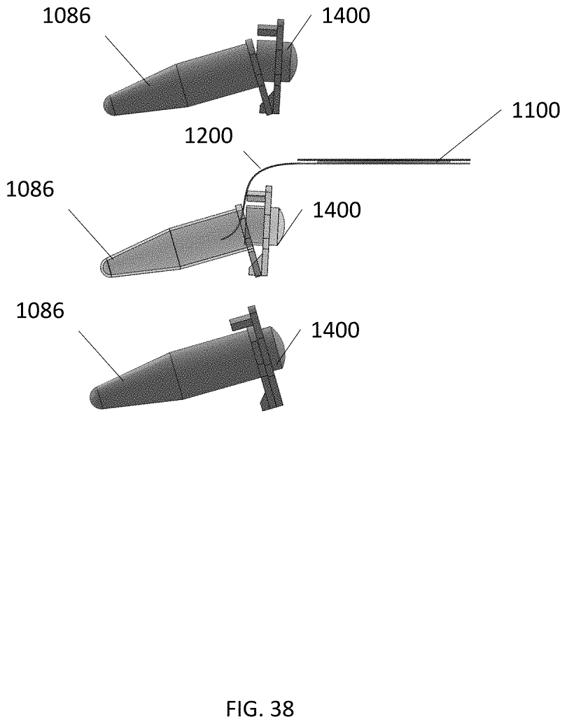

In some embodiments, the device described herein comprises a crush disc 1082, as depicted in FIG. 40. In some embodiments, the device comprise an elastomer 1083. In some embodiments, the crush disc and the elastomer are configured to distribute force to the laminate 1085 in order to express the plasma, In some embodiments, the crush disc comprise a rigid low friction plastic. In some embodiments, the crush disc comprises Delrin. In some embodiments, the crush disc is configured to allow force to be transferred from the slider 1081 to the laminate 1085 while allowing for an easy, non-binding sliding interface with the inner rails of the enclosure lid. In some embodiments, the crush disc 1082 comprises a notch 1207 configured to allow for proper alignment with the slider 1081. In some embodiments, the notch 1207 is configured to aid in sealing the laminate 1085 during actuation. In some embodiments, the crush disc 1082 comprises holes 1208. In some embodiments, the holes are configured to allow the elastomer 1083 to be overmolded onto the crush disc 1082. In some embodiments, the elastomer 1083 is configured to evenly distribute the force from the crush disc 1082 to the laminate layer 1085. In some embodiments, the elastomer comprises a soft durometer configured to ensure that no pressure hotspots occur on the laminate layer. In some embodiments, the elastomer comprises a soft durometer configures to act as a sealing surface to prevent blood leakage from the laminate. In some embodiments, the durometer is about 25 Shore A. In some embodiments, the elastomer 1083 comprises side cutouts 1209 configures to allow the crush disc 1082 to slide on the inner rails 1202 of the enclosure lid 1084 smoothly. In some embodiments, the elastomer 1083 is secured to the crush disc 1082 by means of over-molding or adhesive bonding.

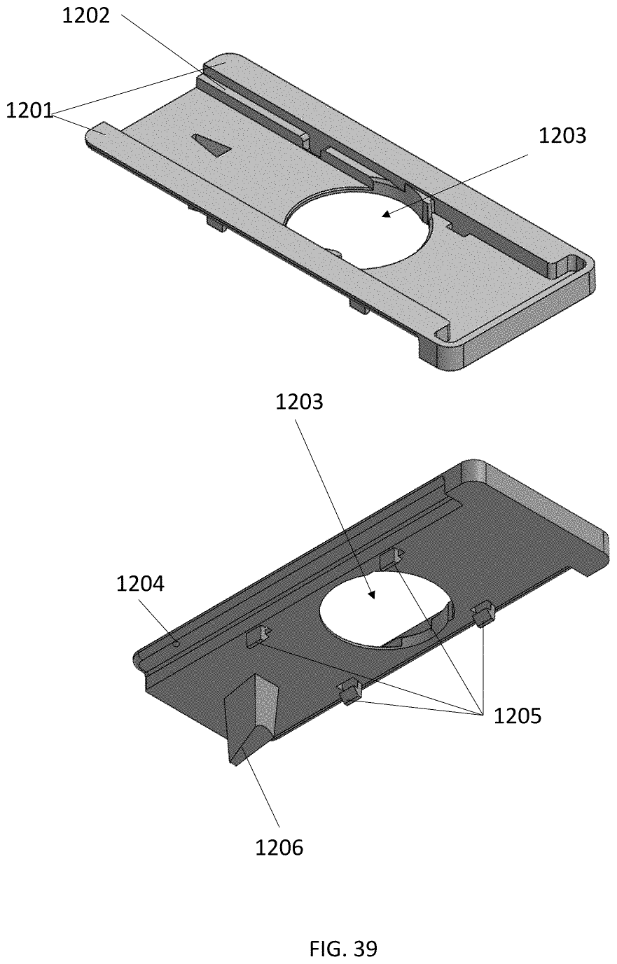

In some embodiments, the enclosure lid 1084 and crush disc 1082 are configured as depicted in FIG. 41. In some embodiments, in the open configuration, the crush disc 1082 is configured to rest on the inner rails 1202 of the enclosure lid 1084. In some embodiments, the crush disc 1082 is configured to hold the elastomer 1083 off of the enclosure lid 1084 to prevent binding during actuation. In some embodiments, when the device is active, the crush disc 1082 slides along the rails 1201 until it can drop into the laminate cutout 1203. In some embodiments, the crush disc 1082 is configured to apply force to the laminate 1085 when the crush disc is within the laminate cutout 1203.

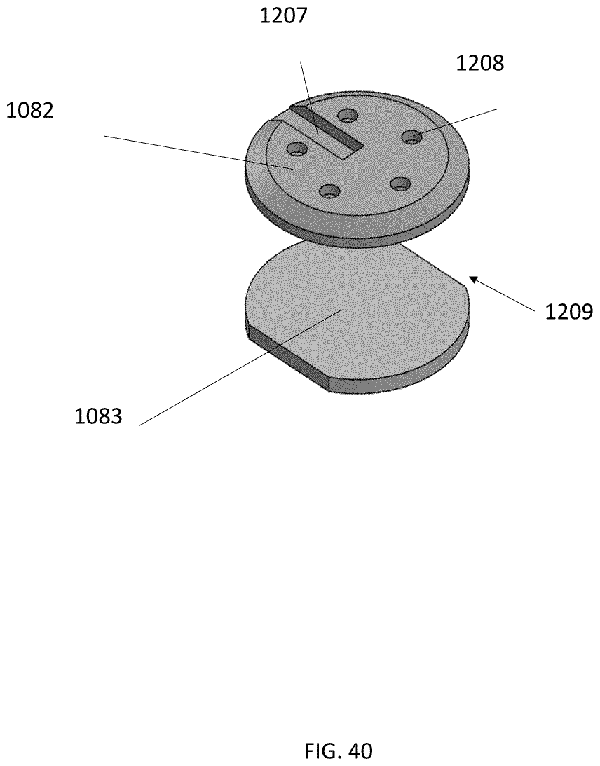

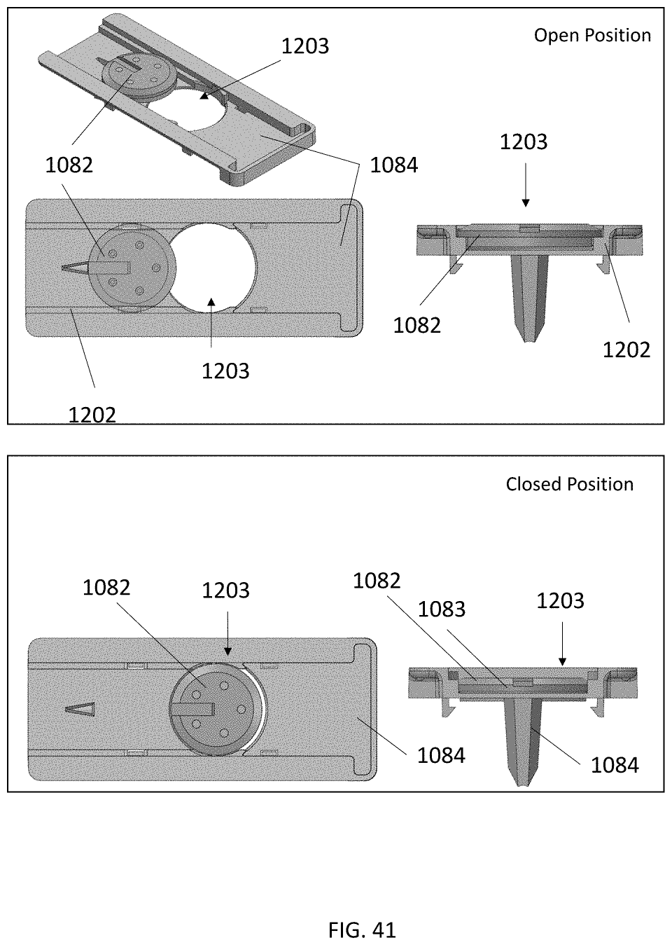

In some embodiments, the slider 1081 is configured as depicted in FIGS. 42 and 43. In some embodiments, the slider is configured to move the crush disc 1082 and the elastomer 1083 over the laminate 1085 and apply force to express plasma. In some embodiments, rails 1300 on either side are configured to interface with the outer rails 1201 of the enclosure lid 1084. In some embodiments, grooves 1301 cut into these rails are configured to interface with bumpers 1204 on the enclosure lid to help hold the device in the open position and prevent accidental actuation. In some embodiments, a ramp 1302 is configured to apply force to the top of the crush disc 1082 once actuated. In some embodiments, the height of the ramp is dictated by the force required to express plasma. In some embodiments, the slider 1081 comprises tabs 1303 configured to hold the crush disc 1082 in the proper orientation. In some embodiments, the tabs 1303 improve sealing of the elastomer 1083 to the laminate 1085.

In some embodiments, the slider 1081 is configured to interface with the enclosure lid 1084, such as depicted in FIG. 42. In some embodiments, when the slider 1081 and the enclosure lid 1084 are in the open position, as depicted in FIG. 43, the interface of the bumper and the grooves are configured to secure the slider in the open position. In some embodiments, the alignment tabs 1303 are configured to interfere with the enclosure lid 1084 to prevent the user from sliding the slider 1081 father than necessary.

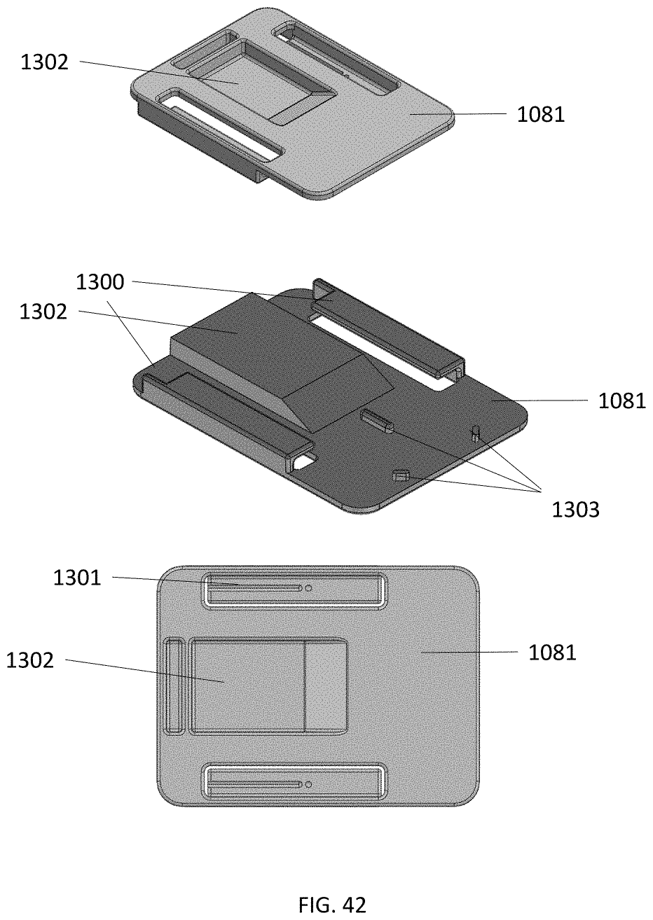

In some embodiments, the slider 1081, crush disc 1082, elastomer 1083 and enclosure lid 1084 interact as depicted in FIG. 44A-44D. In some embodiments, the alignment tabs 1303 keep the crush disc 1082 in the proper position. In some embodiments, the slide ramp applies pressure to the top of crush disc 1082 in closed position 1305. In some embodiments, the slider ramp 1302 pushes the crush disc 1082 during actuation as depicted in FIG. 44A-44D. In some embodiments, the alignment tabs 1303 are configured to apply pressure to the top of the crush disc 1082 during actuation to ensure the laminate inlet is sealed.

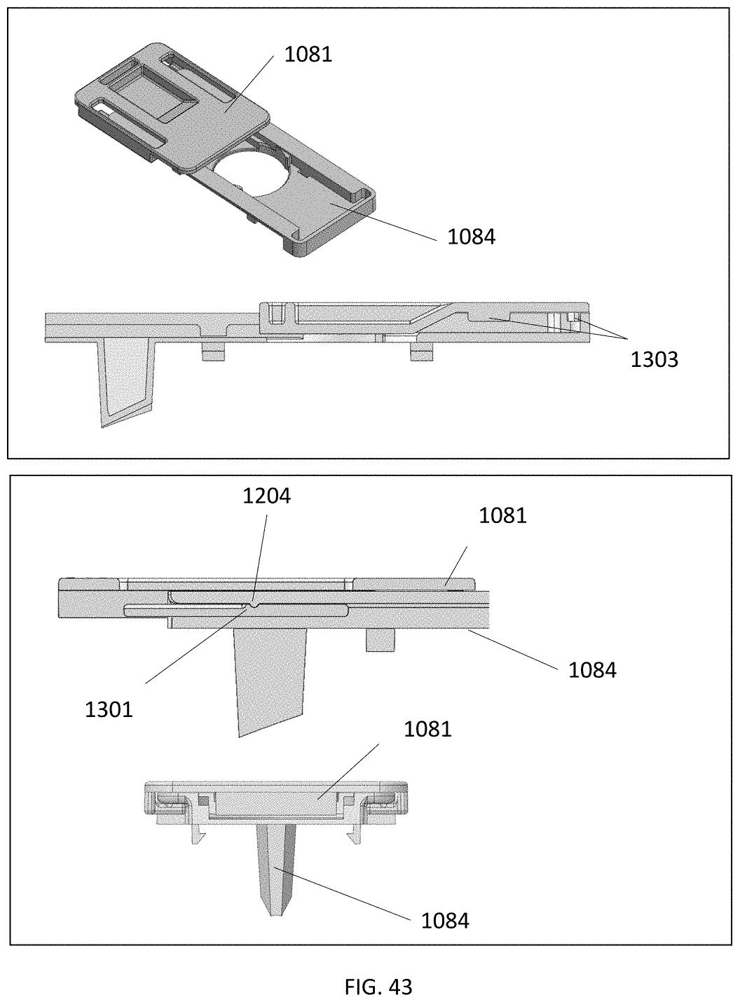

In some embodiments, the device comprises a shipping container, such as depicted in FIG. 45. In some embodiments, the shipping container comprises a shipping container lid 1088 and a shipping container base 1089. In some embodiments, the shipping container is responsible for removing the outlet channel of the laminate from the collection tube. In some embodiments, the shipping container is responsible for closing the lid of the collection tube. In some embodiments, the shipping container is responsible for protecting the device during shipment. In some embodiments, the shipping container base 1089 comprises a closing arm 1305. In some embodiments, when the plasma separation device 1080 is placed in the shipping container, the long, horizontal protrusion 1306 at the top of the closing arm 1305 contacts the outlet channel 1200 of the laminate 1085 and pushes it out from the collection tube 1086. In some embodiments, the vertical portion 1307 of the closing arm then forces the lid of the collection tube closed, sealing the plasma inside for shipment. In some embodiments, the shipping container lid 1088 comprises snap features 1308 that are configured to engage with snap features 1309 on the shipping container base 1089 and are responsible for preventing accidental movement of the slider during shipping.

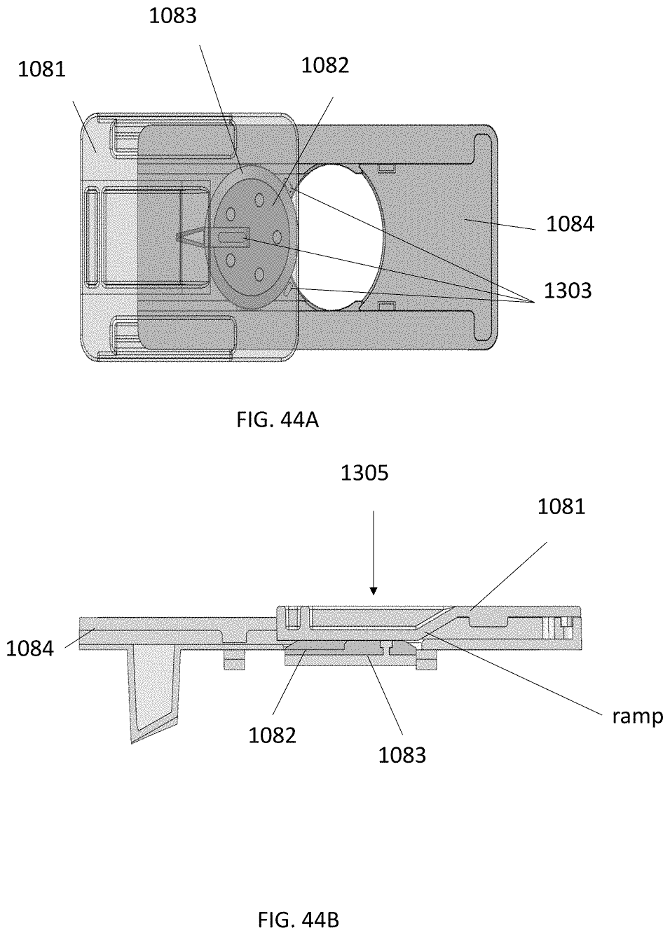

In some embodiments, the device is in the open position, as depicted in FIG. 46, the slider 1081, crush disc 1082, and elastomer 1083 are set back from the laminate 1085 so the user can drop blood into the inlet hole 1098 of the laminate 1085. In some embodiments, the outlet channel 1200 of the laminate is inside the collection tube 1086. In some embodiments, the user receives the device in the open position.

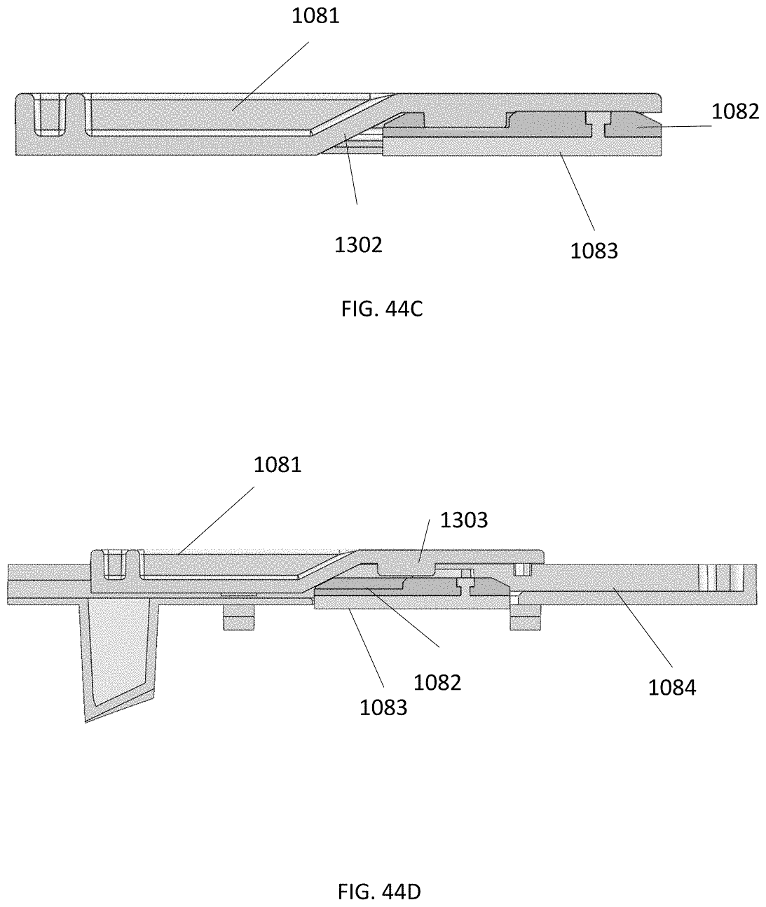

In some embodiments, once blood has been deposited into the laminate, the user will move the slider mechanism to the closed position, as depicted in FIG. 47. In some embodiments, as the slider 1081 moves along the outer rails 1201 of the enclosure lid 1084, it pushes the crush disc 1082 along the inner rails 1202. In some embodiments, once the crush disc 1082 and elastomer 1083 reach the laminate cutout 1203 on the enclosure lid, they drop down on top of the laminate 1085. In some embodiments, as the slider 1081 is progressed past the laminate cutout 1203 the slider ramp 1302 applies force to the crush disc 1082, expressing plasma through the laminate 1085 and into the collection tube 1086. In some embodiments, alignment tabs 1303 on the slider 1081 will eventually contact the enclosure lid 1084, preventing any more forward motion to indicate to the user the device is in the closed position. In some embodiments, after being closed for about 1 minute, all plasma has been expressed into the collection tube 1086. In some embodiments, the collection tube lid 1400 is still open at this time.

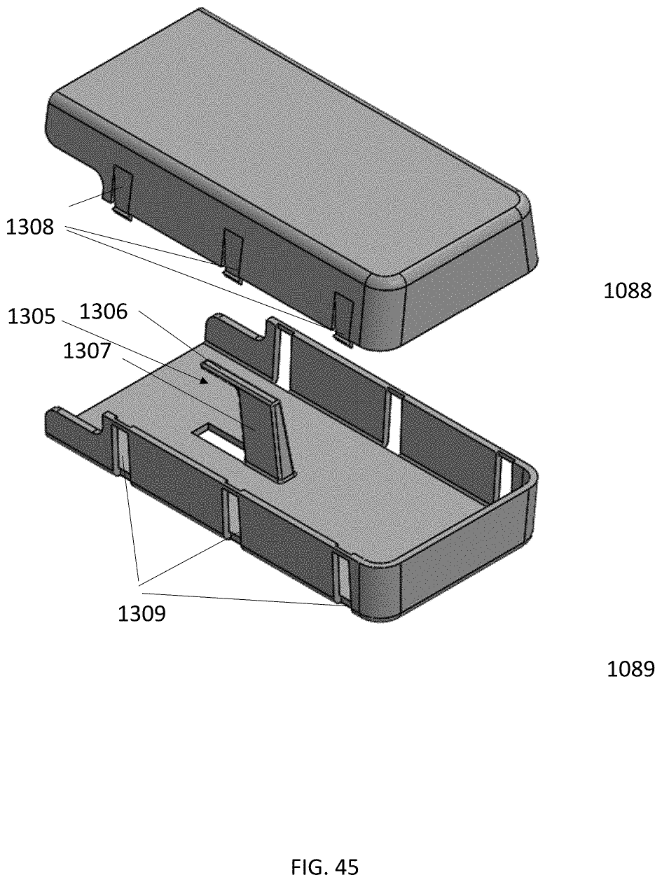

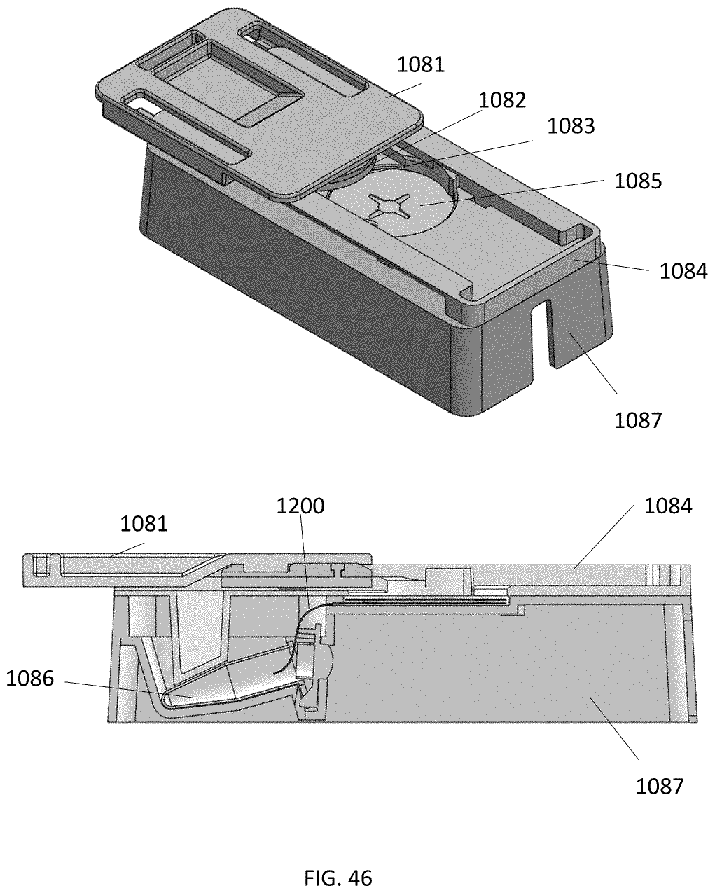

In some embodiments, once the device is in the closed position and plasma is been expressed into the collection tube 1086, the device may be placed in the shipping container 1088 and 1089, as depicted in FIG. 48. In some embodiments the process of placing the device 1080 in the shipping container 1088, 1089 is as depicted in FIG. 49. In some embodiments, the device 1080 is partially inserted in the shipping container 1088, 1089 and the laminate channel 1200 is still in the collection tube 1086 with the collection tube lid 1400 still open. In some embodiments, when the device 1080 is partially inserted in shipping container 1088, 1089, the laminate channel 1200 is removed from the collection tube 1086 and the collection tube lid 1400 still open. In some embodiments, the closing arm 1305 pushes the outlet channel 1200 of the laminate 1085 out of the collection tube 1086 and closes the collection tube lid 1400. In some embodiments, when the device fully inserted in shipping container 1088, 1089, the laminate channel 1200 has been removed from collection tube 1086 and the collection tub lid 1400 is now closed. In some embodiments, the device is fully protected within the shipping container, preventing accidental movement of the slider, which could cause blood leakage and the device is ready to be shipped.

### Blood Plasma Separation Device Using Centrifugation

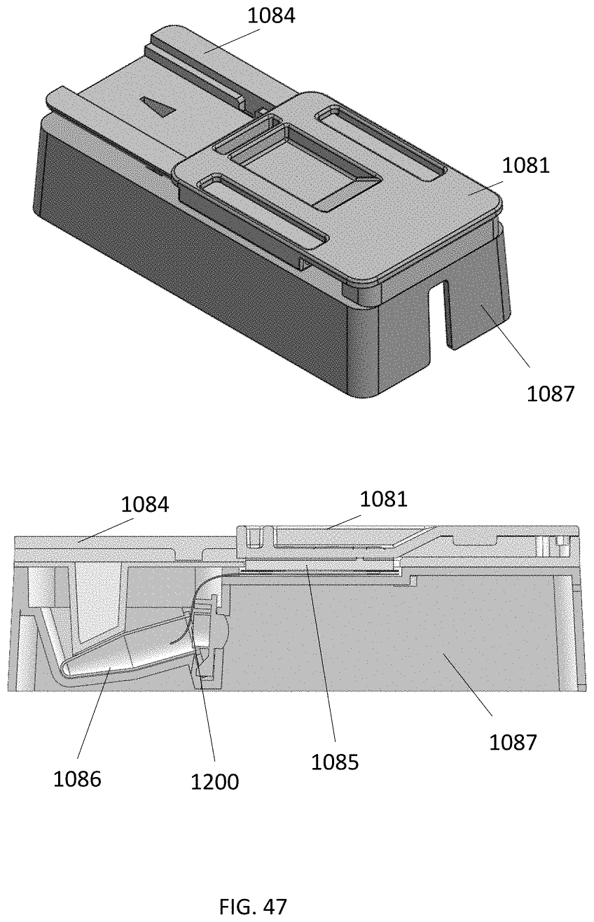

In some embodiments, the disclosure provided herein includes a device for collecting blood plasma from a blood sample through applying centripetal force to the blood sample, thereby pushing the blood sample into a filter or membrane. In some embodiments, the device may comprise: (a) a sample inlet 900 configured to introduce a blood sample into the device 901; (b) a filter or membrane 904 configured to separate plasma from the whole blood; (c) a collection vessel 102 configured to collect the plasma; and (d) an adapter (e.g., FIG. 50A and FIG. 50B) configured to attach the device 901 to a centrifuge tube. In some instances, the adapter may be configured to attach the device to a centrifuge tube, to a plate, or interfaces to the centrifuge without an additional centrifuge tube.

In some embodiments, the filter or membrane 904, may comprise a vertical filter (e.g., sample moves in a gravitational direction). In some instances, a vertical filter comprises a filter matrix. In some instances, the filter matrix of the vertical filter comprises a pore with a pore size that is prohibitive for cells to pass through, while plasma can pass the filter matrix uninhibited. In some instances, the filter matrix comprises a membrane that is especially suited for this application because it combines a large pore size at the top with a small pore size at the bottom of the filter, which leads to very gentle treatment of the cells preventing cell degradation during the filtration process. Alternatively, multiple filters or membranes may be used having different pore sizes. In some instances, the device may comprise two or more filters or membranes, described elsewhere herein, each comprising a plurality of pores with an average pore size that is different for each filter or membrane.

In some instances, the filter or membrane separates substances in the sample based on size, for example, the filter or membrane has a pore size that excludes a cell but is permeable to cell-free nucleic acids. Therefore, the plasma or serum can move more rapidly than a blood cell through the filter or membrane, and the plasma or serum containing any cell-free nucleic acids permeates the holes of the filter or membrane. In some instances, the cell that is slowed and/or trapped in the filter or membrane is a red blood cell, a white blood cell, or a platelet.

In some instances, the filter or membrane is capable of slowing and/or trapping a cell without damaging the cell, thereby avoiding the release of cell contents including cellular nucleic acids and other proteins or cell fragments that could interfere with subsequent evaluation of the cell-free nucleic acids. In some instances, at least 95%, at least 98%, at least 99%, or up to 100% of the cells in a blood sample remain intact when trapped in the filter or membrane. In addition to or independently of size separation, the filter or membrane can trap or separate unwanted substances based on a cell property other than size, for example, the filter or membrane can comprise a binding moiety that binds to a cell surface marker. In some instances, the binding moiety is an antibody or antigen binding antibody fragment. In some instances, the binding moiety is a ligand or receptor binding protein for a receptor on a blood cell or microvesicle.

Examples of filter or membrane materials used in the device to remove cells include, but are not limited to, polyvinylidene difluoride, polytetrafluoroethylene, acetylcellulose, nitrocellulose, polycarbonate, polyethylene terephthalate, polyethylene, polypropylene, glass fiber, borosilicate, vinyl chloride, silver. Suitable filter or membranes may be characterized as preventing passage of cells. In some instances, the filter or membrane is a hydrophobic filter, for example a glass fiber filter, a composite filter, for example Cytosep (e.g., Ahlstrom Filtration or Pall Specialty Materials, Port Washington, N.Y.), or a hydrophilic filter, for example cellulose (e.g., Pall Specialty Materials).

In some instances, the filter or membrane is characterized by at least one pore size. In some instances, the device comprises multiple filters and/or membranes, wherein the pore size of at least a first filter or membrane differs from a second filter or membrane. In some instances, at least one pore size of at least one filter/membrane is about 0.05 microns to about 10 microns. In some instances, the pore size is about 0.05 microns to about 8 microns. In some instances, the pore size is about 0.05 microns to about 6 microns. In some instances, the pore size is about 0.05 microns to about 4 microns. In some instances, the pore size is about 0.05 microns to about 2 microns. In some instances, the pore size is about 0.05 microns to about 1 micron. In some instances, at least one pore size of at least one filter/membrane is about 0.1 microns to about 10 microns. In some instances, the pore size is about 0.1 microns to about 8 microns. In some instances, the pore size is about 0.1 microns to about 6 microns. In some instances, the pore size is about 0.1 microns to about 4 microns. In some instances, the pore size is about 0.1 microns to about 2 microns. In some instances, the pore size is about 0.1 microns to about 1 micron.

The device may be configured to separate and collect a volume of plasma from the blood sample that is greater than about 25% of an input or starting volume of the blood sample. For example, the device may be configured to separate and collect a volume of plasma from the blood sample that is greater than about 30%, greater than about 35%, greater than about 40%, or more of an input or starting volume of the blood sample.

The device may be configured to separate and collect an amount of plasma from the blood sample that is at least about 50% of the total amount of recoverable plasma present in the blood sample. For example, the device may be configured to separate and collect an amount of plasma from the blood sample that is at least about 55%, at least about 60%, at least about 65%, at least about 70%, at least about 75%, at least about 80%, at least about 85%, at least about 90%, at least about 95%, or greater of the total amount of recoverable plasma present in the blood sample.

The devices may be configured to separate and collect plasma from an ultra-low volume of a blood sample. In some instances, the devices disclosed herein are used with not more than 25 μL of the blood sample. In some instances, devices disclosed herein are used with not more than 50 μL of the blood sample. In some instances, devices disclosed herein are used with not more than 75 μL of the blood sample. In some instances, devices disclosed herein are used with not more than 100 μL of the blood sample. In some instances, devices disclosed herein are used with not more than 125 μL of the blood sample. In some instances, devices disclosed herein are used with not more than 150 μL of the blood sample. In some instances, devices disclosed herein are used with not more than 200 μL of the blood sample. In some instances, devices disclosed herein are used with not more than 300 μL of the blood sample. In some instances, devices disclosed herein are used with not more than 400 μL of the biological fluid sample. In some instances, devices disclosed herein are used with not more than 500 μL of the biological fluid sample. In some instances, devices disclosed herein are used with not more than 1 mL of the biological fluid sample.

In some instances, the devices disclosed herein are used with an ultra-low volume of a blood sample, wherein the ultra-low volume falls within a range of sample volumes. In some instances, the range of sample volumes is about 5 μL to about 1 mL. In some instances, the range of sample volumes is about 5 μL to about 900 μL. In some instances, the range of sample volumes is about 5 μL to about 800 μL. In some instances, the range of sample volumes is about 5 μL to about 700 μL. In some instances, the range of sample volumes is about 5 μL to about 600 μL. In some instances, the range of sample volumes is about 5 μL to about 500 μL. In some instances, the range of sample volumes is about 5 μL to about 400 μL. In some instances, the range of sample volumes is about 5 μL to about 300 μL. In some instances, the range of sample volumes is about 5 μL to about 200 μL. In some instances, the range of sample volumes is about 5 μL to about 150 μL. In some instances, the range of sample volumes is 5 μL to about 100 μL. In some instances, the range of sample volumes is about 5 μL to about 90 μL. In some instances, the range of sample volumes is about 5 μL to about 85 μL. In some instances, the range of sample volumes is about 5 μL to about 80 μL. In some instances, the range of sample volumes is about 5 μL to about 75 μL. In some instances, the range of sample volumes is about 5 μL to about 70 μL. In some instances, the range of sample volumes is about 5 μL to about 65 μL. In some instances, the range of sample volumes is about 5 μL to about 60 μL. In some instances, the range of sample volumes is about 5 μL to about 55 μL. In some instances, the range of sample volumes is about 5 μL to about 50 μL. In some instances, the range of sample volumes is about 15 μLμμ to about 150 μL. In some instances, the range of sample volumes is about 15 μL to about 120 μL. In some instances, the range of sample volumes is 15 μL to about 100 μL. In some instances, the range of sample volumes is about 15 μL to about 90 μL. In some instances, the range of sample volumes is about 15 μL to about 85 μL. In some instances, the range of sample volumes is about 15 μL to about 80 μL. In some instances, the range of sample volumes is about 15 μL to about 75 μL. In some instances, the range of sample volumes is about 15 μL to about 70 μL. In some instances, the range of sample volumes is about 15 μL to about 65 μL. In some instances, the range of sample volumes is about 15 μL to about 60 μL. In some instances, the range of sample volumes is about 15 μL to about 55 μL. In some instances, the range of sample volumes is about 15 μL to about 50 μL.

In some instances, the devices disclosed herein are used with an ultra-low volume of a blood sample, wherein the ultra-low volume is about 100 μL to about 500 μL. In some instances, the devices disclosed herein are used with an ultra-low volume of the blood sample, wherein the ultra-low volume about 100 μL to about 1000 μL. In some instances, the ultra-low volume is about 500 μL to about 1 ml. In some instances, the ultra-low volume is about 500 μL to about 2 ml. In some instances, the ultra-low volume is about 500 μL to about 3 ml. In some instances, the ultra-low volume is about 500 μL to about 5 ml.

The ultra-low volume may be about 1 μL to about 250 μL. The ultra-low volume may be about 5 μL to about 250 μL. The ultra-low volume may be about 10 μL to about 25 μL. The ultra-low volume may be about 10 μL to about 35 μL. The ultra-low volume may be about 10 μL to about 45 μL. The ultra-low volume may be about 10 μL to about 50 μL. The ultra-low volume may be about 10 μL to about 60 μL. The ultra-low volume may be about 10 μL to about 80 μL. The ultra-low volume may be about 10 μL to about 100 μL. The ultra-low volume may be about 10 μL to about 120 μL. The ultra-low volume may be about 10 μL to about 140 μL. The ultra-low volume may be about 10 μL to about 150 μL. The ultra-low volume may be about 10 μL to about 160 μL. The ultra-low volume may be about 10 μL to about 180 μL. The ultra-low volume may be about 10 μL to about 200 μL.

The ultra-low volume may be about 1 μL to about 200 μL. The ultra-low volume may be about 1 μL to about 190 μL. The ultra-low volume may be about 1 μL to about 180 μL. The ultra-low volume may be about 1 μL to about 160 μL. The ultra-low volume may be about 1 μL to about 150 μL. The ultra-low volume may be about 1 μL to about 140 μL. The ultra-low volume may be about 5 μL to about 15 μL. The ultra-low volume may be about 5 μL to about 25 μL. The ultra-low volume may be about 5 μL to about 35 μL. The ultra-low volume may be about 5 μL to about 45 μL. The ultra-low volume may be about 5 μL to about 50 μL. The ultra-low volume may be about 5 μL to about 60 μL. The ultra-low volume may be about 5 μL to about 70 μL. The ultra-low volume may be about 5 μL to about 80 μL. The ultra-low volume may be about 5 μL to about 90 μL. The ultra-low volume may be about 5 μL to about 100 μL. The ultra-low volume may be about 5 μL to about 125 μL. The ultra-low volume may be about 5 μL to about 150 μL. The ultra-low volume may be about 5 μL to about 175 μL. The ultra-low volume may be about 5 μL to about 200 μL.

In some instances, the filter or membrane may be configured to separate plasma from an input volume of no more than about 1 milliliter (mL), no more than 500 microliters (μL), no more than 100 μL, no more than 50 μL, no more than about 25 microliters (μL), or less of the blood sample. In some cases, the filter or membrane is configured to remove cells, cell fragments, microvesicles, or any combination thereof from the whole blood sample. In some cases, the filter or membrane comprises a plurality of pores. In some instances, the plurality of pores located at a first side of the filter or membrane have an average pore size that is greater than an average pore size of a plurality of pores located at a second side of the filter or membrane.

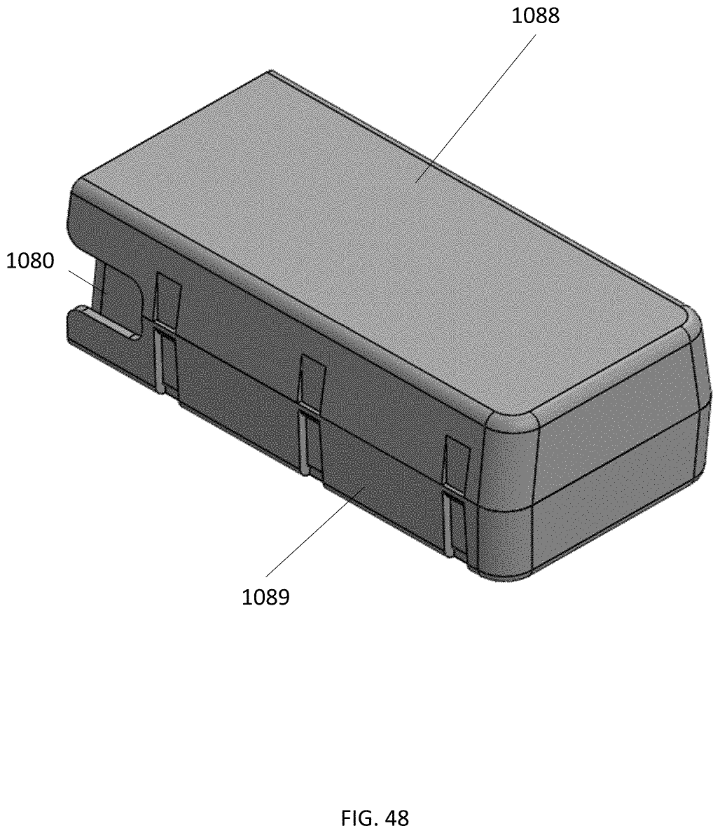

In some cases, the device 901, may mount onto a collection vessel (e.g., PCR or Eppendorf tube(s)) via a feature that may include a snap fit mechanism that allows for the plasma or other components separated by the device to be directed into the collection vessel (e.g., PCR or Eppendorf tube(s)). In some instances, the device may comprise a laminate comprising a plurality of layers as seen in FIG. 51A and FIG. 51B. In some cases, the plurality of layers of the laminate may comprise: (a) a blood inlet chamber 900, where the blood inlet layer may accept a sample of blood; (b) a plurality of adhesive layers 902 to seal the chamber; (c) a membrane or filter 904 configured to pass blood plasma while retaining one or more blood components other than plasma; (d) a membrane support layer 910; (e) a transfer channel layer; and a collection interface 914. In some cases, the collection interface may further be in fluidic communication with a needle or cannula 916, where the needle or cannula 916 may deposit the collected blood plasma into the collection vessel 102.

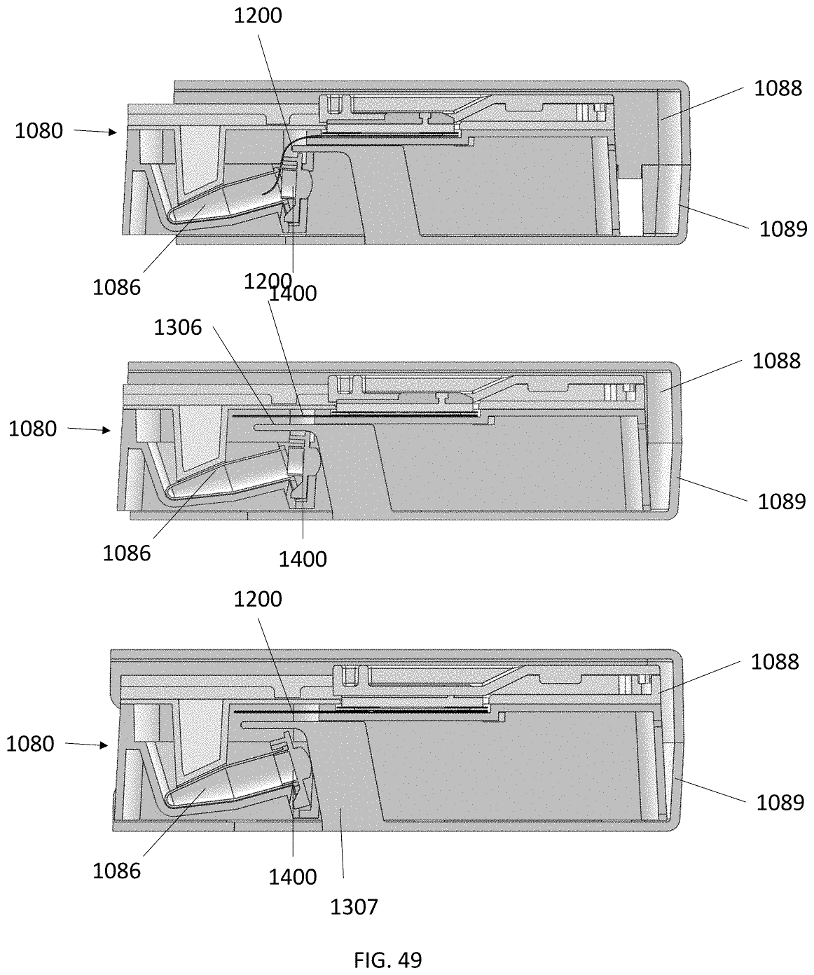

In some instances, the device may comprise a subset of layers of the plurality of layers of the laminate, as seen in FIG. 52. In some instances, the device may be configured to extract small volumes of blood plasma. In some embodiments, the laminate 915, 901 may be attached to a collection vessel 102 filled with a blood sample and placed into a centrifuge adapter, e.g., as seen in FIG. 50A and FIG. 50B. The collection vessel centrifuge adapter may provide mechanical coupling between a standard 50 mL centrifuge tube holder rotor and the device.

### Methods

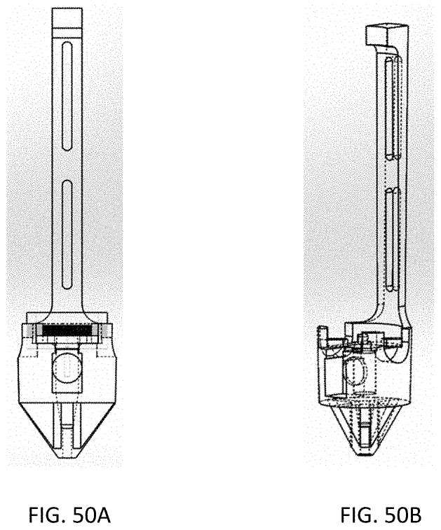

In some cases, the methods described herein may comprise a method of collecting and/or extracting blood plasma 1000 e.g., with a device as described elsewhere herein, e.g., as seen in FIG. 53. In some instances, the method may comprise (a) providing or obtaining a blood sample obtained from an individual 1002; (b) applying a positive pressure to a starting volume of the blood sample such that the blood sample is pushed into a filter or a membrane, wherein the starting volume of the blood sample is not more than about 1 milliliter (mL) 1004; (c) filtering the blood sample through the filter or the membrane to separate plasma from the blood sample 1006; and (d) collecting the plasma in a collection vessel 1008.

In some instances, the method may result in an enrichment of cell-free nucleic acids. In some cases, the methods involve separating a volume of plasma from the blood sample that is greater than about 25% of an input or starting volume of the blood sample. For example, the methods may involve separating a volume of plasma from the blood sample that is greater than about 30%, greater than about 35%, greater than about 40%, greater than about 50%, or more of an input or starting volume of the blood sample.

In some cases, the methods involve separating an amount of plasma from the blood sample that is at least about 50% of the total amount of recoverable plasma present in the blood sample. For example, the methods involve separating an amount of plasma from the blood sample that is at least about 55%, at least about 60%, at least about 65%, at least about 70%, at least about 75%, at least about 80%, at least about 85%, at least about 90%, at least about 95%, or greater of the total amount of recoverable plasma present in the blood sample.

In some cases, the methods may involve separating plasma from an ultra-low volume of a blood sample. In some instances, the methods are used with not more than 50 μL of the blood sample. In some instances, the methods are used with not more than 75 μL of the blood sample. In some instances, the methods are used with not more than 100 μL of the blood sample. In some instances, the methods are used with not more than 125 μL of the blood sample. In some instances, the methods are used with not more than 150 μL of the blood sample. In some instances, the methods are used with not more than 200 μL of the blood sample. In some instances, the methods are used with not more than 300 μL of the blood sample. In some instances, the methods are used with not more than 400 μL of the biological fluid sample. In some instances, the methods are used with not more than 500 μL of the biological fluid sample. In some instances, the methods are used with not more than 1 mL of the biological fluid sample.

In some instances, the methods are used with an ultra-low volume of a blood sample, wherein the ultra-low volume falls within a range of sample volumes. In some instances, the range of sample volumes is about 5 μL to about 1 mL. In some instances, the range of sample volumes is about 5 μL to about 900 μL. In some instances, the range of sample volumes is about 5 μL to about 800 μL. In some instances, the range of sample volumes is about 5 μL to about 700 μL. In some instances, the range of sample volumes is about 5 μL to about 600 μL. In some instances, the range of sample volumes is about 5 μL to about 500 μL. In some instances, the range of sample volumes is about 5 μL to about 400 μL. In some instances, the range of sample volumes is about 5 μL to about 300 μL. In some instances, the range of sample volumes is about 5 μL to about 200 μL. In some instances, the range of sample volumes is about 5 μL to about 150 μL. In some instances, the range of sample volumes is 5 μL to about 100 μL. In some instances, the range of sample volumes is about 5 μL to about 90 μL. In some instances, the range of sample volumes is about 5 μL to about 85 μL. In some instances, the range of sample volumes is about 5 μL to about 80 μL. In some instances, the range of sample volumes is about 5 μL to about 75 μL. In some instances, the range of sample volumes is about 5 μL to about 70 μL. In some instances, the range of sample volumes is about 5 μL to about 65 μL. In some instances, the range of sample volumes is about 5 μL to about 60 μL. In some instances, the range of sample volumes is about 5 μL to about 55 μL. In some instances, the range of sample volumes is about 5 μL to about 50 μL. In some instances, the range of sample volumes is about 15 μLμμ to about 150 μL. In some instances, the range of sample volumes is about 15 μL to about 120 μL. In some instances, the range of sample volumes is 15 μL to about 100 μL. In some instances, the range of sample volumes is about 15 μL to about 90 μL. In some instances, the range of sample volumes is about 15 μL to about 85 μL. In some instances, the range of sample volumes is about 15 μL to about 80 μL. In some instances, the range of sample volumes is about 15 μL to about 75 μL. In some instances, the range of sample volumes is about 15 μL to about 70 μL. In some instances, the range of sample volumes is about 15 μL to about 65 μL. In some instances, the range of sample volumes is about 15 μL to about 60 μL. In some instances, the range of sample volumes is about 15 μL to about 55 μL. In some instances, the range of sample volumes is about 15 μL to about 50 μL.

In some instances, the methods are used with an ultra-low volume of a blood sample, wherein the ultra-low volume is about 100 μL to about 500 μL. In some instances, the methods are used with an ultra-low volume of the blood sample, wherein the ultra-low volume about 100 μL to about 1000 μL. In some instances, the ultra-low volume is about 500 μL to about 1 ml. In some instances, the ultra-low volume is about 500 μL to about 2 ml. In some instances, the ultra-low volume is about 500 μL to about 3 ml. In some instances, the ultra-low volume is about 500 μL to about 5 ml.

The ultra-low volume may be about 1 μL to about 250 μL. The ultra-low volume may be about 5 μL to about 250 μL. The ultra-low volume may be about 10 μL to about 25 μL. The ultra-low volume may be about 10 μL to about 35 μL. The ultra-low volume may be about 10 μL to about 45 μL. The ultra-low volume may be about 10 μL to about 50 μL. The ultra-low volume may be about 10 μL to about 60 μL. The ultra-low volume may be about 10 μL to about 80 μL. The ultra-low volume may be about 10 μL to about 100 μL. The ultra-low volume may be about 10 μL to about 120 μL. The ultra-low volume may be about 10 μL to about 140 μL. The ultra-low volume may be about 10 μL to about 150 μL. The ultra-low volume may be about 10 μL to about 160 μL. The ultra-low volume may be about 10 μL to about 180 μL. The ultra-low volume may be about 10 μL to about 200 μL.

The ultra-low volume may be about 1 μL to about 200 μL. The ultra-low volume may be about 1 μL to about 190 μL. The ultra-low volume may be about 1 μL to about 180 μL. The ultra-low volume may be about 1 μL to about 160 μL. The ultra-low volume may be about 1 μL to about 150 μL. The ultra-low volume may be about 1 μL to about 140 μL. The ultra-low volume may be about 5 μL to about 15 μL. The ultra-low volume may be about 5 μL to about 25 μL. The ultra-low volume may be about 5 μL to about 35 μL. The ultra-low volume may be about 5 μL to about 45 μL. The ultra-low volume may be about 5 μL to about 50 μL. The ultra-low volume may be about 5 μL to about 60 μL. The ultra-low volume may be about 5 μL to about 70 μL. The ultra-low volume may be about 5 μL to about 80 μL. The ultra-low volume may be about 5 μL to about 90 μL. The ultra-low volume may be about 5 μL to about 100 μL. The ultra-low volume may be about 5 μL to about 125 μL. The ultra-low volume may be about 5 μL to about 150 μL. The ultra-low volume may be about 5 μL to about 175 μL. The ultra-low volume may be about 5 μL to about 200 μL.

In some cases, the volume of the plasma collected may be greater than about 30%, greater than about 35%, or greater than about 40% of the starting volume of the whole blood sample. In some cases, the plasma may comprise cell-free nucleic acids. In some cases, the plasma may be substantially free of cellular nucleic acids. In some instances, the plasma may be substantially free of cells, cell fragments, microvesicles, or any combination thereof. In some instances, the method may be performed in 1 minute or less. In some instances, the method may not result in substantial lysis or disruption of white blood cells. In some cases, the positive pressure may be selected such that hemolysis of red blood cells may occur, but white blood cells are not lysed or disrupted. In some cases, the positive pressure may be an amount of less than about 4 pounds per square inch (psi). In some cases, the positive pressure may be an amount of about 4 to about 11 pounds per square inch (psi). In some cases, the positive pressure may be an amount greater than about 11 psi (e.g., greater than about 15 psi, greater than about 20 psi, or greater).

In some cases, the blood sample may be capillary blood. In some cases, the blood samples may be obtained via a finger prick. In some cases, a volume of plasma collected may be greater than a volume of plasma collected using an equivalent method using negative pressure or vacuum. In some instances, a volume of plasma collected may be at least about 10%, at least about 25%, at least about 50%, at least about 75%, at least about 100%, or greater than a volume of plasma collected using an equivalent method using negative pressure or vacuum.

In some cases, the collected plasma comprises cell-free nucleic acids. In some cases, the collected plasma is enriched for cell-free nucleic acids. The cell-free nucleic acids may be deoxyribonucleic acids (cfDNA). In some instances, the cell-free nucleic acids may comprise cell-free nucleic acids from a tumor. In some cases, the cell-free nucleic acids may comprise cell-free nucleic acids from a fetus. In some instances, the cell-free nucleic acids may comprise cell-free nucleic acids from a transplanted tissue or organ. In some cases, the cell-free nucleic acids may comprise from about 10⁴ to about 10⁹ cell-free nucleic acid molecules. The collected plasma may be used in any number of downstream applications.

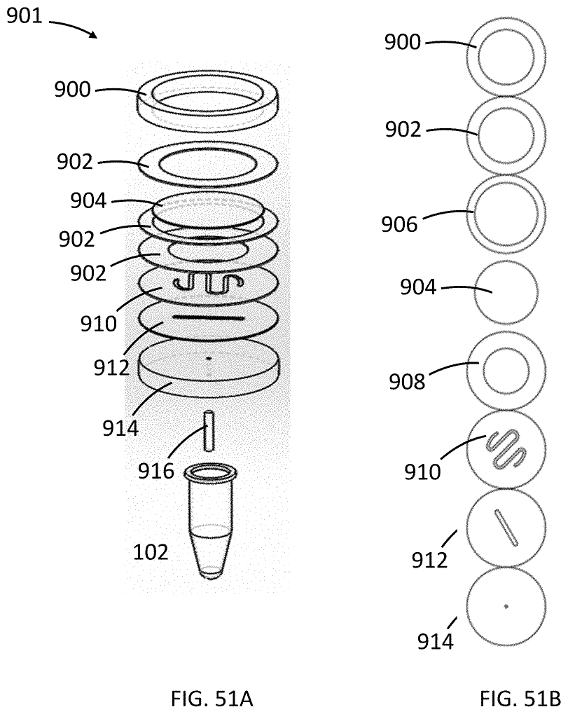

In another aspect, the methods 1010 (e.g., as seen in FIG. 54) described herein may comprise (a) providing or obtaining a blood sample obtained from an individual 1012; (b) centrifuging the blood sample such that the blood sample is forced or pushed into a filter or a membrane 1014; (c) filtering the blood sample through the filter or the membrane to separate plasma from the blood sample 1016; and (d) collecting the plasma in a collection vessel 1018. In some cases, a volume of the plasma is greater than about 25% of an input volume of the blood sample. In some cases, the blood sample may be obtained from the individual by a finger prick. In some cases, the blood sample may be obtained from the individual by a transdermal puncture device. In some instances, the blood sample is or comprises whole blood or one or more blood components. In some instances, the method may be performed in a laboratory setting.

In some instances, the volume of the plasma may be greater than about 30%, greater than about 35%, or greater than about 40%, or greater than about 50% of the starting volume of the blood sample. In some cases, the input volume of the blood sample may not be more than about 500 microliters (μL), not more than about 250 μL, not more than about 150 μL, not more than about 100 μL, not more than about 80 μL, not more than about 60 μL, not more than about 40 μL, or not more than about 25 μL. In some instances, the volume of plasma collected may be greater than a volume of plasma collected using an equivalent method using a centrifuge without the use of the filter or membrane. In some cases, a volume of plasma collected may be at least about 10%, at least about 25%, at least about 50%, at least about 75%, at least about 100%, or greater than a volume of plasma collected using an equivalent method using a centrifuge without the use of the filter or membrane.

In some embodiments, the plasma may comprise cell-free nucleic acids. In some cases, the plasma is substantially free of cellular nucleic acids. In some instances, the method may result in an enrichment of cell-free nucleic acids. In some cases, the plasma is substantially free of, or has a reduction in a population of cells, cell fragments, microvesicles, or any combination thereof. In some cases, the cellular nucleic acids are reduced in the plasma. In some instances, the whole blood sample is capillary blood. In some cases, the method may not result in substantial lysis or disruption of white blood cells. In some cases, the cell-free nucleic acids may comprise deoxyribonucleic acid. In some instances, the cell-free nucleic acid may comprise cell-free nucleic acids from a tumor. In some cases, the cell-free nucleic acids may comprise cell-free nucleic acids from a fetus. In some instances, the cell-free nucleic acids may comprise cell-free nucleic acids from a transplanted tissue or organ. In some cases, the cell-free nucleic acids may comprise from about 10⁴ to about 10⁹ cell-free nucleic acid molecules.

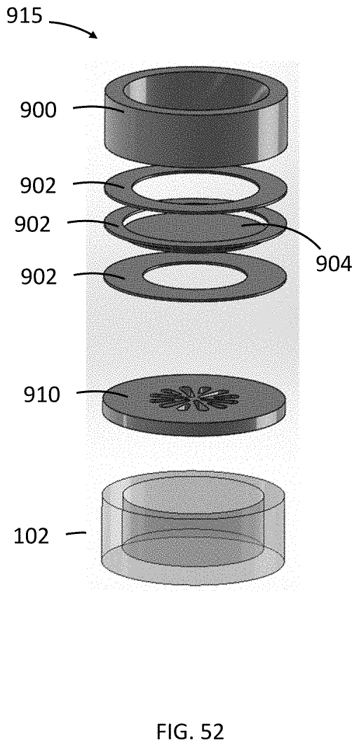

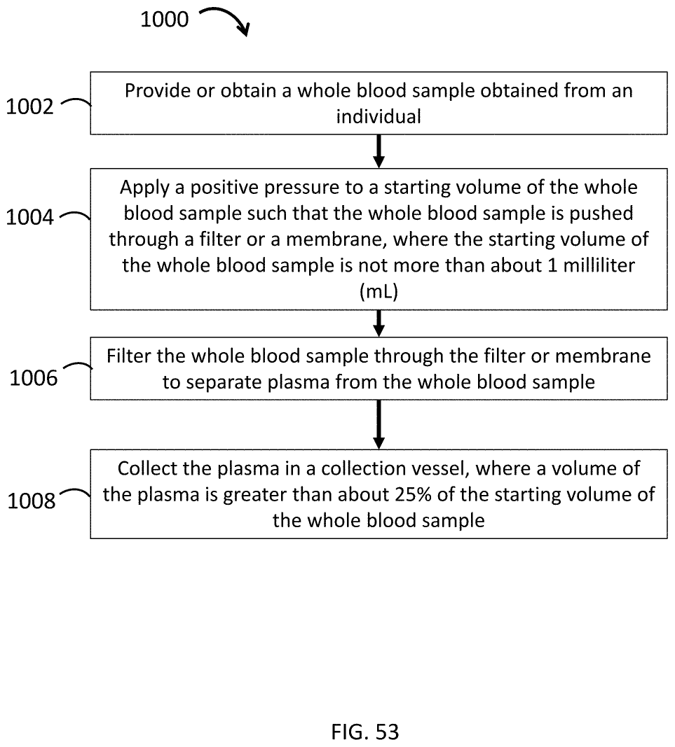

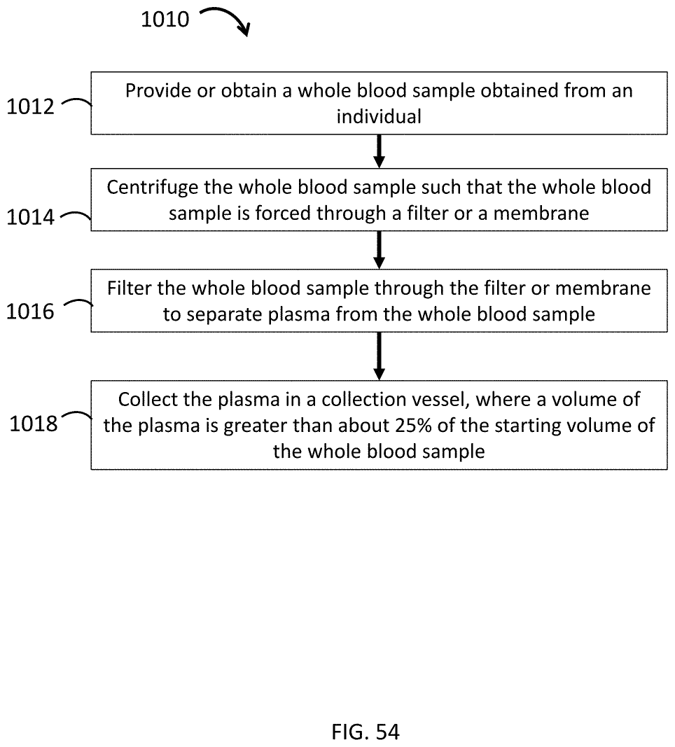

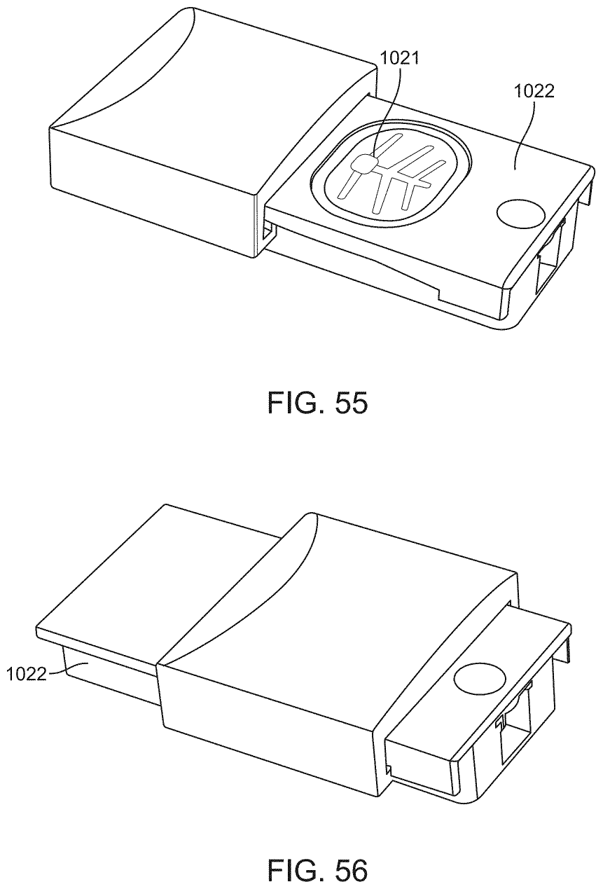

In some embodiments, the methods (e.g., as seen in FIG. 55-58) described herein may comprise (a) the user inputting blood from finger into inlet hole 1021 (b) the user pushing slider 1022 until it stops and (c) the user sliding on the shipping sleeve 1023.

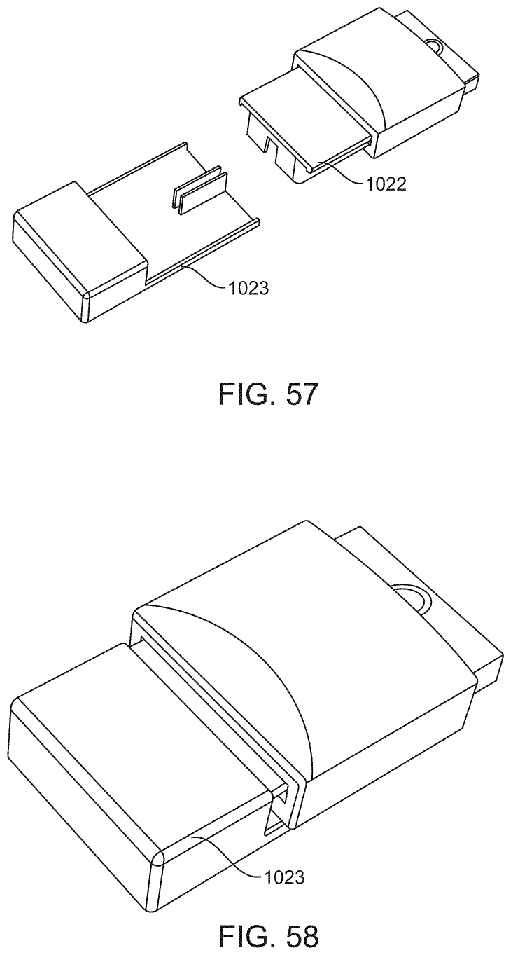

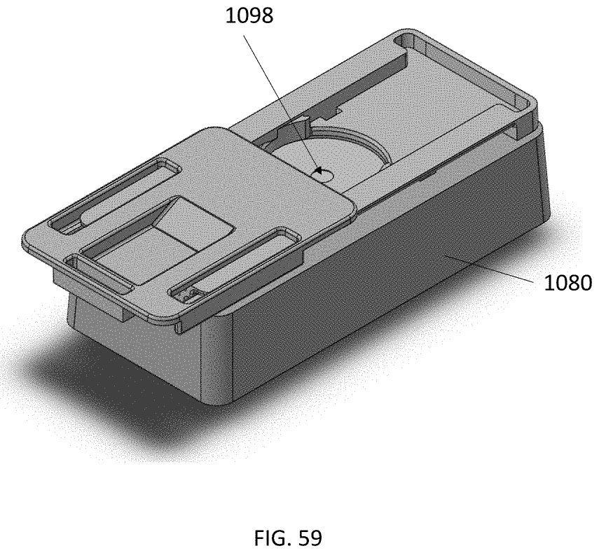

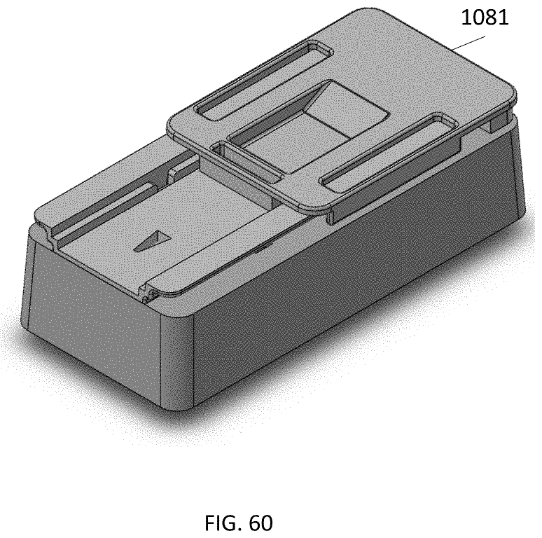

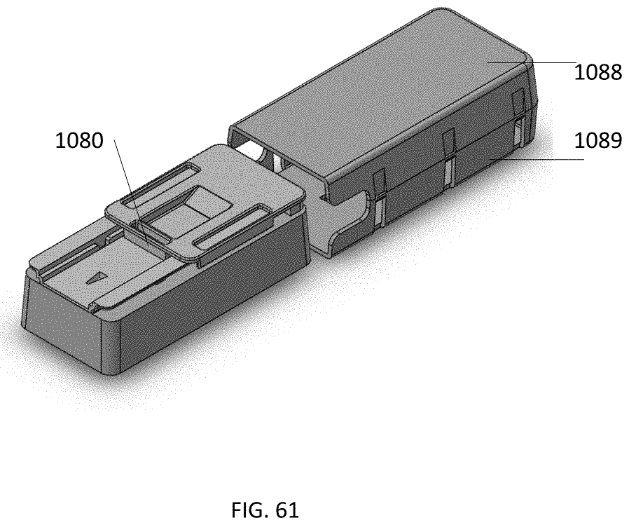

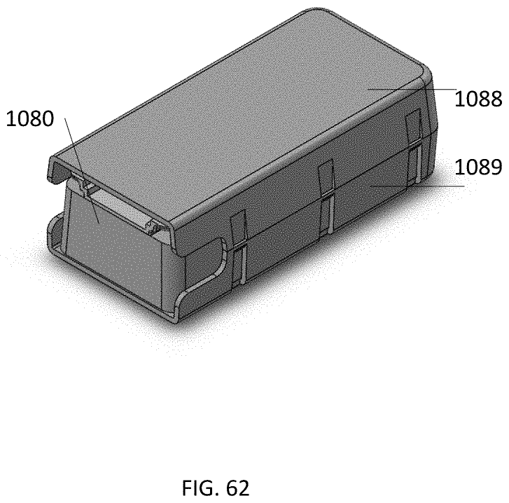

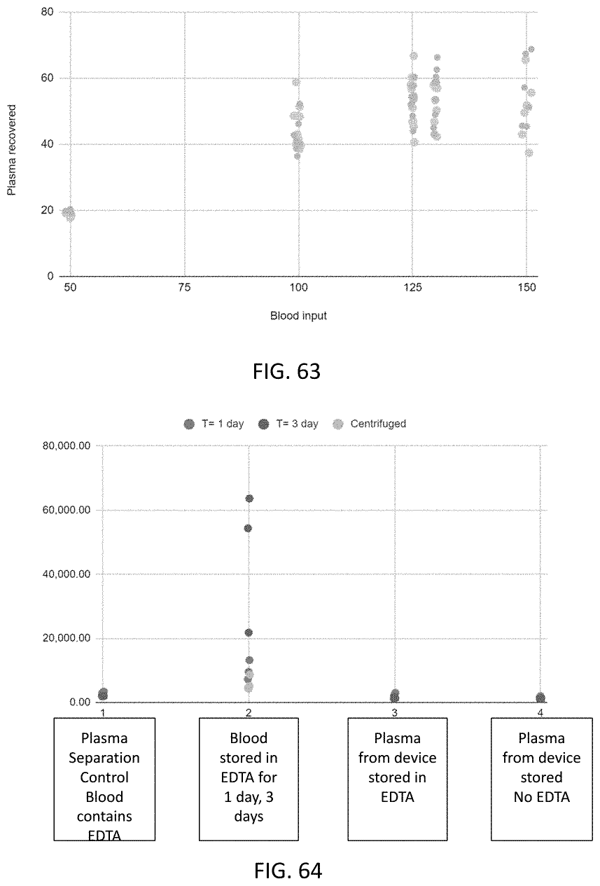

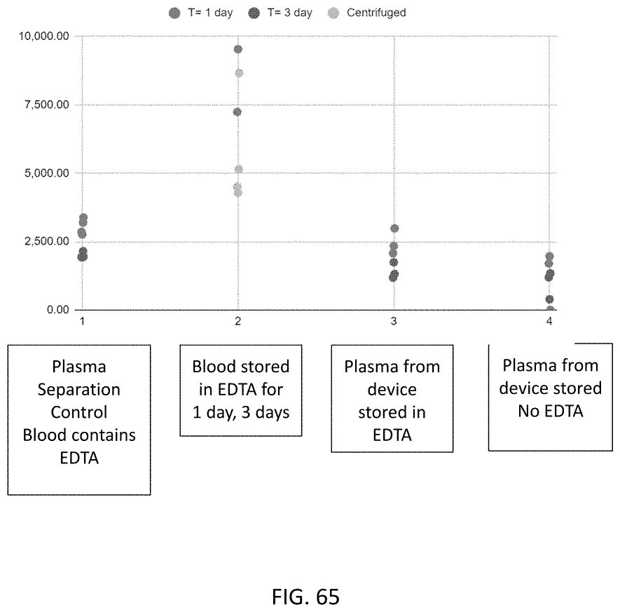

In some embodiments, the methods (e.g., as seen in FIGS. 59-62), may comprise the user input blood from the finger into the inlet hole 1098 in the open position of the device 1080 (FIG. 59), the user pushing the slider 1081 until it stops (FIG. 60), the user obtaining the shipping container 1088, 1089 (FIG. 61), and the user installing the shipping container 1088, 1089 onto the device 1080 (FIG. 62).

### Applications

Devices, and methods disclosed herein may be used to test for, detect, and/or monitor an immune disorder or autoimmune disorder in a subject. Autoimmune and immune disorders include, but are not limited to, type 1 diabetes, rheumatoid arthritis, psoriasis, multiple sclerosis, lupus, inflammatory bowel disease, Addison's Disease, Graves Disease, Crohn's Disease and Celiac disease.

Devices and methods disclosed herein may be used to test for, detect, and/or monitor a disease or condition that is associated with aging of a subject. Disease and conditions associated with aging include, but are not limited to, cancer, osteoporosis, dementia, macular degeneration, metabolic conditions, and neurodegenerative disorders.

Devices and methods disclosed herein may be used to test for, detect, and/or monitor a blood disorder. Non-limiting examples of blood disorders are anemia, hemophilia, blood clotting and thrombophilia. For example, detecting thrombophilia may comprise detecting a polymorphism present in a gene selected from Factor V Leiden (FVL), prothrombin gene (PT G20210A), and methylenetetrahydrofolate reductase (MTHFR).

Devices and methods disclosed herein may be used to test for, detect, and/or monitor a neurological disorder or a neurodegenerative disorder in a subject. Non-limiting examples of neurodegenerative and neurological disorders are Alzheimer's disease, Parkinson's disease, Huntington's disease, Spinocerebellar ataxia, amyotrophic lateral sclerosis (ALS), motor neuron disease, chronic pain, and spinal muscular atrophy. Devices and methods disclosed herein may be used to test for, detect, and/or monitor a psychiatric disorder in a subject and/or a response to a drug to treat the psychiatric disorder.

Devices and methods disclosed herein may be used to test for, detect, and/or monitor a metabolic condition or disease. Metabolic conditions and diseases include, but are not limited to obesity, a thyroid disorder, hypertension, type 1 diabetes, type 2 diabetes, non-alcoholic steatohepatitis, coronary artery disease, and atherosclerosis.

Devices and methods disclosed herein may be used to test for, detect, and/or monitor an allergy or intolerance to a food, liquid or drug. By way of non-limiting example, a subject can be allergic or intolerant to lactose, wheat, soy, dairy, caffeine, alcohol, nuts, shellfish, and eggs. A subject could also be allergic or intolerant to a drug, a supplement or a cosmetic. In some instances, methods comprise analyzing genetic markers that are predictive of skin type or skin health.

In some instances, the condition is associated with an allergy. In some instances, the subject is not diagnosed with a disease or condition, but is experiencing symptoms that indicate a disease or condition is present. In other instances, the subject is already diagnosed with a disease or condition, and the devices and methods disclosed herein are useful for monitoring the disease or condition, or an effect of a drug on the disease or condition.

Devices and methods disclosed herein may be used to test for, detect, and/or monitor a pregnancy. In some instances, the subject is a pregnant subject. in her first, second, or third trimester of pregnancy.

### Non-Invasive Prenatal Testing

One application for devices and methods disclosed herein is non-invasive prenatal testing (NIPT). The health of the fetus is one of the key concerns of expecting parents after the initial awareness and confirmation of a pregnancy. In addition to other general pregnancy-related health tests, assessment of the risk of fetal chromosomal or genetic aberrations has become a standard of care in the management of pregnancies in many countries. Currently, there are several ways to determine genetic information from the fetus. During the first trimester (week 1 through 12), an ultrasound test for nuchal translucency can reveal if there is a likelihood of a chromosomal abnormality, like trisomy 18 or trisomy 21. In addition, a maternal phlebotomy can be performed to test for levels of pregnancy-associated plasma protein and human chorionic gonadotropin. Elevated levels of these proteins may be indicative of a chromosomal abnormality as well. However, these tests are not conclusive and generally require additional, more invasive testing (e.g., chronic villus sampling (sampling of placental tissue), or amniocentesis (needle penetrates the amniotic sac)) to determine if there is indeed an abnormality. Additional tests can be performed during the second trimester, but typically more testing, additional ultrasounds and an amniocentesis, are required for a more definitive determination.

The foregoing described screening requires medical providers with technical training in clinical settings. Many of these tests are invasive (e.g., amniocentesis), thereby carrying a health risk to the fetus, as well as the mother. Typically, the foregoing described screening is necessary at both trimesters to detect a chromosomal abnormality. Thus, detection of a chromosomal abnormality typically cannot be achieved until the fetus is halfway through gestation using the current methods in the field.

Since the discovery of the presence of circulating cell-free fetal DNA in the blood of pregnant women, prenatal care has seen significant improvements. The presence of fetal DNA circulating in maternal blood has afforded a means to study the genetic make-up of the fetus and identify potential health risks or pregnancy complications without the risk associated with procedures such as chorionic villus sampling and amniocentesis. A number of medically relevant tests that utilize circulating cell-free fetal DNA have been developed, but the most prominent ones are NIPT for fetal chromosomal abnormalities.

Existing NIPT can be categorized into two main categories. They are either targeted assays that only amplify and analyze certain chromosomes or chromosomal regions or they are whole genome assays. Unfortunately, existing NIPT requires venipuncture (e.g., a phlebotomy) to obtain amounts of maternal blood/plasma sufficient to achieve appropriate screening performance. For example, existing NIPT often require collection of as much as 16 ml of blood. Because of the large amounts of blood required in existing NIPT, there are significant restrictions in convenience and access to testing. In addition, sample-handling logistics, as well as testing costs and reagent costs are burdensome.

NIPT has previously been thought of as only being feasible with large amounts of cfDNA copy numbers (genome equivalents) such as those obtained with a phlebotomy (e.g., milliliters of blood). Several statistical reasons (resolving very small differences require large sample numbers) as well as traditional reasons (limited marker availability for FISH) have cemented this practice. The instant application shows how NIPT by cfDNA analysis is possible from ultra-low input amounts. See Examples 1-5. Methods, devices, systems and kits disclosed herein combine existing methods for high efficiency library creation, with low level DNA amplification (e.g., 8-10 cycles) in a novel way to enable NIPT from minimal sample volumes.

The devices and methods disclosed herein eliminate the need for a venipuncture, thereby providing for NIPT at point of care with a significant reduction in cost of testing. Since the fetal fraction in maternal blood can be low and maternal cell free nucleic acids can vary, it was unexpected that the methods, systems and devices disclosed herein would successfully reveal reliable and useful genetic information about a fetus. Maternal biology is always changing and there is a lot of variability in maternal cell-free nucleic acids of maternal subjects. There are cell-free nucleic acids from various organs of the mother (e.g., liver, skin) that contribute to circulating cell-free nucleic acids and the biology of those organs can change with age, disease, infection, and even time of day. It was unpredictable that maternal representation is reproducible enough to compare cell-free fetal nucleic acids from a test subject to cell-free fetal nucleic acids from a reference/control subject. One has to experimentally prove that the host background DNA is actually giving a stable enough distribution so that a trisomy or other genomic variations can be accurately detected.

Disclosed herein are devices and methods for obtaining genetic information of a fetus. Devices and methods disclosed herein may be advantageously capable of obtaining genetic information at very early stages of gestation. Devices and methods disclosed herein may obtain genetic information of a fetus in the privacy of a home, without the need for laboratory equipment and without the risk of sample swapping. Genetic information can be detected in minutes or seconds with devices, systems, kits and methods disclosed herein.

Disclosed herein are devices and methods for analyzing cell-free fetal nucleic acids from a biological fluid sample of a pregnant subject.

In some aspects, the devices and methods disclosed herein are useful for analyzing cell-free nucleic acids from a fetus, referred to herein as “cell free fetal nucleic acids.” In some instances, cell-free fetal nucleic acids are from at least one cell of the fetus, at least one cell of the placenta, or a combination thereof. Prenatal applications of cell-free fetal nucleic acids in maternal blood are presented with the additional challenge of analyzing cell-free fetal nucleic acids in the presence of cell-free maternal nucleic acids, the latter of which create a large background signal to the former. For example, a sample of maternal blood may contain about 500 to 2000 genome equivalents of total cell free DNA (maternal and fetal) per milliliter of whole blood. The fetal fraction in blood sampled from pregnant women may be around 10%, about 50 to 200 fetal genome equivalents per ml. Furthermore, the process of obtaining cell-free nucleic acids may involve obtaining plasma or serum from the blood. If not performed carefully, blood cells may be destroyed, releasing additional cellular nucleic acids into the sample, creating additional background signal to the fetal cell-free nucleic acids. The typical white cell count is around 4*10\hat{ }6 to 10*10\hat{ }6 cells per ml of blood and therefore the available nuclear DNA is around 10,000 times higher than the overall cell-free DNA (cfDNA). Consequently, even if only a small fraction of maternal white blood cells is destroyed, releasing nuclear DNA into the plasma or serum, the fetal fraction is reduced dramatically. For example, a white cell degradation of 0.01% may reduce the fetal fraction from 10% to about 5%. Devices and methods disclosed herein aim to reduce these background signals.

### Diseases and Conditions

Methods may comprise detecting the presence of a disease or condition based on the detecting. Methods may comprise detecting the risk of a disease or condition based on the detecting. Methods may comprise detecting the status of a disease or condition based on the detecting. Methods may comprise monitoring the status of a disease or condition based on the detecting. Methods may comprise administering a therapy based on the detecting. Methods may comprise modifying the dose of a drug that is being administered to the subject based on the detecting. Methods may comprise monitoring the response of a subject to a therapy based on the detecting. For example, the disease may be a cancer and the therapy may be a chemotherapy. Other cancer therapies include, but are not limited to antibodies, antibody-drug conjugates, antisense molecules, engineered T cells, and radiation. Methods may comprise further testing a subject based on the detecting. For example, the disease may be cancer and further testing may include, but is not limited to imaging (e.g., CAT-SCAN, PET-SCAN), and performing a biopsy.

Disclosed herein are devices and methods for detecting the presence, absence, or severity of a disease or condition in a subject. In some instances, the disease or condition is due to a genetic mutation. The genetic mutation may be inherited (e.g., the mutation was present in an ancestor or relative). The genetic mutation may be a spontaneous mutation (e.g., an error in DNA replication or repair). The genetic mutation may be due to exposure to an environmental factor (e.g., UV light, carcinogen). By way of non-limiting example, the genetic mutation may be selected from a frameshift mutation, an insertion mutation, a deletion mutation, a substitution mutation, a single nucleotide polymorphism, a copy number variation, and a chromosomal translocation.

In some instances, the disease or condition is due to an environmental factor (e.g., carcinogen, diet, stress, pathogen). In some instances, the environmental factor causes a genetic mutation. In other instances, the environmental factor does not cause a genetic mutation. In some instances, the environmental factor causes a change in one or more epigenetic modifications in a subject relative to a healthy individual. In some instances, the environmental factor causes a change in one or more epigenetic modifications in a subject relative to that of the subject at an earlier time point.

Devices and methods disclosed herein may be used to detect or monitor a disease or condition that affects one or more tissues, organs or cell types. The disease or condition may cause a release of nucleic acids from one or more tissues, organs or cell types. The disease or condition may increase a release of nucleic acids from one or more tissues, organs or cell types relative to a corresponding release occurring in a healthy individual. A tissue may be classified as epithelial, connective, muscle, or nervous tissue. Non-limiting examples of tissues are adipose, muscle, connective tissue, mammary tissue, and bone marrow. Non-limiting examples of organs are brain, thymus, thyroid, lung, heart, spleen, liver, kidney, pancreas, stomach, small intestine, large intestine, colon, prostate, ovary, uterus, and urinary bladder. Non-limiting examples of cell types are endothelial cells, vascular smooth muscle cells, cardiomyocytes, hepatocytes, pancreatic beta cells, adipocytes, neurons, endometrial cells, immune cells (T cells, B cells, dendritic cells, monocytes, macrophages, Kupffer cells, microglia).

Devices and methods disclosed herein may be used to detect or monitor general health. Devices and methods disclosed herein may be used to detect or monitor fitness. Devices and methods disclosed herein may be used to detect or monitor the health of an organ transplant recipient and/or the health of the transplanted organ.

The disease or condition may comprise an abnormal cell growth or proliferation. The disease or condition may comprise leukemia. Non-limiting types of leukemia include acute lymphoblastic leukemia (ALL), chronic lymphocytic leukemia (CLL), acute myelogenous leukemia (AML), chronic myelogenous leukemia (CML), and hairy cell leukemia (HCL). The disease or condition may comprise a lymphoma. The lymphoma may be a non-Hodgkin's lymphoma (e.g., B cell lymphoma, diffuse large B-cell lymphoma, T cell lymphoma, Waldenstrom macroglobulinemia) or a Hodgkin's lymphoma. The disease or condition may comprise a cancer. The cancer may be breast cancer. The cancer may be lung cancer. The cancer may be esophageal cancer. The cancer may be pancreatic cancer. The cancer may be ovarian cancer. The cancer may be uterine cancer. The cancer may be cervical cancer. The cancer may be testicular cancer. The cancer may be prostate cancer. The cancer may be bladder cancer. The cancer may be colon cancer. The cancer may be a sarcoma. The cancer may be an adenocarcinoma. The cancer may be isolated, that is it has not spread to other tissues besides the organ or tissue where the cancer originated. The cancer may be metastatic. The cancer may have spread to neighboring tissues. The cancer may have spread to cells, tissues or organs in physical contact with the organ or tissue where the cancer originated. The cancer may have spread to cells, tissues or organs not in physical contact with the organ or tissue where the cancer originated. The cancer may be in an early stage, such as Stage 0 (abnormal cell with the potential to become cancer) or Stage 1 (small and confined to one tissue). The cancer may be intermediate, such as Stage 2 or Stage 3, grown into tissues and lymph nodes in physical contact with the tissue of the original tumor. The cancer may be advanced, such as Stage 4 or Stage 5, wherein the cancer has metastasized to tissues that are distant (e.g., not adjacent or in physical contact) to the tissue of the original tumor. In some instances, the cancer is not advanced. In some instances, the cancer is not metastatic. In some instances, the cancer is metastatic.

The disease or condition may comprise a metabolic disorder. Metabolic conditions and diseases, include, but are not limited to obesity, a thyroid disorder, hypertension, type 1 diabetes, type 2 diabetes, non-alcoholic steatohepatitis, coronary artery disease, and atherosclerosis.

The disease or condition may comprise a cardiovascular condition. Non-limiting examples of cardiovascular conditions are atherosclerosis, myocardial infarction, pericarditis, myocarditis, ischemic stroke, hypertensive heart disease, rheumatic heart disease, cardiomyopathy, congenital heart disease, valvular heart disease, carditis, aortic aneurysms, peripheral artery disease, thromboembolic disease, and venous thrombosis.

The disease or condition may comprise a neurological disorder. The neurological disorder may comprise a neurodegenerative disease. Non-limiting examples of neurodegenerative and neurological disorders are Alzheimer's disease, Parkinson's disease, Huntington's disease, Spinocerebellar ataxia, amyotrophic lateral sclerosis (ALS), motor neuron disease, chronic pain, and spinal muscular atrophy. Devices, systems, kits and methods disclosed herein may be used to test for, detect, and/or monitor a psychiatric disorder in a subject and/or a response to a drug to treat the psychiatric disorder.

The disease or condition may comprise an infection. The disease or condition may be caused by an infection. The disease or condition may be exacerbated by an infection. The infection may be a viral infection. The infection may be a bacterial infection. The infection may be a fungal infection.

The disease or condition may be associated with aging. Disease and conditions associated with aging include, but are not limited to, cancer, osteoporosis, dementia, macular degeneration, metabolic conditions, and neurodegenerative disorders.

The disease or condition may be a blood disorder. Non-limiting examples of blood disorders are anemia, hemophilia, blood clotting and thrombophilia. For example, detecting thrombophilia may comprise detecting a polymorphism present in a gene selected from Factor V Leiden (FVL), prothrombin gene (PT G20210A), and methylenetetrahydrofolate reductase (MTHFR).

The disease or condition may be an allergy or intolerance to a food, liquid or drug. By way of non-limiting example, a subject can be allergic or intolerant to lactose, wheat, soy, dairy, caffeine, alcohol, nuts, shellfish, and eggs. A subject could also be allergic or intolerant to a drug, a supplement or a cosmetic. In some instances, methods comprise analyzing genetic markers that are predictive of skin type or skin health.

In some instances, the condition is associated with an allergy. In some instances, the subject is not diagnosed with a disease or condition, but is experiencing symptoms that indicate a disease or condition is present. In other instances, the subject is already diagnosed with a disease or condition, and the devices, systems, kits and methods disclosed herein are useful for monitoring the disease or condition, or an effect of a drug on the disease or condition.

### Chromosomal Abnormalities

Disclosed herein are devices and methods for detecting chromosomal abnormalities. Those of skill in the field may also refer to chromosomal abnormalities as chromosomal aberrations. In some instances, the chromosomal abnormality is a chromosomal duplication. In some instances, the chromosomal abnormality is a chromosomal deletion. In some instances, the chromosomal abnormality is deletion of an arm of a chromosome. In some instances, the chromosomal abnormality is a partial deletion of an arm of a chromosome. In some instances, the chromosomal abnormality comprises at least one copy of a gene. In some instances, the chromosomal abnormality is due to a breakage of a chromosome. In some instances, the chromosomal abnormality is due to a translocation of a portion of a first chromosome to a portion of a second chromosome.

Many known chromosomal abnormalities results in chromosomal disorders. Thus, the devices, systems, kits and methods disclosed herein may be used for detecting chromosomal disorders. By way of non-limiting example, chromosomal disorders include Down's syndrome (trisomy 21), Edward's syndrome (trisomy 18), Patau syndrome (trisomy 13), Cri du chat syndrome (partial deletion of short arm of chromosome 5), Wolf-Hirschhorn syndrome (deletion of short arm of chromosome 4), Jacobsen syndrome (deletion of long arm of chromosome 11), diGeorge's syndrome (small deletion of chromosome 22), Klinefelter's syndrome (presence of additional X chromosome in males), and Turner syndrome (presence of only a single X chromosome in females).

### Subjects

Disclosed herein are devices and methods for analyzing a biological component in a sample from a subject. The subject may be human. The subject may be non-human. The subject may be non-mammalian (e.g., bird, reptile, insect). In some instances, the subject is a mammal. In some instances, the mammal is female. In some instances, the subject is a human subject. In some instances, the mammal is a primate (e.g., human, great ape, lesser ape, monkey). In some instances, the mammal is canine (e.g., dog, fox, wolf). In some instances, the mammal is feline (e.g., domestic cat, big cat). In some instances, the mammal is equine (e.g., horse). In some instances, the mammal is bovine (e.g., cow, buffalo, bison). In some instances, the mammal is a sheep. In some instances, the mammal is a goat). In some instances, the mammal is a pig. In some instances, the mammal is a rodent (e.g., mouse, rat, rabbit, guinea pig).

In some instances, a subject described herein is affected by a disease or a condition. Devices and methods disclosed herein may be used to test for the disease or condition, detect the disease or condition, and/or monitor the disease or condition. Devices, systems, kits and methods disclosed herein may be used to test for the presence of inherited traits, monitor fitness, and detect family ties.

Devices and methods disclosed herein may be used to test for, detect, and/or monitor cancer in a subject. Non-limiting examples of cancers include breast cancer, prostate cancer, skin cancer, lung cancer, colorectal cancer/colon cancer, bladder cancer, pancreatic cancer, lymphoma, and leukemia.

In some instances, the condition is associated with an allergy. In some instances, the subject is not diagnosed with a disease or condition, but is experiencing symptoms that indicate a disease or condition is present. In other instances, the subject is already diagnosed with a disease or condition, and the devices, systems, kits and methods disclosed herein are useful for monitoring the disease or condition, or an effect of a drug on the disease or condition.

Disclosed herein are devices and methods for analyzing cell-free nucleic acids from a fetus in a maternal biological sample from a pregnant subject. Generally, the pregnant subject is a human pregnant subject. However, one of skill in the art would understand that the instant disclosure could be applied to other mammals, perhaps for breeding purposes on farms or in zoos. In some instances, the pregnant subject is euploid. In some instances, the pregnant subject comprises an aneuploidy. In some instances, the pregnant subject has a copy variation of a gene or portion thereof. In some instances, the pregnant subject has a genetic insertion mutation. In some instances, the pregnant subject has a genetic deletion mutation. In some instances, the pregnant subject has a genetic missense mutation. In some instances, the pregnant subject has a single nucleotide polymorphism. In some instances, the pregnant subject has a single nucleotide polymorphism. In some instances, the pregnant subject has translocation mutation resulting in a fusion gene. By way of non-limiting example, the BCR-ABL gene is a fusion gene that can be found on chromosome 22 of many leukemia patients. The altered chromosome 22 is referred to as the Philadelphia chromosome.

In some instances, the pregnant subject is about 2 weeks pregnant to about 42 weeks pregnant. In some instances, the pregnant subject is about 3 weeks pregnant to about 42 weeks pregnant. In some instances, the pregnant subject is about 4 weeks pregnant to about 42 weeks pregnant. In some instances, the pregnant subject is about 5 weeks pregnant to about 42 weeks pregnant. In some instances, the pregnant subject is about 6 weeks pregnant to about 42 weeks pregnant. In some instances, the pregnant subject is about 7 weeks pregnant to about 42 weeks pregnant. In some instances, the pregnant subject is about 8 weeks pregnant to about 42 weeks pregnant.

In some instances, the pregnant subject is at fewer than about 6 weeks, about 7 weeks, about 8 weeks, about 9 weeks, about 10 weeks, about 12 weeks, about 16 weeks, about 20 weeks, about 21 weeks, about 22 weeks, about 24 weeks, about 26 weeks, or about 28 weeks of gestation. In some instances, the pregnant subject is as few as 5 weeks pregnant. In some instances, the human subject is a pregnant human female who has reached at least about 5 weeks, at least about 6 weeks, at least about 7 weeks, or at least about 8 weeks of gestation. In some instances, the human subject is a pregnant human female who has reached at least about 5 to about 8 weeks of gestation. In some instances, the human subject is a pregnant human female who has reached at least about 5 to about 8, at least about 5 to about 12, at least about 5 to about 16, at least about 5 to about 20, at least about 6 to about 21, at least about 6 to about 22, at least about 6 to about 24, at least about 6 to about 26, at least about 6 to about 28, at least about 6 to about 9, at least about 6 to about 12, at least about 6 to about 16, at least about 6 to about 20, at least about 6 to about 21, at least about 6 to about 22, at least about 6 to about 24, at least about 6 to about 26, or at least about 6 to about 28 weeks of gestation. In some instances, the human subject is a pregnant human female who has reached at least about 7 to about 8, at least about 7 to about 12, at least about 7 to about 16, at least about 7 to about 20, at least about 7 to about 21, at least about 7 to about 22, at least about 7 to about 24, at least about 7 to about 26, at least about 7 to about 28, at least about 8 to about 9, at least about 8 to about 12, at least about 6 to about 16, at least about 8 to about 20, at least about 8 to about 21, at least about 6 to about 22, at least about 8 to about 24, at least about 8 to about 26, or at least about 8 to about 28 weeks of gestation. In some instances, gestation times are detected by measuring the gestation time from the first day of the last menstrual period.

In some instances, the biological sample is a maternal body fluid sample obtained from a pregnant subject, a subject suspected of being pregnant, or a subject that has given birth recently, e.g., within the past day. In some instances, the subject is a mammal. In some instances, the mammal is female. In some instances, the mammal is a primate (e.g., human, great ape, lesser ape, monkey), canine (e.g., dog, fox, wolf), feline (e.g., domestic cat, big cat), equine (e.g., horse), bovine (e.g., cow, buffalo, bison), ovine (e.g., sheep), caprine (e.g., goat) porcine (e.g., pig), a rhinoceros, or a rodent (e.g., mouse, rat, rabbit, guinea pig). In some instances, the subject is a pregnant human female in her first, second, or third trimester of pregnancy. In some instances, the human subject is a pregnant human female at fewer than about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, or about 40 weeks gestation.

### NUMBERED EMBODIMENTS

The following embodiments recite nonlimiting permutations of combinations of features disclosed herein. Other permutations of combinations of features are also contemplated. In particular, each of these numbered embodiments is contemplated as depending from or relating to every previous or subsequent numbered embodiment, independent of their order as listed.

Embodiment 1: A method comprising: (a) providing or obtaining a blood sample obtained from an individual; (b) applying a positive pressure to a starting volume of the blood sample such that the blood sample is pushed or forced into a filter or a membrane, wherein the starting volume of the blood sample is not more than about 1 milliliter (mL); (c) filtering the blood sample through the filter or the membrane to separate plasma from the blood sample, and (d) collecting the plasma in a collection vessel, wherein a volume of the plasma is greater than about 25% of the starting volume of the blood sample.

Embodiment 2: The method of embodiment 1, wherein the volume of the plasma is greater than about 30%, greater than about 35%, or greater than about 40% of the starting volume of the blood sample.

Embodiment 3: The method of embodiment 1 or 2, wherein at least about 60%, at least about 65%, at least about 70%, at least about 75%, at least about 80%, at least about 85%, at least about 90%, or greater, of the total amount of plasma present in the blood sample is collected.

Embodiment 4: The method of any one of embodiments 1-3, wherein the plasma comprises cell-free nucleic acids.

Embodiment 5: The method of embodiment 4, wherein the method results in an enrichment of cell-free nucleic acids.

Embodiment 6: The method of any one of embodiments 1-5, wherein the starting volume of the blood sample is not more than about 500 microliters (μL), not more than about 250 μL, not more than about 150 μL, not more than about 100 μL, not more than about 80 μL, not more than about 60 μL, not more than about 50 μL, not more than about 40 μL, or not more than about 25 μL.

Embodiment 7: The method of any one of embodiments 1-6, wherein cellular nucleic acids are reduced in the plasma.

Embodiment 8: The method of any one of embodiments 1-7, wherein the plasma is substantially free of cellular nucleic acids.

Embodiment 9: The method of any one of embodiments 1-8, wherein cells, cell fragments, microvesicles, or any combination thereof, are reduced in the plasma.

Embodiment 10: The method of any one of embodiments 1-9, wherein the blood sample is or comprises capillary blood.

Embodiment 11: The method of any one of embodiments 1-10, wherein the blood sample is or comprises whole blood or one or more blood components.

Embodiment 12: The method of any one of embodiments 1-11, wherein a volume of plasma collected in (d) is greater than a volume of plasma collected using an equivalent method using negative pressure or vacuum.

Embodiment 13: The method of any one of embodiments 1-12, wherein a volume of plasma collected in (d) is at least about 10%, at least about 25%, at least about 50%, at least about 75%, at least about 100%, or greater than a volume of plasma collected using an equivalent method using negative pressure or vacuum.

Embodiment 14: The method of any one of embodiments 1-13, wherein the positive pressure is of an amount of less than about 4 psi, an amount of 4 psi to about 11 psi, or an amount greater than about 11 psi.

Embodiment 15: The method of any one of embodiments 1-14, wherein the method is performed in 1 minute or less.

Embodiment 16: The method of any one of embodiments 1-15, wherein the method does not result in substantial lysis or disruption of white blood cells.

Embodiment 17: The method of any one of embodiments 1-16, wherein the positive pressure is selected such that hemolysis of red blood cells may occur, but white blood cells are not lysed or disrupted.

Embodiment 18: The method of any one of embodiments 1-17, wherein the cell-free nucleic acids are deoxyribonucleic acid.

Embodiment 19: The method of any one of embodiments 1-18, wherein the cell-free nucleic acids comprise cell-free nucleic acids from a tumor.

Embodiment 20: The method of any one of embodiments 1-18, wherein the cell-free nucleic acids comprise cell-free nucleic acids from a fetus.

Embodiment 21: The method of any one of embodiments 1-18, wherein the cell-free nucleic acids comprise cell-free nucleic acids from a transplanted tissue or organ.

Embodiment 22: The method of any one of embodiments 1-21, wherein the cell-free nucleic acids comprise from about 10⁴ to about 10⁹ cell-free nucleic acid molecules.

Embodiment 23: The method of any one of embodiments 1-22, wherein the blood sample is obtained from the individual by a finger prick.

Embodiment 24: The method of any one of embodiments 1-23, wherein the method is performed on a point-of-care or point-of-need device.

Embodiment 25: A device comprising: (a) a positive pressure source configured to exert a positive pressure on a blood sample and to push or force portions of the blood sample into a filter or membrane; and (b) the filter or membrane configured to separate plasma from the blood sample, wherein the device is configured to separate a volume of plasma from the blood sample that is greater than about 25% of an input volume of the blood sample.

Embodiment 26: The device of embodiment 25, wherein the filter or membrane is configured to separate plasma from an input volume of no more than about 1 milliliter (mL) of the blood sample.

Embodiment 27: The device of embodiment 25, wherein the filter or membrane is configured to separate plasma from an input volume of no more than about 500 microliters (μL) of the blood sample.

Embodiment 28: The device of embodiment 25, wherein the filter or membrane is configured to separate plasma from an input volume of no more than about 100 microliters (μL) of the blood sample.

Embodiment 29: The device of embodiment 25, wherein the filter or membrane is configured to separate plasma from an input volume of no more than about 50 microliters (μL) of the blood sample.

Embodiment 30: The device of any one of embodiments 25-29, wherein the volume of the plasma is greater than about 30%, greater than about 35%, or greater than about 40% of the starting volume of the blood sample.

Embodiment 31: The device of any one of embodiments 25-30, wherein at least about 60%, at least about 65%, at least about 70%, at least about 75%, at least about 80%, at least about 85%, at least about 90%, or greater, of the total amount of plasma present in the blood sample is collected.

Embodiment 32: The device of any one of embodiments 25-31, wherein the positive pressure source is a mechanical positive pressure source.

Embodiment 33: The device of any one of embodiments 25-32, wherein the positive pressure source comprises a material that possesses a spring force.

Embodiment 34: The device of any one of embodiments 25-33, wherein the positive pressure source comprises a foam material.

Embodiment 35: The device of any one of embodiments 25-34, further comprising a structure configured to collect plasma from the filter or membrane and transport the plasma to a collection vessel, a collection region, or a collection structure within the device.

Embodiment 36: The device of any one of embodiments 25-35, wherein the device is a point-of-care or point-of-need device.

Embodiment 37: The device of any one of embodiments 25-36, wherein the filter or membrane is configured to remove or reduce a population of cells, cell fragments, microvesicles, or any combination thereof from the blood sample.

Embodiment 38: The device of any one of embodiments 25-37, further comprising a sample inlet for introducing the blood sample into the device.

Embodiment 39: The device of any one of embodiments 25-38, wherein the sample inlet is configured to be sealed prior to exertion of the positive pressure on the blood sample.

Embodiment 40: The device of any one of embodiments 25-39, wherein the sample inlet is configured to be open during exertion of the positive pressure on the blood sample.

Embodiment 41: The device of any one of embodiments 25-40, wherein the filter or membrane comprises a plurality of pores.

Embodiment 42: The device of embodiment 41, wherein a plurality of pores located at a first side of the filter or membrane have an average pore size that is greater than an average pore size of a plurality of pores located at a second side of the filter or membrane.

Embodiment 43: The device of any one of embodiments 25-42, wherein the device comprises two or more filters or membranes, each comprising a plurality of pores with an average pore size that is different for each filter or membrane.

Embodiment 44: A method comprising: (a) providing or obtaining a blood sample obtained from an individual; (b) centrifuging the blood sample such that portions of the blood sample are forced or pushed through a filter or a membrane; (c) filtering the blood sample through the filter or the membrane to separate plasma from the blood sample, and (d) collecting the plasma in a collection vessel, wherein a volume of the plasma is greater than about 25% of an input volume of the blood sample.

Embodiment 45: The method of embodiment 44, wherein the volume of the plasma is greater than about 30%, greater than about 35%, or greater than about 40% of the input volume of the blood sample.

Embodiment 46: The method of embodiment 44 or 45, wherein the plasma comprises cell-free nucleic acids.

Embodiment 47: The method of embodiment 46, wherein the method results in an enrichment of cell-free nucleic acids.

Embodiment 48: The method of any one of embodiments 44-47, wherein the input volume of the blood sample is not more than about 500 microliters (μL), not more than about 250 μL, not more than about 150 μL, not more than about 100 μL, not more than about 80 μL, not more than about 60 μL, not more than about 50 μL, not more than about 40 μL, or not more than about 25 μL.

Embodiment 49: The method of any one of embodiments 44-48, wherein cellular nucleic acids are reduced in the plasma.

Embodiment 50: The method of any one of embodiments 44-49, wherein the plasma is substantially free of cellular nucleic acids.

Embodiment 51: The method of any one of embodiments 44-50, wherein cells, cell fragments, microvesicles, or any combination thereof, are reduced in the plasma.

Embodiment 52: The method of any one of embodiments 44-51, wherein the blood sample is or comprises capillary blood.

Embodiment 53: The method of any one of embodiments 44-52, wherein the blood sample is or comprises whole blood or one or more blood components.

Embodiment 54: The method of any one of embodiments 44-53, wherein a volume of plasma collected in (d) is greater than a volume of plasma collected using an equivalent method using a centrifuge without the use of the filter or membrane.

Embodiment 55: The method of any one of embodiments 44-54, wherein a volume of plasma collected in (d) is at least about 10%, at least about 25%, at least about 50%, at least about 75%, at least about 100%, or greater than a volume of plasma collected using an equivalent method using a centrifuge without the use of the filter or membrane.

Embodiment 56: The method of any one of embodiments 44-55, wherein the method does not result in substantial lysis or disruption of white blood cells.

Embodiment 57: The method of any one of embodiments 44-56, wherein the cell-free nucleic acids are deoxyribonucleic acid.

Embodiment 58: The method of any one of embodiments 44-57, wherein the cell-free nucleic acids comprise cell-free nucleic acids from a tumor.

Embodiment 59: The method of any one of embodiments 44-58, wherein the cell-free nucleic acids comprise cell-free nucleic acids from a fetus.

Embodiment 60: The method of any one of embodiments 44-59, wherein the cell-free nucleic acids comprise cell-free nucleic acids from a transplanted tissue or organ.

Embodiment 61: The method of any one of embodiments 44-60, wherein the cell-free nucleic acids comprise from about 10⁴ to about 10⁹ cell-free nucleic acid molecules.

Embodiment 62: The method of any one of embodiments 44-61, wherein the whole blood sample is obtained from the individual by a finger prick.

Embodiment 63: The method of any one of embodiments 44-62, wherein the method is performed in a laboratory setting.

Embodiment 64: A device comprising: (a) a sample inlet configured to introduce a blood sample into the device; (b) a filter or membrane configured to separate plasma from the blood sample; (c) a collection vessel configured to collect the plasma; and (d) an adapter, removably or permanently affixed to the device, configured to attach the device to a centrifuge.

Embodiment 65: The device of embodiment 64, wherein the adapter is configured to attach the device to a centrifuge tube, to a plate, or interfaces to the centrifuge without an additional centrifuge tube.

Embodiment 66: The device of embodiment 64 or 65, wherein the filter or membrane is configured to separate plasma from an input volume of no more than about 1 milliliter (mL) of the blood sample.

Embodiment 67: The device of any one of embodiments 64-66, wherein the filter or membrane is configured to separate plasma from an input volume of no more than about 500 microliters (μL) of the blood sample.

Embodiment 68: The device of any one of embodiments 64-67, wherein the filter or membrane is configured to separate plasma from an input volume of no more than about 100 microliters (μL) of the blood sample.

Embodiment 69: The device of any one of embodiments 64-68, wherein the filter or membrane is configured to separate plasma from an input volume of no more than about 50 microliters (μL) of the blood sample.

Embodiment 70: The device of any one of embodiments 64-69, wherein the filter or membrane is configured to separate plasma from an input volume of no more than about 25 microliters (μL) of the blood sample.

Embodiment 71: The device of any one of embodiments 64-70, wherein the filter or membrane is configured to remove or reduce a population of cells, cell fragments, microvesicles, or any combination thereof from the blood sample.

Embodiment 72: The device of any one of embodiments 64-71, wherein the filter or membrane comprises a plurality of pores.

Embodiment 73: The device of embodiment 72, wherein a plurality of pores located at a first side of the filter or membrane have an average pore size that is greater than an average pore size of a plurality of pores located at a second side of the filter or membrane.

Embodiment 74: The device of any one of embodiments 64-73, wherein the device comprises two or more filters or membranes, each comprising a plurality of pores with an average pore size that is different for each filter or membrane.

### EXAMPLES

The following examples are given for the purpose of illustrating various embodiments of the devices, systems and kits disclosed herein and are not meant to limit the present devices, systems and kits in any fashion. The present examples, along with the methods described herein are presently representative of preferred embodiments, are exemplary, and are not intended as limitations on the scope of the invention. Changes therein and other uses which are encompassed within the spirit of the devices, systems and kits disclosed herein as defined by the scope of the claims will occur to those skilled in the art.

### Example 1: Capillary Blood-Collection and Characterization

The collection of capillary blood by finger prick offers a convenient and distributable methodology for the acquisition of patient blood and blood products for biological testing at-home, point-of-care or at a clinical laboratory via sample shipping. As with venous collected blood, capillary blood contains circulating cell-free DNA (ccfDNA) that can be used as a biomarker for genetic testing. However, ccfDNA extracted from capillary blood has not been well-characterized to determine if any differences exist between blood collected from a finger prick vs. that collected by venipuncture.

The goal of the experiment described in this example was to study multiple parameters involved in the process of capillary blood collection via finger prick and its effects on ccfDNA as an analyte for non-invasive prenatal testing (NIPT). In order to evaluate different parameters, ccfDNA yield, size, and genome representation of ccfDNA and correlation of capillary vs. venous-collected blood were measured via quantitative real-time polymerase chain reaction (qPCR) and SBS.

The following is a brief outline of the methods of the aforementioned experimental study. Capillary and venous blood were collected under an approved IRB protocol, where applicable. Each collection had a minimum of 50 μL capillary blood collected using a capillary blood collection protocol or variations thereof depending on the parameters being studied. Venous blood was collected using a venous blood collection protocol.

Plasma separation was performed via centrifugation. The time from blood collection to plasma separation varied depending on the study parameters. The majority of samples were processed within 4 hours of blood draw.

Hemoglobin levels were measured at 414 nm on a nanodrop from 1-2 μL of plasma. A serial dilution of hemolyzed blood mixed with plasma was established to estimate the level of hemolysis per sample.

Extraction of ccfDNA was conducted based on an input of 10 μL of maternal plasma and an elution volume of 20 μL or less.

Library preparation was conducted using a kit. Library QC for size and relative concentration was conducted using a Bioanalyzer 2100 with a high-sensitivity DNA chip. Libraries were quantified for dilution and pool using a Qubit 2.0 fluorometer.

Standard curve qPCR was conducted using a QuantStudio3 instrument and Taqman biochemistry. Assays specific for a multicopy region of the Y-chromosome and a total copy assay were used for sample quantification.

Sequencing-by-synthesis was conducted on an Illumina NextSeq 550 with a mid-output reagent kit. Sample sheets were uploaded to a Basespace account and Fastq files were generated and stored on Basespace. Following Fastq generation, the alignment and classification were uploaded to a processing pipeline on AWS.

Table 1 summarizes the capillary blood collection numbers to date. In total, 1,565 samples derived from finger prick have been conducted for the study of multiple parameters surrounding collection and processing methodologies. All capillary collections thus far have obtained more than the required 50 μl of blood via finger prick (yields of 200 μl or more are obtained on average) and all have yielded well above the 10 μl of maternal plasma required for ccfDNA extraction (yields of 80 μl or more are obtained on average). All samples to date have been successfully extracted using an ultra-low volume ccfDNA extraction methodology with analysis via qPCR or sequencing-by-synthesis using an ultra-low input workflow starting with 10 μl of maternal plasma.

TABLE 1
Total number of capillary blood derived samples collected via finger prick.

| Parameter | Value |
|---|---|
| Total Capillary Samples | N = 1565 |
| Minimum Blood Volume Collected (μL) | >50 |
| Minimum Plasma Yield (μL) | >20 |

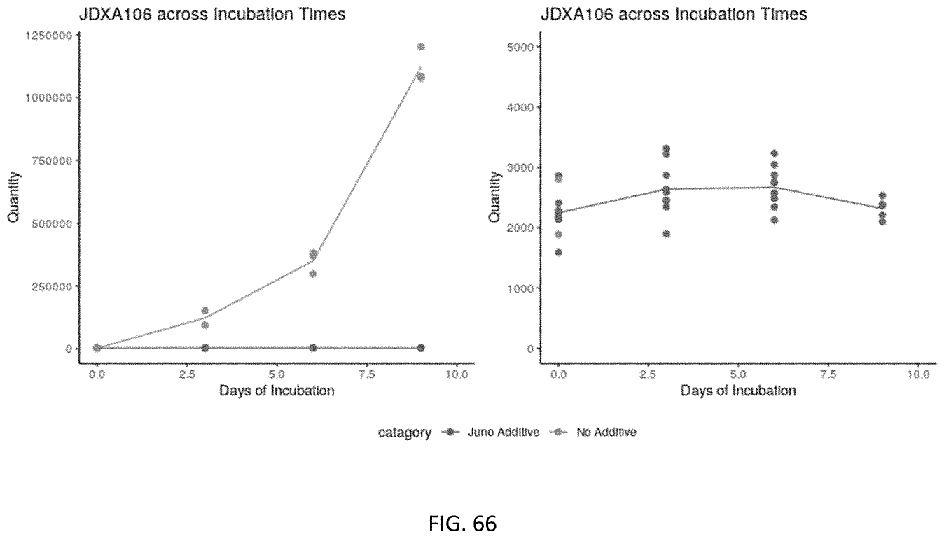

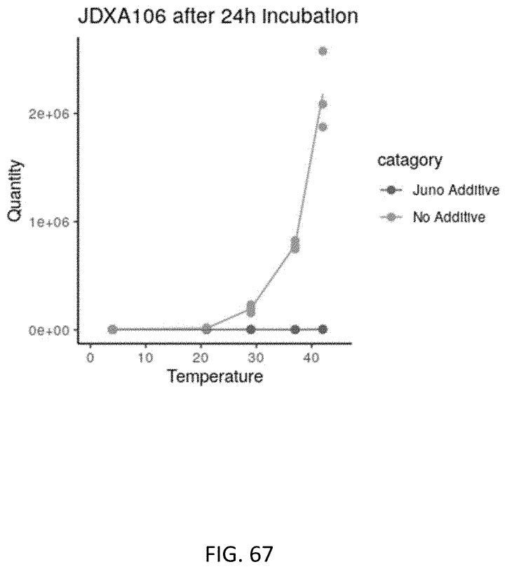

As is known with venous collected blood, duration of time and temperature at which EDTA blood is stored influence the degree of leukocyte degradation and subsequent release of cellular DNA into the blood sample. This same effect was confirmed to be true with capillary blood collected into EDTA microtubes. It was found that cellular DNA levels begin increasing after 6+ hours storage at room temperature while ccfDNA levels stay steady. This effect continued for up to 5 days and can be increased by incubating the blood at 37 degrees Celsius or slowed by storage of the blood at 4 degrees Celsius. Processing the capillary blood to plasma within 4-6 hours eliminates the effect and the plasma was found to be stable up to 6 or more days at ambient temperature. Blood subjected to incubation at 50 degrees Celsius was found to be unusable after overnight incubation. Based on these findings, two paths to eliminate the introduction of cellular DNA into capillary collected blood were investigated. The first approach used a cellular stabilizing reagent which can be introduced into the collection microtube during manufacturing. The initial data showed stabilization of white blood cells found in blood up to a minimum of 9 days and up to a temperature of 37 degrees Celsius (FIG. 66 and FIG. 67) which would allow for shipping of capillary blood suitable for ultra-low input NIPT. Specifically, FIG. 66 shows a time course of Y chromosome-specific target sequence amplification via standard curve qPCR with ccfDNA extracted from blood either with or without a blood stabilization additive. As seen in FIG. 66, blood without additive shows very large increases in target amplification within 24 hours of collection. This increase represents the lysis of white blood cells within the blood. The blood aliquots with additive show no increase in ccfDNA signal from time 0 to 9 days indicating the white cells are stabilized over that time period and the fetal fraction of the sample would be stable for NIPT. FIG. 67 shows temperature titration (4-42 degrees Celsius) of Y chromosome-specific target sequence amplification via standard curve qPCR with ccfDNA extracted from blood either with or without a blood stabilization additive incubated at various temperatures for 24 hours. As seen in FIG. 67, blood without additive shows very large increases in target amplification within 24 hours of collection. This increase represents the lysis of white blood cells within the blood. The blood aliquots with additive show no increase in ccfDNA signal from temperatures of 4-37 degrees Celsius indicating the white cells are stabilized over that time period and the fetal fraction of the sample would be stable for NIPT.

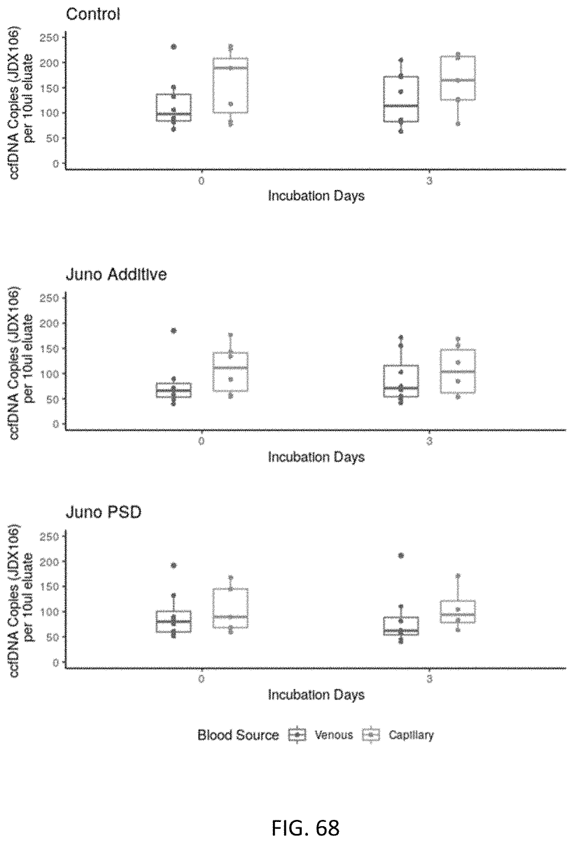

The second approach is a single use plasma separation device (PSD) which separates plasma from capillary blood at the time of collection, in preparation for shipping, eliminating the DNA containing cells from the sample with clinical sample studies ongoing. FIG. 68 shows data for paired venous and capillary blood samples collected from pregnant women carrying a male fetus at time 0 (2-4 hours after blood collection) and time 3 (72 hours or 3 days post blood collection stored at ambient temperature). The samples were collected into EDTA, EDTA+blood stabilization additive, or EDTA+PSD. All three methods show that more than adequate fetal ccfDNA copies are recovered at 0 and 3 days without loss of fetal DNA over time. For all methods, a higher amount of fetal ccfDNA was recovered from the capillary blood collection than the venous blood collection.

The studies presented herein for Example 1 found that capillary blood collected via finger prick is a robust matrix for the extraction of ccfDNA and downstream processes for analysis of nucleic acids such as qPCR and sequencing-by-synthesis. DNA yields and the ability to detect ccfDNA was equivalent to venous blood for qPCR and sequencing-by-synthesis based detection methodologies. Detectable levels of ccfDNA were found to be stable when capillary blood was incubated for 0-7 days at ambient or 37 degrees Celsius. The level of genomic DNA was found to increase over time indicating lysis of leukocytes and subsequent release of gDNA. Blood subjected to incubation at 50 degrees Celsius was found to be unusable after overnight incubation. Current studies using stabilizing reagents to reduce cellular degradation in blood show the desired effect as does direct plasma separation at the time of capillary blood collection. Both methods allow for shipping of capillary blood samples in a manner suitable for the ultra-low input NIPT process. Neither hemoglobin or hemolysis levels had a detectable effect on nucleic acid detection in capillary-collected blood. Size profiles and genome representation of capillary and venous ccfDNA were equivalent in the blood collected from pregnant women. Based on these studies it can be concluded that self-collected capillary blood is a safe and suitable matrix for genetic testing using ccfDNA as a template including ultra-low input NIPT.

### Example 2: Capillary Blood NIPT Clinical Performance

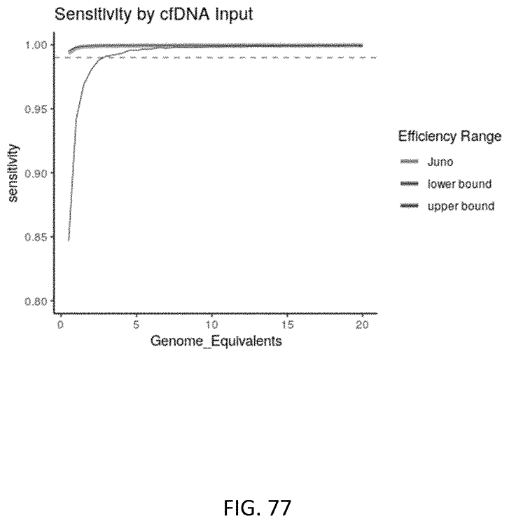

The collection of capillary blood by finger prick offers a convenient and distributable methodology for the acquisition of patient blood and blood products for biological testing at-home, at point-of-care or at a clinical laboratory via sample shipping. As with venous collected blood, capillary blood contains circulating cell-free DNA (ccfDNA) that can be used as a biomarker for genetic testing including non-invasive prenatal testing (NIPT). The current standard-of-care sample type for NIPT is venous blood which is then processed to plasma for analysis of ccfDNA from the mother and fetus. In Example 2, a study is described where the performance of capillary-collected blood from pregnant females is assessed as a viable source as it relates to a ultralow input NIPT assay from sample collection through aneuploidy classification. Previous ultralow input NIPT data using plasma derived from venous collected blood and simulations of sensitivity based on input genome equivalents (GE) (FIG. 77) suggests that capillary blood should perform well as tested in this study. Briefly, FIG. 77 simulations show that the minimum number of fetal ccfDNA copies or genome equivalents (GE) to achieve a sensitivity of 99% or better for detection of fetal trisomies against a maternal background is 1 GE or more given that conversion to whole genome library of a single fetal yields approximately 20,000,000 DNA fragments for sequencing. The accuracy of these simulations in this study on capillary collected blood were also analyzed and found to be accurate based on the data acquired and other data from larger studies using 10 μl of maternal plasma as the input. FIG. 77 shows the sensitivity for detecting fetal trisomies at different genome equivalent (GE) inputs assuming different process efficiencies for an ultralow input NIPT workflow. The dotted line represents a 99% sensitivity to detect a fetal trisomy. A low efficiency process (shown in grey) requires 4-5 GEs to achieve the 99% sensitivity level whereas a highly efficient process requires 1 or more GEs (dark grey). The process described herein tracks with the high efficiency simulations based on the sensitivity achieved to date in both capillary and venous collected samples. The simulations assume 4 million sequencing reads and a fetal fraction of 10%.

The following is a brief description of the aforementioned study and design. Capillary blood samples were collected from pregnant females at three collection sites under an approved TRB protocol. A total of 62 patients participated for the study. In this sample set 9 patients carried a fetus confirmed to have trisomy 21. Amongst the non-affected samples 3 had invasive testing results negative for Trisomy 21. The remaining 50 samples were presumed euploid samples from normal pregnancies. Twenty of those samples have had cell-free DNA testing (19 low risk, 1 no results at time of blood draw), 12 patients had only ultrasound screening (US) (12 normal results) and 4 patients had both NIPT and US (all 4 were low risk and normal scan); 14 patients had no form of prenatal screening at the time of collection.

Samples were collected under an approved IRB Protocol. Each patient had a minimum of 50 μL capillary blood collected using a capillary blood collection protocol. Capillary blood was collected in an EDTA microtube.

Plasma separation was performed via centrifugation. The time from blood collection to plasma separation varied slightly between collection sites. Samples collected at site A, were picked up and processed at the laboratory within 4 hours of blood draw. Samples collected at Site B were processed on site within 4 hours of blood collection. Only samples collected at Site C were shipped overnight to the laboratory and processed within 15 hours to 25 hours.

Extraction of ccfDNA was conducted based on an input of 10 μl of maternal plasma and an elution volume of 20 μl or less.

Standard curve qPCR was conducted using a QuantStudio3 instrument and Taqman biochemistry. Assays specific for a multicopy region of the Y-chromosome and a total copy assay were used for sample quantification.

Library preparation was conducted using a library preparation kit. Library QC for size and relative concentration was conducted using a Bioanalyzer 2100 with a high-sensitivity DNA chip. Libraries were quantified for dilution and pooling using a Qubit 2.0 Fluorometer.

SBS was conducted on an Illumina NextSeq 550 with a mid-output reagent kit. Sample sheets were uploaded to a Basespace account and Fastq files were generated and stored on Basespace. Following Fastq generation, the alignment and classification were uploaded on AWS. A pipeline was used in this study with a z-score cutoff of 3.5 for classification of positive vs. negative Trisomy 21 and a minimum of 2 million sequenced DNA fragments which equates to 4 million reads for classification.

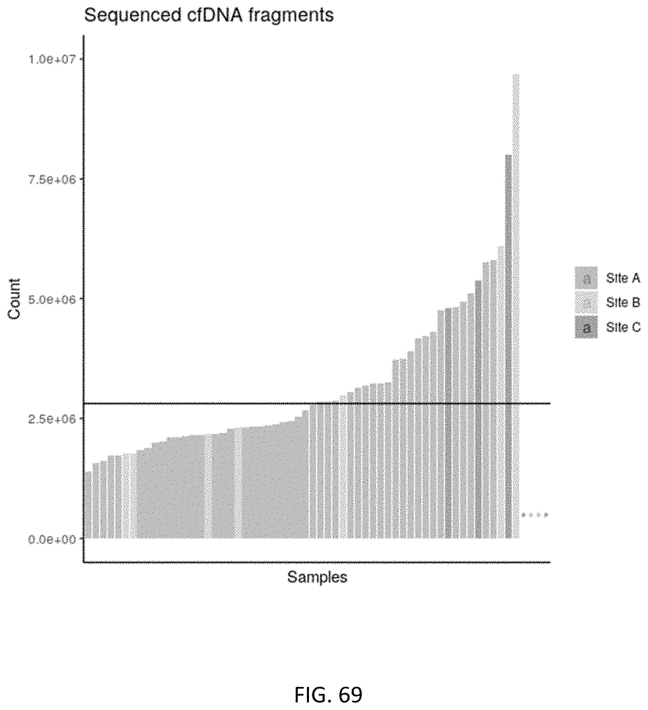

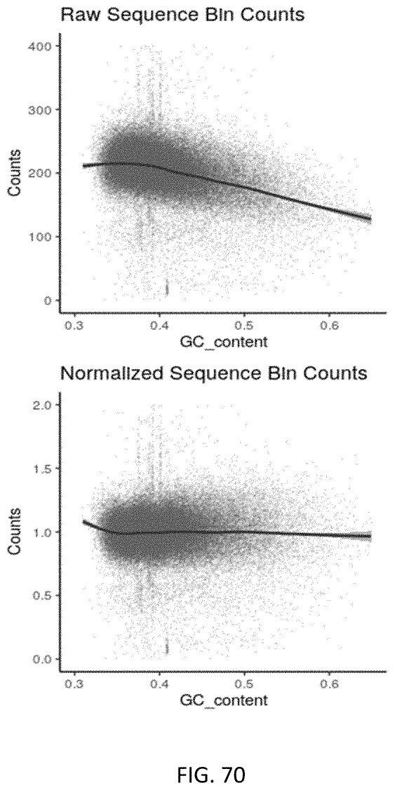

All capillary collections in the study obtained more than the required 50 μl of blood via finger prick and yielded well above the 10 μl of maternal plasma required for ccfDNA extraction. All samples were successfully sequenced based on the ultralow input NIPT workflow starting with 10 μl of maternal plasma. The number of sequenced DNA fragments ranged from 1.4M to 31M (FIG. 69). The median sequenced DNA fragments was 2.8M after alignment and QC, placing it in the acceptable range for T21 classification performance. Several samples had very high sequencing counts suggesting normalization for those samples can be improved. The GC-bias of the capillary samples looked as expected showing the well described GC sequencing bias (FIG. 70). For some samples, the bin representation counts are not only driven by statistical sampling and biological copy number variations but also GC content which has a major impact on the sequencing bias and the resulting bin count representation. The GC-bias therefore needs to be normalized or corrected to maximize classification performance. Here it was corrected using a simple loess regression, see FIG. 69. It is well known that in large datasets better normalization results can be achieved with more parameterized and better trained models. Loess regression was used in this study because of its simplicity and good performance in the dataset.

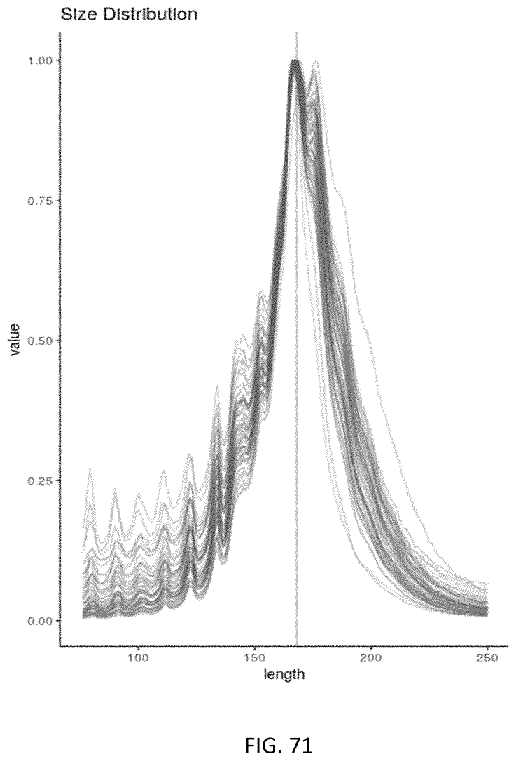

The size distribution for the capillary samples shows the expected maximum around 168 bp (FIG. 70). Some samples had low counts and showed greater variance in size distribution for both small and larger fragments, as seen in FIG. 71. No difference between euploid and trisomy samples was observed.

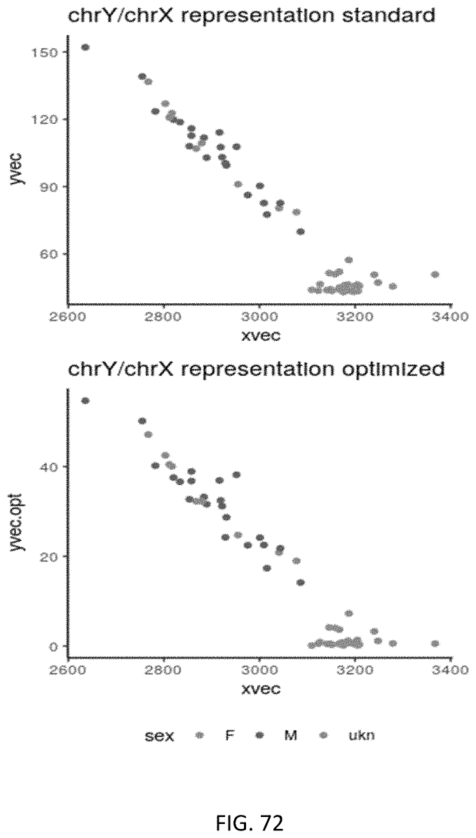

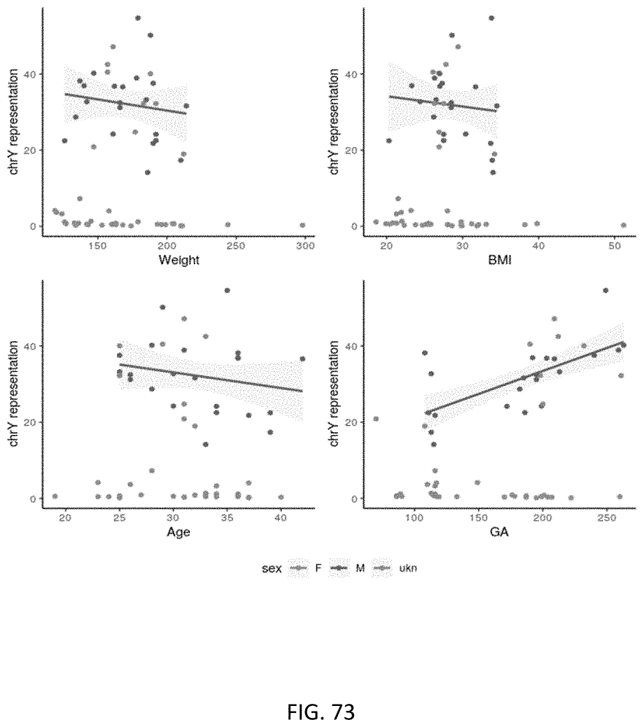

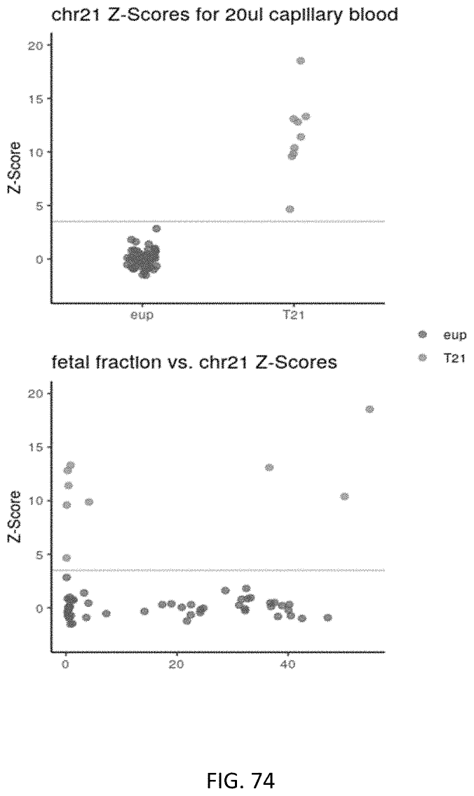

Fetal fraction was measured by the presence of Y chromosomal fragments in the study for male samples only. The representation for chromosome X and chromosome Y is calculated by adding all GC corrected and normalized and bins originating from the chromosome together. For samples with a male fetus a higher fetal fraction results in a higher chromosome Y representation with a simultaneous decrease in chromosome X representation. For samples in which the sex of the fetus is known or a Y chromosome elevation is observed samples from pregnant women carrying a female or a male fetus can be separated based on the chromosome Y and chromosome X representation (FIG. 72) and the fetal fraction calculated for the maternal plasma samples with a male fetus. The median fetal fraction in the capillary based maternal plasma samples with a male fetus in this study was similar, if not higher, to those observed in larger studies in venous blood, however the size of this capillary-based sample dataset is limited. Multiple maternal factors have been identified which can influence fetal fraction as it relates to NIPT testing. These include weight, body mass index (BMI) and gestational age (GA). Weight and BMI have the effect of decreasing fetal fractions levels whereas increased GA generally correlates with increased fetal fraction. FIG. 73 plots the effect of these factors on the measured fetal fraction for this capillary blood study. As seen, both weight and BMI have an inverse relationship with fetal fraction just as is the case with venous blood. Gestational age shows the expected positive correlation in capillary blood from pregnant women as has been established in venous blood. Age is not associated with a real change in fetal fraction. In this study it shows an inverse relationship, but the study size is too small to be conclusive. All trisomy samples and all euploid were correctly classified using a Z-score threshold of 3.5 in the study Z-scores for affected samples ranged from 4.6 to 18.5 and from −1.5 to 2.8 for euploid samples (FIG. 73). The resulting sensitivity in this dataset is 100% and the observed specificity is 100%. Much larger reference sample sets are normally required for accurate classification based on experience and the exceptional performance observed here indicates that this capillary blood-based methodology might outperform traditional methods. Z-scores and fetal fraction are expected to correlate, and, in this study, they did for all of the male trisomy 21 samples (n=3), see FIG. 74, lower panel.

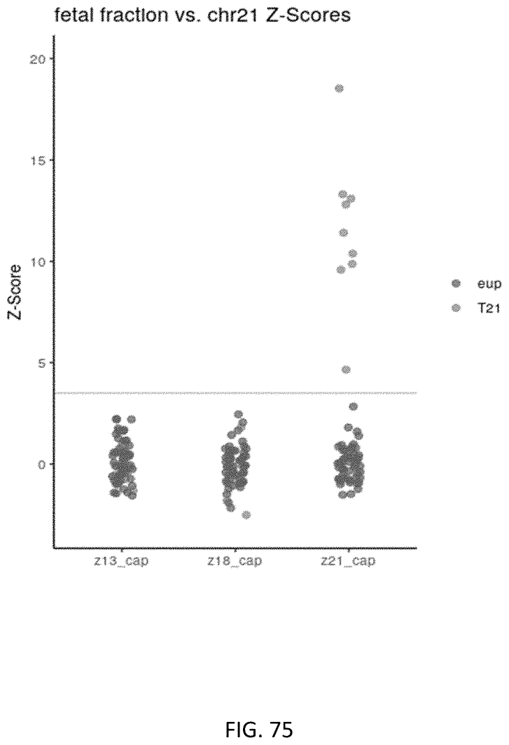

Most current NIPTs offer testing for trisomy 13 and 18 in addition to trisomy 21 and specificity should be considered as a cumulative across all tested conditions. A Z-score was also established for trisomies 13 and 18 although no samples affected by these conditions were included in the dataset. Z-scores for T13 ranged from −3.1 to 2.2 and from −3.4 to 2.5 for T18 and all were classified as negative, see FIG. 75. A specificity of 100% was achieved for testing across all three major trisomies.

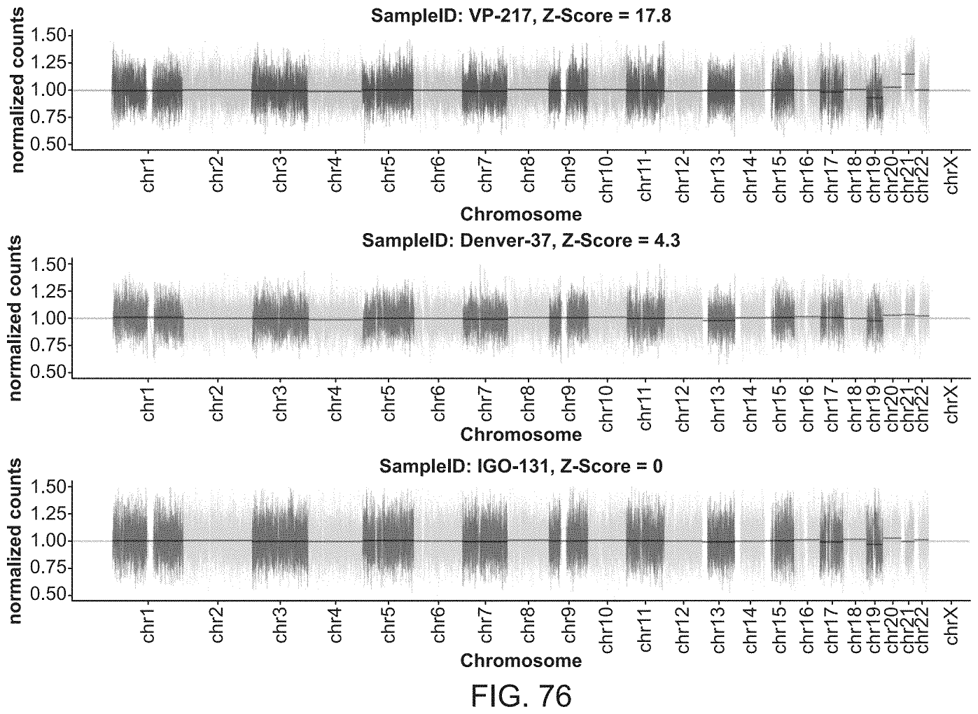

Genome-wide bin representation of capillary samples was highly similar between the three collection sites in the study. FIG. 76 shows the normalized genome-wide representation of bins per chromosome for autosomes. The plots show that most chromosomes behave as expected and have very reproducible median representations. Also, as expected, chromosome 19 is less consistent and chromosome 20 is slightly overrepresented. These factors were considered during normalization and will be further enhanced in future work.

The data from the 62 capillary blood collections in this study establish that capillary blood collected via finger prick is a viable sample type for accurate NIPT based on the ultralow input NIPT process. The cohort consisted of 53 euploid and 9 trisomy 21 samples. All aspects of the workflow including capillary sample collection and processing, library generation, normalization, pooling, sequencing and the classification pipeline were successful based upon 10 μl of maternal plasma input for ccfDNA extraction. The size distribution of capillary blood collected samples showed the expected 168 bp peak with smaller and larger ccfDNA fragments surrounding the primary mode (FIG. 71). Further investigations are required to determine the cause of the increase in these ccfDNAs in capillary blood. Fetal fraction based on Y-chromosomal representation in male samples was higher than expected which would be advantageous for NIPT testing however the size of this study is too small to be determinant and no misclassifications occurred for any samples (FIGS. 74 and 75). All 9 trisomy 21 samples were classified correctly as were all 53 euploid samples for a sensitivity and specificity of 100% in the study. Genome wide plots of normalized bin counts are as expected for samples from all three collections sites (FIG. 76). Overall, capillary blood collected via finger prick is a suitable sample type for accurate identification of trisomy 21 using the ultralow input NIPT testing process.

### Example 3: Capillary vs. Venous Blood NIPT-Equivalency

The collection of capillary blood by finger prick offers a convenient and distributable methodology for the acquisition of patient blood and blood products for biological testing at-home, at point-of-care, or at a clinical laboratory via sample shipping. As with venous collected blood, capillary blood contains circulating cell-free DNA (ccfDNA) that can be used as a biomarker for genetic testing including non-invasive prenatal testing (NIPT) as shown in examples disclosed elsewhere herein. The current standard of care sample type for NIPT is venous blood which is then processed to plasma for analysis of ccfDNA from the mother and fetus. In this study, the performance of paired capillary and venous-collected blood from pregnant females was compared in order to assess performance of the two blood collection modes as they relate to an ultralow input NIPT assay from sample collection through aneuploidy classification.

Paired capillary and venous blood samples were collected from pregnant females at three collection sites under an approved IRB protocol. A total of 62 patients participated for the study. In this sample set 9 patients carried a fetus confirmed to have trisomy 21. Amongst the non-affected samples, 3 had invasive testing results negative for Trisomy 21. The remaining 50 samples were presumed euploid samples from normal pregnancies. 20 of those samples have had cell-free DNA testing (19 low risk, 1 no results at time of blood draw), 12 patients had only ultrasound screening (US) (12 normal results) and 4 patients had both NIPT and US (all 4 were low risk and normal scan); 14 patients had no form of prenatal screening at the time of collection.

The following briefly describes the materials and methods of the study outlined above. Samples were collected under an approved IRB Protocol. Each patient had 2×10 ml EDTA blood tubes collected via venous blood draw and a minimum of 50 μl capillary blood was collected. Capillary blood was collected in an EDTA microtube.

Plasma separation was performed via centrifugation. The time from blood collection to plasma separation varied slightly between collection sites. Samples collected at Site A, were picked up and processed at the laboratory within 4 hours of blood draw. Samples collected Site B were processed on site within 4 hours of blood collection. Only samples collected at Site C were shipped overnight to the laboratory and processed within 15 hours to 24 hours.

Extraction of ccfDNA was conducted based on an input of 10 μl of maternal plasma and an elution volume of 20 μl or less.

Standard curve qPCR was conducted using a QuantStudio3 instrument and Taqman biochemistry. Assays specific for a multicopy region of the Y-chromosome and a total copy assay were used for sample quantification.

Library preparation was conducted using a library preparation kit. Library QC for size and relative concentration was conducted using a Bioanalyzer 2100 with a high-sensitivity DNA chip. Libraries were quantified for dilution and pooling using a Qubit 2.0 Fluorometer.

SBS was conducted on an Illumina NextSeq 550 with a mid-output reagent kit. Sample sheets were uploaded to a Basespace account and Fastq files were generated and stored on Basespace. Following Fastq generation the alignment and classification were uploaded to a pipeline on AWS. The pipeline was used with a z-score cutoff of 3.5 for classification of positive vs. negative Trisomy 21 and a minimum of 2 million sequenced DNA fragments which equates to 4 million reads for classification.

All samples were successfully sequenced for both capillary and venous collections based on the ultralow input workflow starting with 10 μl of maternal plasma process from blood collected in a paired fashion for capillary and venous techniques. The average number of sequenced DNA fragments was not statistically different between capillary and venous blood sources with a median of 2,812,000 for capillary and 3,748,625 for venous after alignment and QC. Both are sufficient for aneuploidy classification purposes.

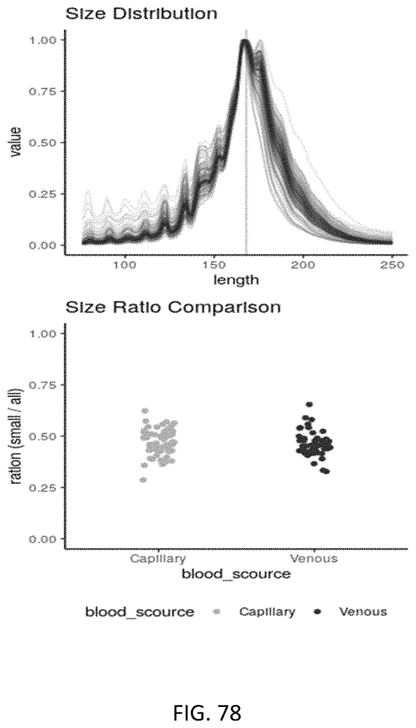

The size distributions of ccfDNA differ slightly based on the generated sequencing data with venous blood based ccfDNA centering around the mode of 168 bp, as expected for ccfDNA, compared to capillary blood based ccfDNA, which shows greater amounts of shorter and longer fragments in this data set. However, the ratio of small fragments to large fragments is not statistically different between the groups (see FIG. 78). This data confirms previous results indicating no difference between capillary and venous ccfDNA, when using specific capillary blood collection protocols and the ultralow input NIPT protocols.

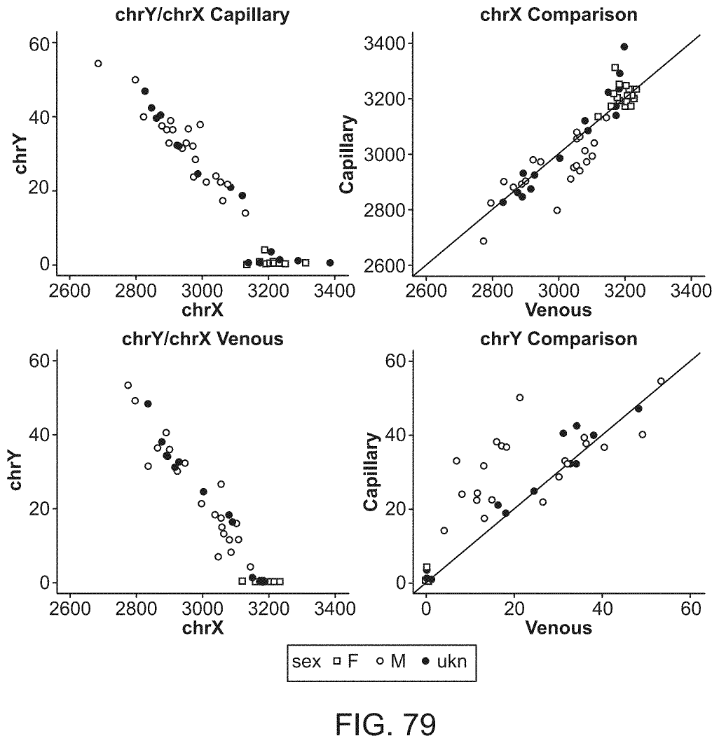

The fetal fraction as measured by the presence of Y chromosomal fragments was highly comparable between the paired sample types (see FIG. 79). Multiple capillary samples in the cohort showed a higher chromosome Y representation as seen in FIG. 79. This correlates with a higher fetal fraction, however, due the sample size it is not established that this is a real effect. Further studies with larger sample sizes will be conducted to further clarify this observation. Seven samples with no assigned fetal sex show the presence of Y chromosome in both capillary and venous samples suggesting a male fetus. There were no fundamental discrepancies between the two sample types.

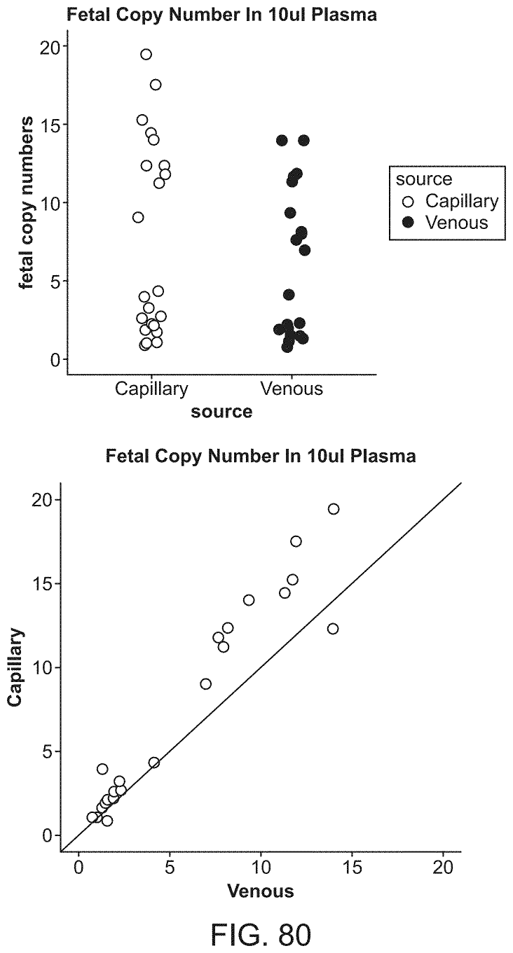

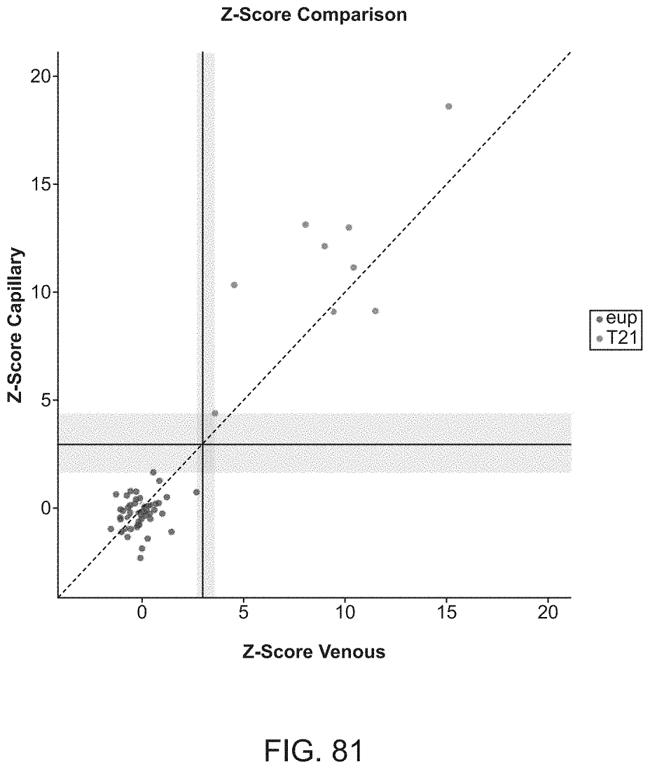

The fetal copy numbers of capillary vs. venous collected bloods based on amplification of a chromosome Y-specific multicopy region via qPCR was also measured. Developed internally, this assay uses the highly repetitive but sequence specific nature of this region to allow for detection of otherwise unquantifiable ccfDNA from ultralow volumes. As seen in FIG. 80, the fetal copies measured via qPCR correlate with the fetal fraction observations from the SBS data. Capillary collected blood shows higher amounts of fetal ccfDNA than venous blood. The two sample populations are statistically significantly different (Wilcox sign rank test: p<0.001, n=22). On average the capillary copy number per 10 μl of plasma is 35% higher than the copy number obtained from the venous sample. All values are within the expected ranges for extraction from 10 μl of plasma and ultimately provided sufficient fetal genome representation for accurate classification (see FIGS. 81-83).

FIG. 81 shows a comparison of z-scores between capillary vs. venous collected blood per subject. The comparison shows no differences in sensitivity, specificity, false positive or false negative calls between the paired collection types. The z-scores from capillary blood showed a slightly larger gap between euploid and trisomy samples (2.7 vs 0.9). This difference was largely driven by one euploid sample. A larger dataset is needed to confirm if this is a real effect overall.

In order to determine if the difference between the elevation of z-scores in capillary and venous blood was real in this study, the data was examiner further. Z-scores for euploid samples typically range from −3 to 3 and were normally distributed, however, Z-scores for trisomy samples typically range from 3 to 30 and were not normally distributed but instead depended on the fetal fraction. The two were therefore separated into distinct populations for testing. For euploids, the null hypothesis was tested that there is no difference between capillary and venous blood z-scores. The group of euploid samples was tested with a student t-test (paired and non-paired). Given the more than 50 existing data pairs the test can detect a difference of 0.5 in Z-scores with a power of 95% at a significance level of 0.05. In this data set the observed difference was −0.3 with a 95% confidence interval of −0.7 to 0, which was not statistically significant at a level of 0.05 (paired t-test) and the null hypothesis is accepted, there is no difference between capillary and venous collected samples. The group or trisomy samples can be evaluated with a Man Whitney test or a paired t-test (if the differences between capillary and venous are normally distributed). The differences between capillary and venous are normally distributed (Shapiro-Wilk normality test, p>0.05). In the 9 data pairs the one-sided paired t-test can detect a difference of −1.2 in Z-scores with a power of 95% at a significance level of 0.05. The observed mean difference between the groups was 1.7 with a 95% confidence interval from −Inf to 3.7), which was not statistically significant at the level of 0.05. (one sided, paired t-test), no difference between capillary and venous blood.

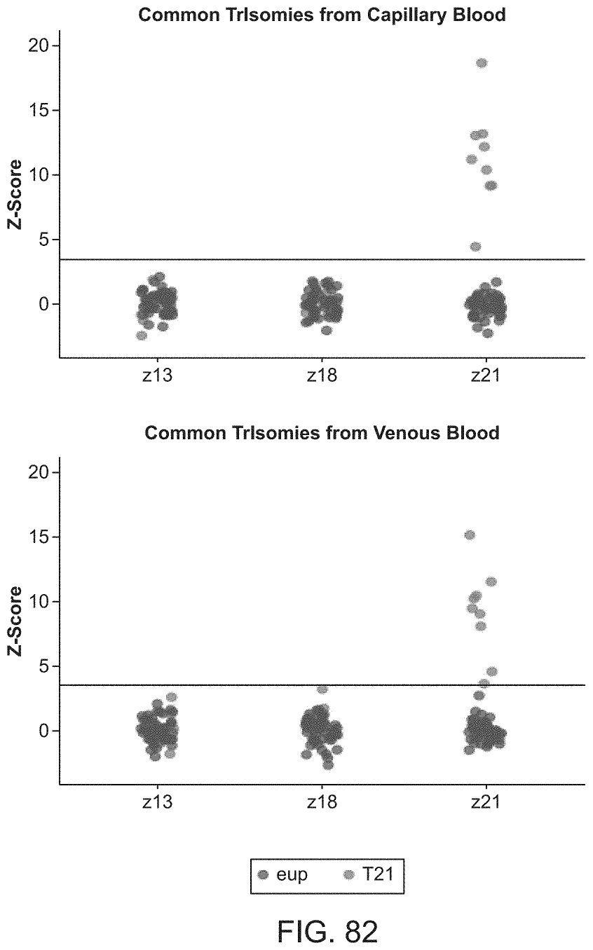

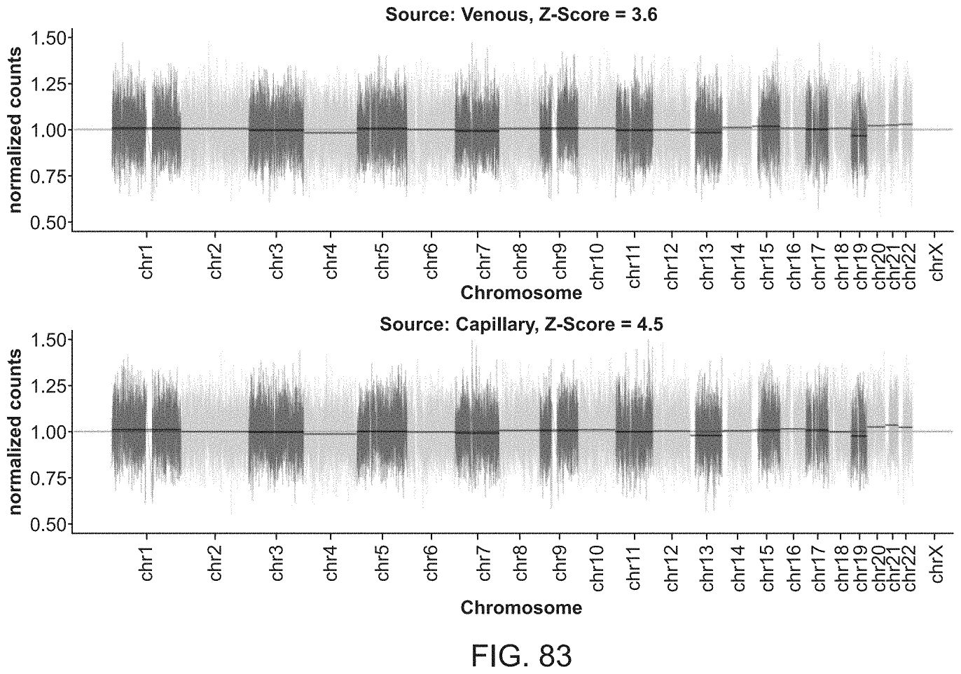

The Z-Scores for the common trisomies 13, 18 and 21 showed the expected behavior between both collection types (FIG. 82). No sample had a Z-score above the cutoff for chromosomes 13 and 18. Only samples from women carrying a fetus with trisomy 21 showed z-scores above the cutoff of 3.5 for chromosome 21, no false positives were detected. No meaningful differences between venous and capillary blood were observed. Genome-wide bin representation was highly similar between the paired collection types in the study. FIG. 83 shows the normalized genome-wide representation of bins per chromosome for the same sample within a venous and capillary collection respectively. It also shows an example of the higher elevation of counts on Chromosome 21 and higher z-score in the capillary sample.

The data from the 62 paired capillaries vs. venous blood collections in this study showed no significant differences between the two sample types based on the ultralow NIPT workflow from sample collection to classification. The cohort consisted of 53 euploid and 9 trisomy 21 samples. The size distribution of capillary blood collected samples showed slightly smaller and larger ccfDNA fragments compared to venous blood (FIG. 78) but the ratio of small-to-large fragments between the two was not different nor were the library size electropherograms or concentrations (data not shown). Fetal fraction based on Y-chromosomal representation in male samples was suggestive of a moderate increase in capillary blood-based samples which would be advantageous for NIPT testing (FIG. 79) and measured fetal copies based on Y chromosomal amplification from male fetuses correlated with this observation (FIG. 80), however the size of this study is too small to be determinant and no misclassifications occurred for either sample type (FIGS. 81 and 82). All 9 trisomy 21 samples were classified correctly as were all 53 euploid samples for both capillary and venous collections. The z-score gap was higher in the capillary blood collected samples (FIG. 81) but this difference was not found to be statistically significant. Genome wide plots of normalized bin counts appear very similar for capillary vs. venous blood collections (see FIG. 83 as an example). Overall, it was concluded that the two sample types are equivalent for the purposes of ultralow NIPT testing.

### Example 4: Capillary Blood-Collection and Characterization

Non-invasive prenatal testing (NIPT) using cell free DNA (cfDNA) and next generation sequencing became first commercially available in 2011. Since then, many clinical laboratories and commercial providers have developed and offered a variety of different technical solutions for cfDNA-based NIPT. A significant percentage of approaches use genome wide sequencing followed by alignment of the sequence reads to the human reference genome. The aligned sequence reads are then counted based on predetermined sections of the genome (typically chromosomes or sub chromosomal units called bins). Based on these bin counts a fractional representation of a predetermined target region/chromosome (for example chromosome 21) is calculated. Finally, this representation is compared to a set of normal samples. A sample is considered to be trisomic for a specified region/chromosome, if the fractional representation exceeds a predefined threshold. This calculation is often achieved by using a Z-Score calculation with cutoffs between a Z score of 3 and 4 (resulting in a specificity around 99.85%). These models work great in clinical practice and have proven practical for the last 9 years. However, there are two limitations in this approach. First, every region/chromosome requires its own Z-Score calculation. Therefore, false positive rates are cumulative over the number of regions to be tested. This is likely not a meaningful limitation when testing is limited to only Trisomies 21, 13 and 18. But it becomes increasingly relevant when testing expands beyond the common Trisomies and towards a genome wide content (for example to enable a non-invasive karyotype equivalent).

Recent advances in machine learning have not only produced previously unachieved performance in many classification tasks, but also made machine learning available to the interested public through frameworks such as tensorflow or PyTorch. An attractive feature of deep learning applications is their ability to identify feature patterns in the underlying data, and therefore enabling the removal of the constraints pertaining to highly standardized inputs. In NIPT applications it is conceivable that a neural network learns the pattern of a trisomic elevation in chromosomal sequence reads without having to predetermine which target region is to be tested. This would enable true genome-wide copy number variation (CNV) detection. Despite the promise neural network architectures hold for future classification models in NIPT, their implementation has so far not been successful due to a lack of training data. Deep learning models typically require a very large set of samples that represent each class. In the most straightforward case this means that thousands of sequencing traces are required for each of the major trisomies to be detected. This is of course unattainable since the incidence of these chromosomal abnormalities is very low (Trisomy 13 for example has an estimated incidence of 1 in 5000. It would require the results of 5M screened pregnancies to accumulate 1000 T13 positive cases). In this study, the foundational framework to build a neural network architecture was established that allows universal classification of fetal copy number variations in cfDNA. It was demonstrated that simulated data can be used for neural network training, thereby eliminating the biggest constraint for deep learning applications. It is also shown that networks can successfully predict the estimated magnitude of the copy number variation and that using chromosome specific quantitative information combined with sample specific metainformation can be used to successfully classify NIPT samples.

A set of 13×96-well plates comprising a total of over 1200 clinical samples were sequenced. From this sample set, three datasets were constructed: a training set, a test set and a validation set. 96-well plates 1 to 7 were used to build a training and test set, while 96-well plates 8 to 13 were set aside as a completely independent validation data set. The construction of the training and test set is fundamental for the study and is therefore described in more detail in the following paragraphs.

Probability tensors were constructed based on the unaligned sequence reads for all samples from plates 1-7. Loss correction on each tensor to account for GC sequencing bias was also performed. For each bin in the normalized probability tensors a mean and a standard deviation was calculated across a set of euploid samples. A set of bins (here n=10,000) with a low standard deviation and a low difference from the overall median of 1 were select for chromosomes 21, 13, 18 and from chromosomes not affected by the three main trisomies (1, 2, 3, 4, 5, 6, 7, 8, 9, 10, 11, 12, 14, 15, 16, 17, 20, 22). To build the training set of simulated samples, first the number of requested training samples was determined (here n=10,000). Next for each sample in the training set a virtual fetal fraction was assigned. The assignment of fetal fraction was based on a random sampling from a beta distribution (which is known to model the fetal fraction distribution in a clinically representative cohort). Samples with fetal fraction assignments lower than 5% were elevated to 5% to increase the model's chances of successful training. Each sample was then assigned a class from the set of euploid (50% of the samples), trisomy 21, trisomy 13 or trisomy 18 (⅙th of the simulated samples each). For each training sample a simulated probability tensor was generated by sampling the above mentioned 10,000 bins based on the previously determined mean and standard deviation for each bin. Finally, for each sample that was assigned a trisomy class, the bins in the simulated probability tensor representing the respective trisomy were elevated proportional to the assigned fetal fraction. For example, a sample with the assigned class trisomy 21 and a fetal fraction of 9.8%, will have the bin values originating from chromosome 21 elevated by an average of 4.9%. Because the measurement variance is already captured in the original simulated probability tensor, it is sufficient to elevate the bin values by a constant proportion. This method ultimately provides a set of simulated training samples for which each class assignment corresponds with a proportional increase in associated bins. To prepare the input data for neural network training, the data for each sample was transformed into a 2 dimensional 100 by 100 input tensor. The neural networks were then trained based solely on this simulated data.

To create the test set, the same normalized probability tensors as described previously were used. However, unlike the simulated dataset this data is based on actual sequencing data. The existing data was simply transformed into a 2D tensor according to the same procedure used for the training set. This difference is very important. While the training set consisted exclusively of artificial data that had never existed in the real world, the test set data is real sequencing data from plates 1-7, but it is slightly rearranged to fit the classification input tensors. However, because the training data is derived from the means and standard deviations obtained from plates 1-7, it can be argued that test and training data are not completely independent data sets. To overcome this limitation, a fully independent validation set was also sequestered.

The generation of the validation set followed the same procedure as the test set, but instead of using samples from plates 1-7, the only samples included in the validation set were samples from plates 8-13. This made the validation set completely independent from the training data set.

For each of the trisomy classes a network was trained to provide a numerical value that is intended to be above a threshold in case the sample is of the affected class and is proportional to the magnitude of the injected bin elevation. These properties are represented by a Z-score in the traditional methodology and hence a Z-score can be used as a training metric (a similarly good metric would have been the fetal fraction or chromosomal representation). However, because the data tensor is reduced to include 10,000 bins the Z-score calculation was modified to work with the reduced data tensor. It is important to note that these “training Z-scores” are not equal to the traditional Z-scores. Those traditional Z-scores were calculated based on the majority of all bins representing autosomes. In this report the Z-scores based on the selected 10 k bins are referred to as “TMZ-scores” (Training Material Z-scores) and traditional Z-scores are referred to as “Z-scores”. Scores obtained by the neural network will be referenced as P-scores (Predicted Scores).

For each of the affected classes (trisomy 21, trisomy 13 and trisomy 18) a convolutional neural network was trained using the TMZ-scores as the optimization goal for regression. The convolutional neural network (CNN) architecture was identical for all three classes, but after training of course the weights and biases changed to best predict the TMZ score for each class. After training, P-scores were then calculated for the entire training set. To train a classification network the P-scores were combined with the assigned fetal fraction into one 1D input tensor. The entire process was compiled into one larger network by the use of custom layers. This study is a proof-of-concept study and therefore it is prudent to break out each step of the process and investigate the individual contributions of each step.

The following paragraph(s) describe the materials and methods utilized for the aforementioned study.

NIPT samples: Over 1200 de-identified and research authorized pregnant female plasma samples that have previously undergone NIPT were analyzed. The samples were collected via standard NIPT venous blood collection between the gestational ages of 9-30+ weeks with a median of 12 weeks. Plasma separation was performed at a clinical lab.

Extraction of Cell-Free DNA (cfDNA): Extraction of cfDNA was conducted based on an input of 16 μl of maternal plasma and an elution volume of 20 μl.

Library preparation was conducted using a library preparation kit. Library QC for size and relative concentration was conducted using a Bioanalyzer 2100 with a high-sensitivity DNA chip. Libraries were quantified for dilution and pooling using a Qubit 2.0 Fluorometer.

Sequencing-by-Synthesis (SBS) and Analysis: SBS was conducted on an Illumina NextSeq 550 with high-output reagent kits. Sample sheets were uploaded to a Basespace account and Fastq files were generated and stored on Basespace.

Probability vector generation: The fastq files were transferred from ILMN Basespace to an AWS-S3 instance and processed using a EC2 instance running a proprietary pipeline for non-alignment. (c_pipe). The pipeline transforms each fastq file into a probability vector across 64455 preassigned chromosome annotations.

Neural network Training: The linear probability tensor was transformed into a 2D input tensor of shape 100*100, containing 500 chr21 bins, 1200 chr 18 bins and 1500 chr 13 bins, as well as 6800 bins from other autosomes (excluding chr 19). The previously calculated TMZ scores were used as an optimization target for regression.

P-score regression: A convolutional neural network with a convolution layer that accepts the 100*100 2D input tensor was used (FIGS. 37 and 38). The first layer contained 100 layers at a kernel size of 5*5. The next convolutional layer contained 200 filters at a kernel size of 3*3. These convolution layers were followed by a max pooling layer (pool size=3*3, a dropout layer (rate=0.25) and a flattening layer. Next were two fully connected layers (first 256 units, second 128 units) each were followed by a dropout layer (rate=0.5). Finally, a single unit was used for outputting the predicted value. Activities were relu for the convolutional layers and leakyrelu for the fully connected layers. The final unit had linear activation.

Training was performed for at least 50 epochs with a batch size of 500 samples. The data was split 80/20 for training/validation (here “validation” is a tensorflow term used for the validation split of the training data).

Classification: All three models were run on the training set and the P-scores were combined with the assigned “fetal-fraction”. This network was a fully connected network as a classification network. This led to a 1D input tensor of length 4. The first layer contained 8 units, the second layer contained 4 units and the final layer also contained 4 units. Activation was relu for the first two layers and softmax for the final layer. The optimization target was the class value that was assigned during the generation of the training set. Classification was performed by assigning a class value to any sample that had a softmax score above 0.9 for any class, while for a sample that did not achieve that level of certainty a “no-call” was assigned.

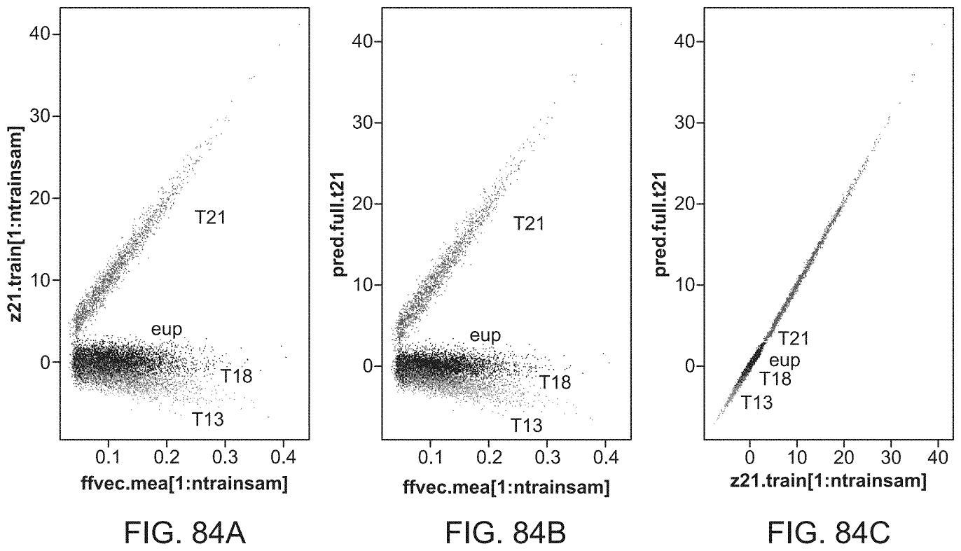

The models for all three classes trained successfully over 50 epochs. The final mean absolute errors for the predicted Z score of a sample were below 0.7 for the 80% training portion of the training set and surprisingly below 0.3 for the validation portion of the training set (Table 2). As indicated by the low mean absolute errors, the difference between TMZ-scores and P-scores is small. In addition, TMZ-scores as well as P-scores show the expected relationship with assigned “fetal fraction” (FIGS. 84A-84C, 85A-85C, and 86A-86C).

FIGS. 84A-84C show the relationship between the assigned fetal fraction and the TMZ score (FIG. 84A), the P-score (FIG. 84B), and the relationship between the TMZ score and the P-score (FIG. 84C). Samples were assigned a T21 class, a T13 class, a T18 class, and no elevation assignment (euploid), and are depicted in FIGS. 84A-84C.

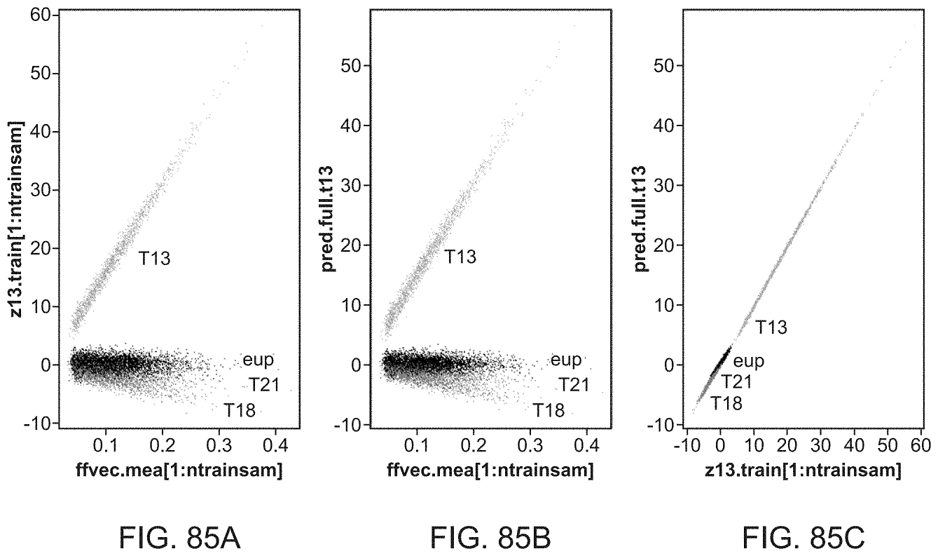

FIGS. 85A-85C show training set results for ConVNet13. Relationships between the assigned fetal fraction and the TMZ score (FIG. 85A), the P-score (FIG. 85B), and the relationship between the TMZ score and P-score (FIG. 85C). Samples were assigned a T21 class, a T13 class, a T18 class, and no elevation assignment (euploid), and are depicted in FIGS. 85A-85C.

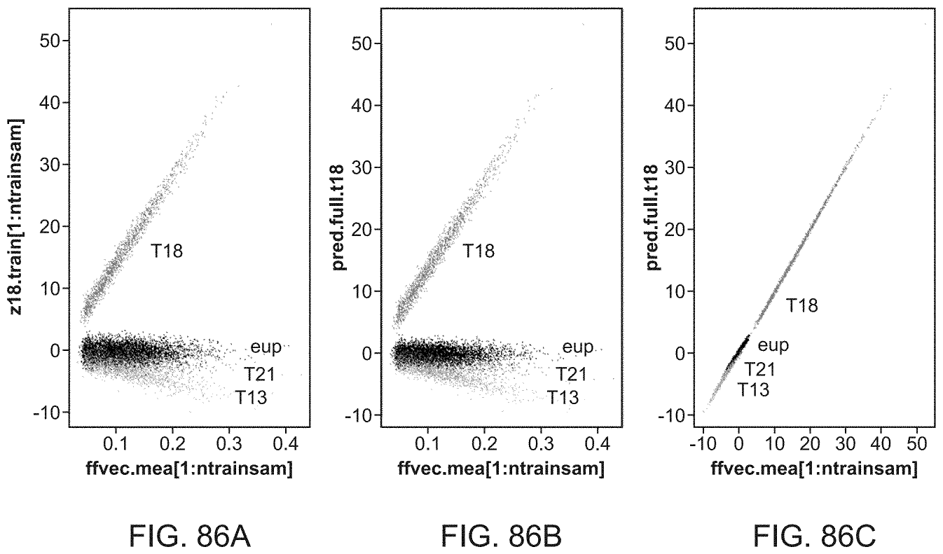

FIGS. 86A-86C show training set results for ConVNet18. Relationships between the assigned fetal fraction and the TMZ score (FIG. 86A), the P-score (FIG. 86B), and the relationship between the TMZ score and P-score (FIG. 86C). Samples were assigned a T21 class, a T13 class, a T18 class, and no elevation assignment (euploid), and are depicted in FIGS. 86A-86C.

TABLE 2
Training and validation error after successful training on
10000 samples for 50 epochs

| | Mean absolute error- | Mean absolute error- |
|---|---|---|
| | training portion | validation portion |
| ConVNet21 | 0.49 | 0.21 |
| ConVNet13 | 0.63 | 0.28 |
| ConVNet18 | 0.65 | 0.23 |

The classification model was trained over 2000 epochs, using the same training validation split of 80 to 20. The final accuracy was 99.84% for the training portion of the data and 99.9% for the validation portion of the data for Trisomies 21, 18 and 13. This is reflected in the softmax scores, which show that trisomies 13 and 18 are slightly more distinct from all other groups than trisomy 21. To further evaluate performance, assigned class was compared to classifications resulting from the TMZ-score and the P-score (Tables 3-5).

First, it was necessary to evaluate how well the TMZ-score corresponded to the assigned class. In total the data from the 10,000 training samples showed 9 false positives (on approximately 5000 euploids) and 11 false negatives (out of approximately 5000 affected). Comparing the P-scores to the assigned class revealed a similar picture. No false positives were detected and only 10 false negatives were observed. In addition, because the classification allowed for a no call option, 23 samples were called “no-call”.

The most relevant comparison for this study was the comparison of the gold standard method (Z-score) with the novel network-based method. Z-score based classification rules from the Clinical Performance Verification Study were applied to this dataset of TMZ-scores. This comparison showed an extremely high correlation between the two classification methods. A total of 58 samples were in the no-call category for the TMZ-score based classification, while only 23 samples were in the no-call category for the P-score base classification. The relative high proportion of no-call samples in the TMZ-score classification is explained by the overrepresentation of low fetal fraction samples in this dataset.

In conclusion, this data shows that the P-score classification has the potential to reduce inconclusive samples in a meaningful way.

TABLE 3
Cross table of class assignment (in rows, [euploid, Trisomy13, Trisomy18, Trisomy 21]) and TMZ-score
based calling (in columns, [eup_z, t13_z, t18_z, t21_z])

| class | eup_z | t13_z | t18_z | t21_z |
|---|---|---|---|---|
| euploid | 4993 | 0 | 0 | 9 |
| Trisomy 13 | 1 | 1696 | 0 | 0 |
| Trisomy 18 | 1 | 0 | 1619 | 0 |
| Trisomy 21 | 9 | 0 | 0 | 1672 |

TABLE 4
Cross table of class assignment (in rows, [euploid, Trisomy13, Trisomy18, Trisomy 21]) and P-score
based calling (in columns, [eup_p, no_call, t13_p, t18_p, t21_p])

| class | eup_p | no_call | t13 _p | t18_p | t21_p |
|---|---|---|---|---|---|
| euploid | 4997 | 5 | 0 | 0 | 0 |
| Trisomy 13 | 1 | 1 | 1695 | | 0 |
| Trisomy 18 | 0 | 4 | 0 | 1616 | 0 |
| Trisomy 21 | 9 | 13 | 0 | 0 | 1659 |

TABLE 5
Cross table of TMZ-score based calling including an indeterminant
zone (in columns, [eup_z, noc_call, t13_z, t18_z, t21_z]) and P-score
based calling (in columns, [eup_p, n_call, t13_p, t18_p, t21_p])

| class | eup_p | no_call | t13 _p | t18_p | t21_p |
|---|---|---|---|---|---|
| eup_z | 4969 | 0 | 0 | 0 | 0 |
| no_call | 38 | 15 | 2 | 0 | 3 |
| t13_z | 0 | 1 | 1693 | 0 | 0 |
| t18_z | 0 | 3 | 0 | 1616 | 0 |
| t21_z | 0 | 4 | 0 | 0 | 1656 |

For the evaluation of the P-scores and the associated classification results in the test and validation set, TMZ-scores are not the most relevant. Instead, the actual Z-scores obtained during the Clinical Performance Verification Study will be used. These are more appropriate when moving from simulated data to real world data and should correspondingly evaluate real world results as well.

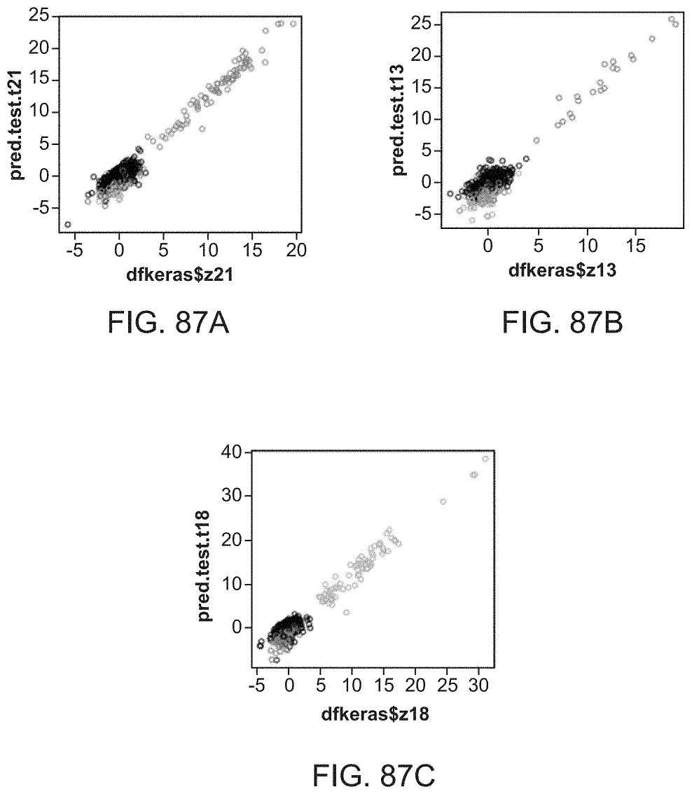

Even though Z-scores were calculated based on the entire dataset and P-scores were based on only a subset of 10,000 bins, the correlations between Z-score and P-score were very high for the test set data (FIGS. 87A-87C). FIGS. 87A-87C show the relationship between Z-score obtained in the Clinical Performance Verification study and P-scores derived from the ConVNet prediction for the test samples. Trisomy 21 (FIG. 87A); Trisomy 13 (FIG. 87B); Trisomy 18 (FIG. 87C). Trisomy 21 samples are colored blue, Trisomy 13 samples are colored red, Trisomy 18 samples are colored green and euploid samples are colored black.

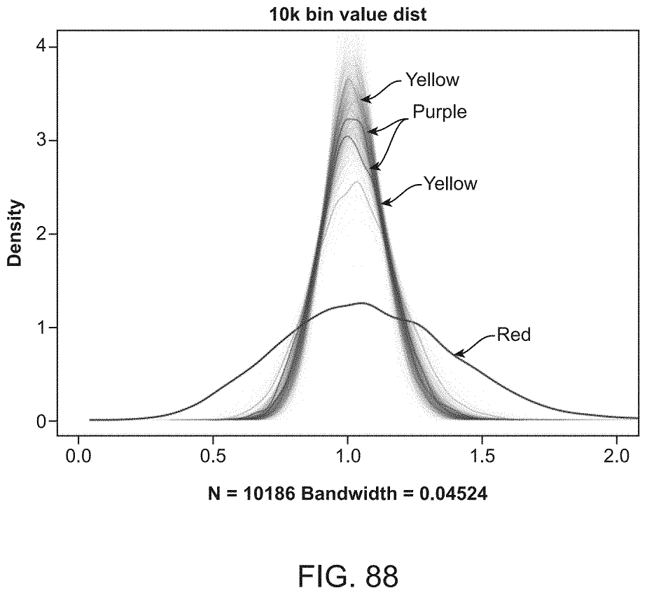

As a basis for comparison the Z-score based classification from the Clinical Performance Verification Study was used to evaluate the P-score based classification (Table 6). Interestingly two samples that were classified as euploid based on the Z-score classification were classified as T21 based on the P-score classification. In addition, 1 sample was classified as T18 based on Z-score but identified as euploid based on the P-score. Upon closer inspection the two “false positive” samples have low p-scores and would likely be identified during a lab director review and flagged. The “false negative” T18 sample is even more interesting. When inspecting the underlying data of this sample it is immediately evident that the distribution of bin values in the probability tensor is much wider compared to the rest of the sample set (FIG. 88). FIG. 88 shows a density plot of bin value distributions for the 10000 bins in the probability tensor. Each line represents one sample in the test. Orange are samples that were called euploid in the Clinical Performance Verification Study and called no call using the P-score. Purple are samples that were called euploid in the Clinical Performance Verification Study and called T21 using the P-score. The sample in red was called T18 in the Clinical Performance Verification Study and called euploid using the P-score. Because this set was trained based on simulated data, the training data did not include sufficient observations to train the model on this kind of outlier behavior. However, an additional quality control parameter is easily established to prevent these kinds of errors going forward. Again, it was observed that the number of inconclusive samples is less in the P-score based classification model

TABLE 6
Cross table for the test set samples. Aneuploidy calls from the Clinical
Performance Verification Study (in columns, [EUP, Inconclusive, T13, T18, T21]) and P-score based calling
(in columns, [eup_p, no_call, t13_p, t18_p, t21_p])

| | eup_p | nocall | t13 _p | t18_p | t21_p |
|---|---|---|---|---|---|
| EUP | 480 | 2 | 0 | 0 | 2 |
| Inconclusive | 2 | 0 | 1 | 0 | 1 |
| T13 | 0 | 0 | 20 | 0 | 0 |
| T18 | 1 | 0 | 0 | 73 | 0 |
| T21 | 0 | 0 | 0 | 0 | 83 |

Overall, the results from the completely independent validation set confirm the results from the test set data.

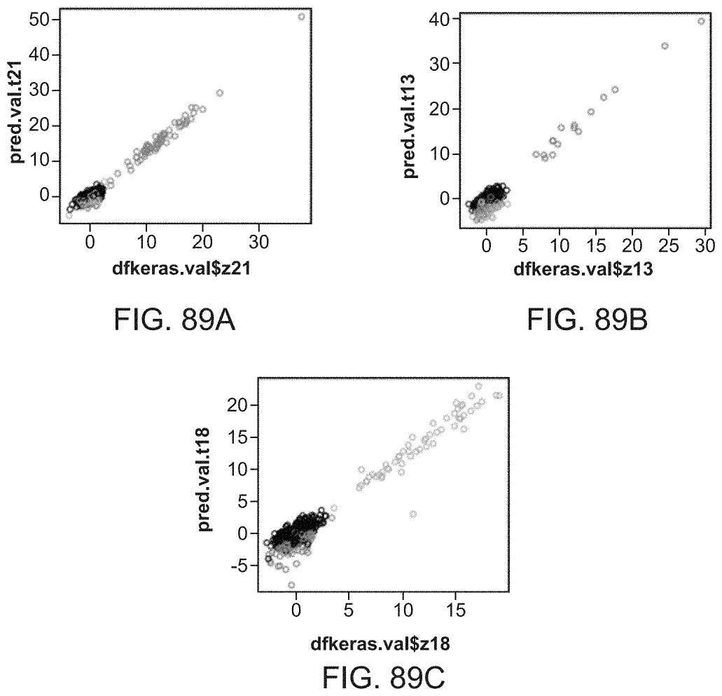

There is a high correlation between Z-score from the original Clinical Performance Verification Study and the P-scores (FIGS. 89A-89C). This confirms that trained regression models are universally applicable and are valid on independent data sets, a highly important result. FIGS. 89A-89C show the relationship between Z-score obtained in the Clinical Performance Verification Study and P-scores derived from the ConVNet prediction for the validation set samples. Trisomy 21 (FIG. 89A), Trisomy 13 (FIG. 89B), and Trisomy 18 (FIG. 89C). Trisomy 21 samples are colored blue, Trisomy 13 samples are colored red, Trisomy 18 samples are colored green and euploid samples are colored black.

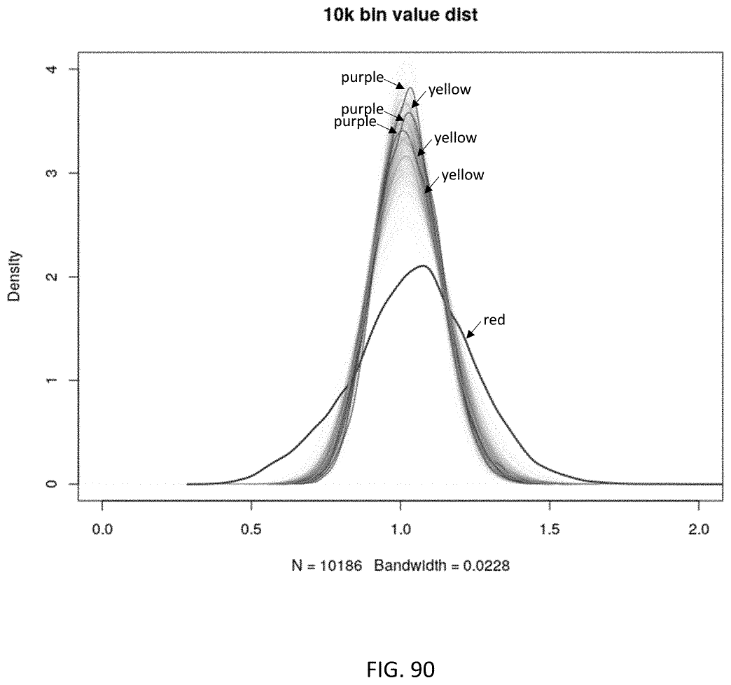

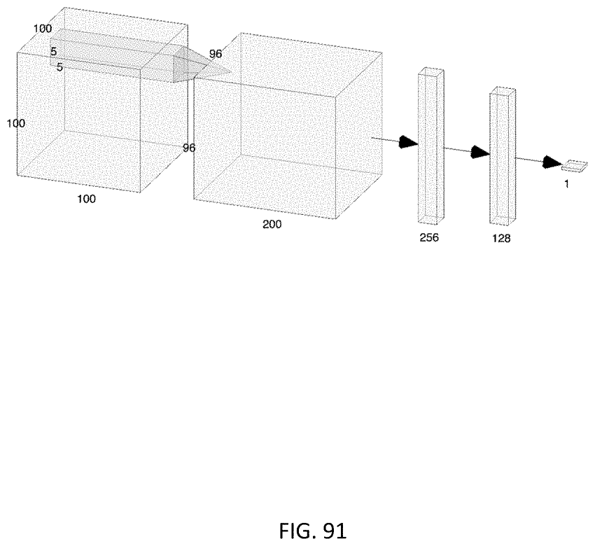

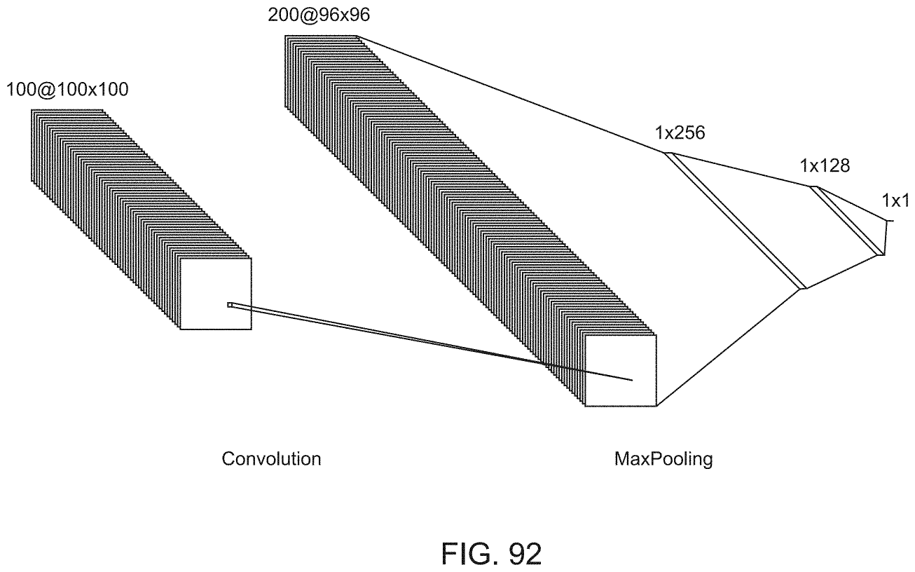

The classification of the completely independent validation set confirmed the high performance of the classification results in the test set (Table 7). Interestingly, one sample in the validation set showed the same behavior (aberrant bin distribution) as the misclassified T18 sample from the test set (FIG. 90). FIG. 90 shows a density plot of bin value distributions for the 10000 bins in the probability tensor. Each line represents one sample in the validation set. Orange are samples that were called euploid in the Clinical Performance Verification Study and called no_call using the P-score. Purple are samples that were called euploid in the Clinical Performance Verification Study and called T21 using the P-score. The sample in red was called T18 in the Clinical Performance Verification study and called euploid using the P-score. Therefore, this sample would be retested in a real-life scenario before resulting the sample out. Again, one sample that has been classified as euploid based on the Z-score is identified as T21 based on the P-score. But also, just as in the test set, this sample has a low P-score (3.4) and would be flagged for follow-up in clinical practice. Both sets include 3 no-call samples.

TABLE 7
Cross table for the validation set samples. Aneuploidy calls from the
Clinical Performance Verification Study (in columns, [EUP, Inconclusive, T13, T18, T21]) and P-score
based calling (in columns, [eup_p, no_call, t13_p, t18_p, t21_p])

| | eup_p | no_call | t13_p | t18_p | t21_p |
|---|---|---|---|---|---|
| EUP | 387 | 2 | 0 | 0 | 1 |
| Inconclusive | 1 | 1 | 0 | 0 | 1 |
| T13 | 0 | 0 | 16 | 0 | 0 |
| T18 | 1 | 0 | 0 | 54 | 0 |
| T21 | 0 | 0 | 0 | 0 | 69 |

Adoption of neural network models in biological sciences has been slow compared to more engineering-based disciplines. One reason for this delay is the relative scarcity of well annotated biological data. While there exist many very large datasets for training neural networks on tasks like image detection, the availability of biological datasets is scarce. Furthermore, for many biological tasks defining ground truth is much more complex. In medical science deep learning methods are often deployed for specific diagnostic tasks, such as mammography interpretation. Their performance is typically evaluated against a panel of experts, because it has been shown that a single physician classifying images is an insufficient source. Digitalization has been adopted for a while in medical imaging, but molecular diagnostics as a field is further behind. While some repositories exist that hold sufficiently large sequencing datasets, their annotation is in its infancy. Therefore, it has been long believed that using deep learning methods in molecular diagnostics will be impaired by the unavailability of sufficiently annotated datasets. To make inroads into this field, we selected a specific application with well-defined class labels and a well-established relationship between data and its interpretation. In cfDNA-based NIPT the overrepresentation of a set of genomic regions indicates a fetal copy number variation at said region, assuming an euploid status in the mother. Detection of this overrepresentation is a task that can be captured numerically and hence is accessible for deep learning models. To overcome the reference material limitations, a novel method for designing training data sets was developed. When analyzing the data of existing data sets, they are found to be reasonably stable and hence rules can be established to build large in-silico datasets for network training. This strategy proved very successful. Not only was very high performance achieved in the simulated training data, that performance in the test data set and the completely independent validation data set was confirmed. The segregation of the data into three datasets is another strength of this study. Separation of training and test set data is best practice in machine learning applications. Typically, these are derived from the same pool of data and then often split prior to training. In this study the training data was generated entirely in-silico with no real-life correlate. The test and validation data however were derived from actual physical experiments. The fact that the model can be trained on simulated data and then applied to real-life data with high performance underlines the strength of the approach.

It is important to note that this study is a first implementation of the deep learning model to an NIPT classification problem. It is self-evident that this approach will become better over time. This is especially relevant for two cases. Two samples were observed (one in the test set and one in the validation set) where the distribution of bin values did not match the expected distribution of values. Therefore, the model's training was unprepared to classify these samples. For now, this behavior can be corrected by introducing a simple quality control check, similar to a fetal fraction or sequence read cutoff, that would flag these samples for retesting. In the future these samples will add to the pool of available training data and the model will learn to automatically identify them for retesting. The ability to continuously add new information to the model and improve its performance over time is one of the most compelling reasons to shift away from a deterministic approach like Z-score's towards a dynamic approach like convolutional neural networks.

When these models are first implemented to clinical practice it might be prudent to implement a heuristic that favors stronger lab director involvement. In clinical laboratories it is common practice for the lab director to batch sign out all negative results and to review all positive results. Typically, the lab director will order samples that are close to a Z-score cutoff to be retested. Would such a method be implemented on the results obtained in this study, it would safely identify all samples that had been labeled trisomic by the ConVNet and inconclusive or euploid by the Z-score method, thereby minimizing the risk of false positive results.

This is an exciting step and encourages more work on NIPT classification models. Future work will include generalized CNV detection models, which enables karyotype like resolution and adding additional clinical parameters to the classification model. There are two types of additional information that might benefit the classification model. First, technical information about the sequencing run, such as total sequenced DNA fragments, bin variance, GC bias etc. With enough information the model will likely be able to determine an equivalent of a QC parameter. Second, clinical information. Currently the model only uses fetal fraction, but it is well known that parameters such as Age, BMI or gestational age can influence the prior in traditional statistical models. With a large enough real dataset these effects should benefit the model.

In summary it is concluded that convolutional neural networks can reliably identify fetal trisomies 21, 13 and 18 from standard whole genome sequencing NIPT data. The performance is on par with traditional approaches and provides the benefit of being able to evolve as more data becomes available.

### Example 5: Ultra-Low Input NIPT-Clinical Performance Verification

Genetic testing workflows amenable to ultra-low input amounts of nucleic acids to enable self and point-of-care sample collections such as capillary blood by finger prick are being developed. Such collections offer a convenient and distributable methodology for the acquisition of patient blood and blood products for biological testing. In order to make this possible, testing workflows have been optimized to perform robustly with very low input amounts of template for enzymatic conversion and amplification of target molecules. Circulating cell-free DNA (ccfDNA) is found in blood and it is used for multiple diagnostic applications such as non-invasive prenatal testing (NIPT). The current standard-of-care sample type for NIPT involves large volumes of venous blood collected by a phlebotomist which is then shipped to a clinical lab for processing and analysis. The general workflow involves processing the blood to plasma, extraction and purification of ccfDNA, generating a sequencing library of unique target nucleic acids, sequencing of the targets and a bioinformatics pipeline for analysis and classification of each sample. The methods and systems described herein have optimized all steps of the NIPT workflow to accommodate ultra-low inputs well below what is standardly used for NIPT. Previous studies have established the analytical and clinical feasibility of the ultra-low input NIPT process in both capillary and venous blood along with their equivalency in data produced by the two collection types. This study looks to verify the clinical performance of the ultra-low input NIPT process in a large clinical sample cohort of over 1200 plasma aliquots from pregnant females and contains a high proportion of positives for the most common fetal trisomies, sex-chromosome aneuploidies and micro-deletions.

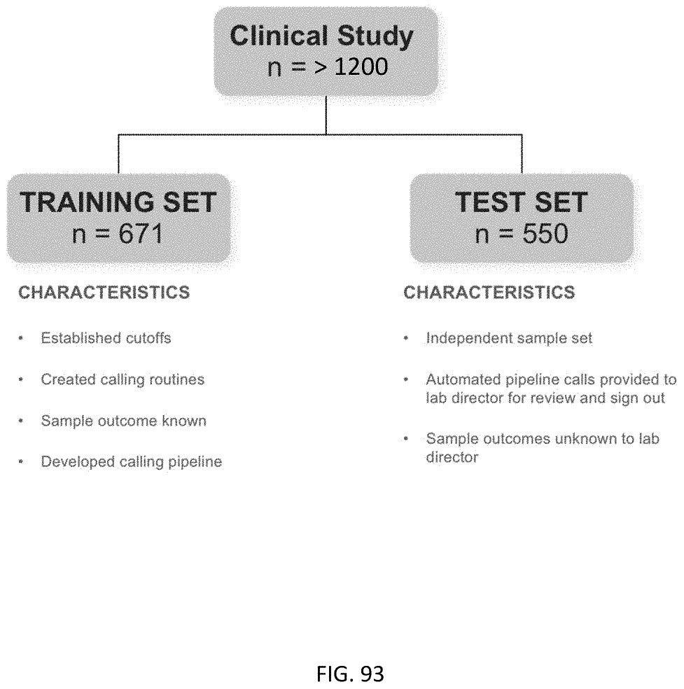

Clinical performance verification of the ultra-low input NIPT process was assessed using previously tested venous-collected maternal blood samples from pregnant women provided by a commercial NIPT clinical lab under MTA. The overall sample cohort consisted of over 1200 samples with 300 common trisomies, over 190 fetal sex chromosomal aneuploidies and 20 less common events in all composed of 159 fetal trisomy 21 (n=159), fetal trisomy 18 (n=130), fetal trisomy 13 (n=37), fetal trisomy 22 (n=9), fetal trisomy 16 (n=9), sex chromosomal aneuploidies (n=197), micro-deletions (n=12) and normal euploids (n=668) with 54.6% of the samples containing a Y chromosome. The study was designed into a training set of n=671 samples to optimize the process, especially the classification pipeline and a blinded test set of n=550 to assess performance of the optimized process from extraction through classification (FIG. 93). The test set data assessment and final call was conducted by a blinded individual and the results submitted for unblinding in order to mimic the NIPT process in a licensed clinical lab.

Extraction of Circulating Cell-Free DNA (ccfDNA): Extraction of ccfDNA was conducted based on an input of 16 μl of maternal plasma and an elution volume of 20 μl.

Whole Genome Library Preparation: Library preparation was conducted using a library preparation kit. Library QC for size and relative concentration was conducted using a Bioanalyzer 2100 with a high-sensitivity DNA chip. Libraries were quantified for dilution and pooling using a Qubit 2.0 Fluorometer.

Sequencing-by-Synthesis (SBS) and Analysis: SBS was conducted on an Illumina NextSeq 550 with high-output reagent kits. Sample sheets were uploaded to a Basespace account and Fastq files were generated and stored on Basespace. Following Fastq generation the alignment and classification were uploaded to a pipeline on AWS. The pipeline was used in this study for classification of positive vs. negative trisomies, sex chromosomal aneuploidies and microdeletions. A minimum of 2 million sequenced ccfDNA fragments which equates to 4 million reads was required for classification acceptance. Cutoffs for common trisomies in the test set were as follows: trisomy 21 positive: Z-score>/=3.5, trisomy 18 positive: Z-score>/=4.5, trisomy 13 positive: Z-score>/=5. Sex chromosomal aneuploidies are calls were based on the Y-chromosome and the X-chromosome representation. Female samples are expected to not show any Y-chromosome and their X-chromosome representation is expected to follow a normal distribution comparable to percentage representations for autosomes. Female samples and female sex chromosome aneuploidies can be classified using the X-chromosome representation. Male samples do have a Y-chromosome representation and show a strong correlation between decrease in X-chromosome representation and increase in Y-chromosome representation. This relationship can be measured and outliers can be determined. Performance characteristics were evaluated based on the test-results provided by the commercial laboratory. Sensitivity and Specificity were calculated for the common trisomies individually (trisomy 21, trisomy 13, trisomy 18) and sex chromosomal aneuploidies in aggregate. In order to replicate a clinical lab NIPT process the automated pipeline calls for the test set were provided to an independent blinded clinical lab scientist (CLS) for data review and sign out of the final call. The CLS could sign out the call, request a retest (sample gets processed from plasma again, request a rerun or sign the sample out as non-reportable.

NIPT samples: Over 1200 de-identified and research authorized pregnant female plasma samples that have previously undergone NIPT were obtained. The samples were collected via standard NIPT venous blood collection between the gestational ages of 9-30+ weeks with a median of 12 weeks. Plasma separation was performed at their clinical lab.

The samples in this study were randomly assigned into two groups (FIG. 93): (1) The training set (n=671) set was used to establish a data pipeline, determine quality cutoffs, refine calling algorithms and set calling rules to be used for aneuploidy calling; (2) The test set (n=550) was processed using the data processing pipeline and calling rules established from the training set.

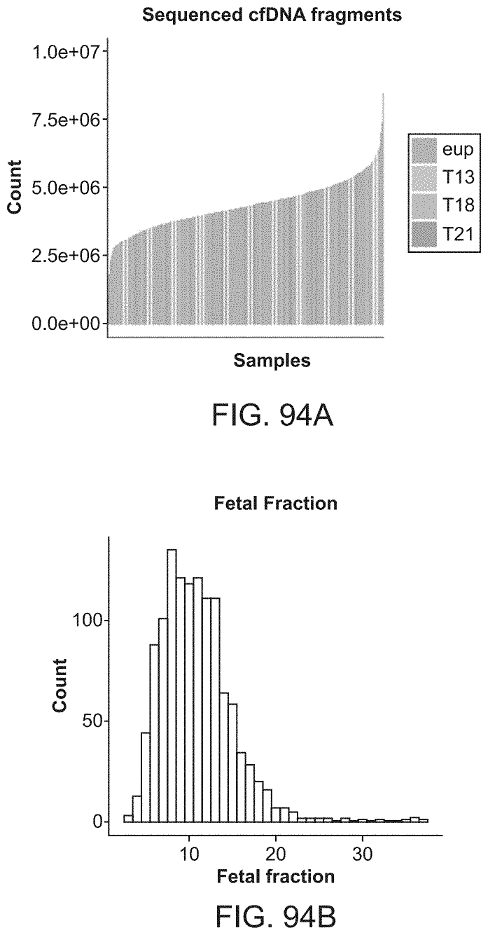

Sequencing yielded a median of 4.3 million sequenced ccfDNA fragments per sample for both the training and test sets of samples (FIGS. 94A and 94B). FIG. 94A shows sequenced ccfDNA fragments for the study with a median value of 4.3 million per sample. FIG. 94B shows Fetal fraction as measured from the training and test sets, 10.1% and 10.8%, respectively. Six samples out of 571 in the training set did not meet the minimum of 2 million sequenced ccfDNA fragments (1.0%) and were failed. These 6 samples included one trisomy 13 and one trisomy 21 sample.

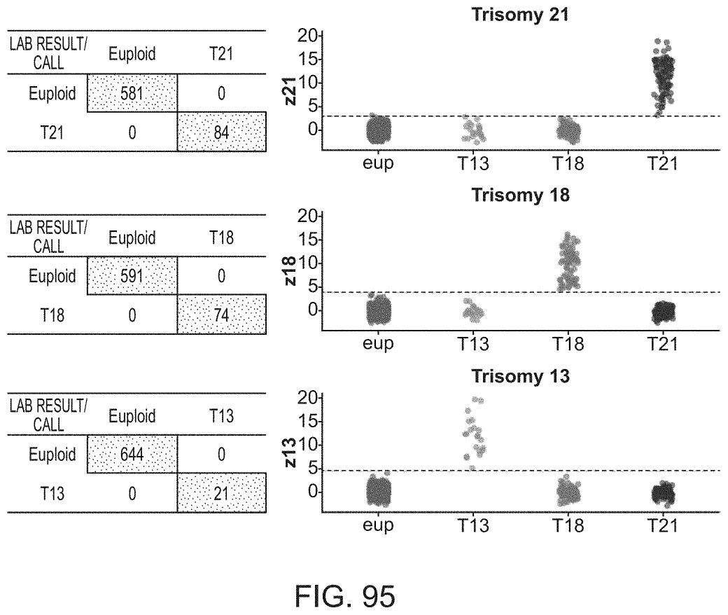

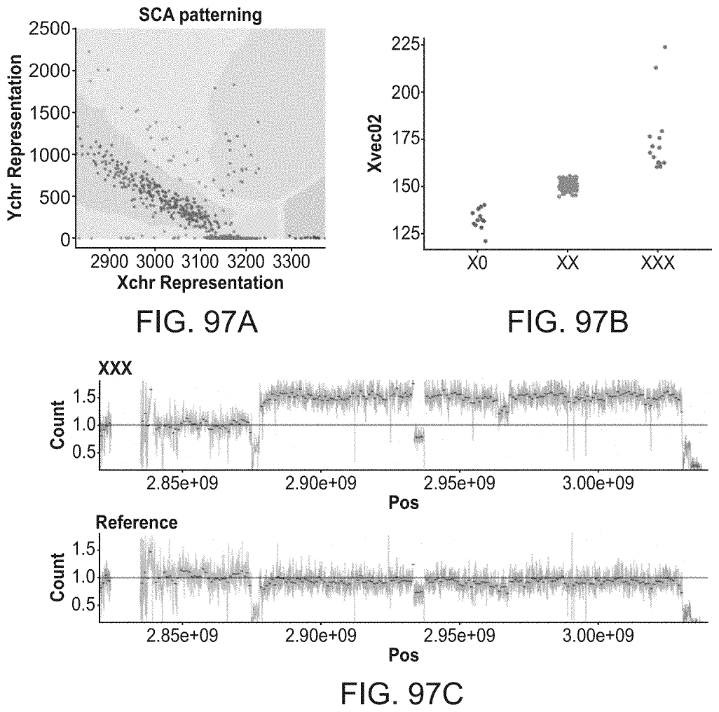

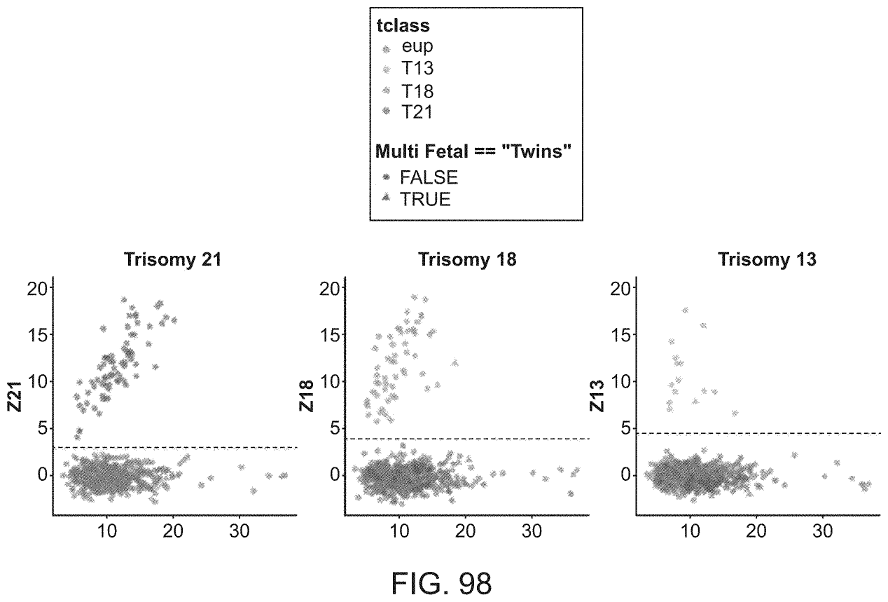

For the training set of samples, the automated pipeline calls were provided to a reviewer. The reviewer examined available clinical data, individual Z-score value, as well as population-based values and made a final call determination. No calls were changed. All trisomy calls in this data set were correct. The performance in the training set was: sensitivity>99% (95% confidence interval: 96% to 100%) and specificity>99.9% (95% confidence interval: 99% to 100%), see FIG. 95 for confusion matrices and dot plots of the common trisomies tested. The sex chromosome aneuploidies calls are summarized in FIGS. 97A-97C and depicted in FIG. 98. FIG. 97A shows sex chromosomal aneuploidies called based on the Y-chromosome and the X-chromosome representation. Female samples are expected to not show any Y-chromosome and their X-chromosome representation is expected to follow a normal distribution comparable to percentage representations for autosomes. Therefore, female samples and female sex chromosome aneuploidies can be classified using the X-chromosome representation. Male samples do have a Y-chromosome representation and show a strong correlation between decrease in X-chromosome representation and increase in Y-chromosome representation. This relationship can be measured and outliers can be determined. In the plot the green-shaded region represents normal females (XX), brown-shaded region shows normal males (XY), orange-shaded region represents Turner syndrome (X0), red-shaded represents XXX syndrome (XXX), the pink-shaded region represents and extra Y chromosome (XYY) and the purple-shaded region depicts an Klinefelter syndrome (XXY), the grey-shaded areas are indeterminant zones between the various sex chromosome classifications. FIG. 97B shows an X chromosome representation (xvec02) plot showing the increase in sequencing bin representation as the X chromosome dosage increases from X0 to XX to XXX in the fetus. If the mother is X0 or XXX the signal will be significantly amplified. FIG. 97C shows an X chromosome bin plot for an affected XXX sample which is maternal in origin vs a normal XX sample. The signal is normalized to 1.0 (FIG. 97C), the affected sample with an extra X chromosome clearly shows an increase to 1.5 as expected if the mother is XXX.

Out of the 664 samples which passed QC in the training set 655 had reference data available for SCA status. Of those 655 samples, a total 638 samples were called for SCA with 530 negative samples called correctly and 106 out of 108 sex chromosome aneuploidies were correctly identified. Two samples were correctly identified as a SCA, but misclassified compared to the reference karyotype (both samples are XYY but have been called XXY). Overall the SCA performance showed a sensitivity of >99% (95% CI: 97%-100%) and a specificity of >99.9% (95% CI: 99%-100%). The accuracy of the SCA calls was greater than 98% and the call rate 97.4%.

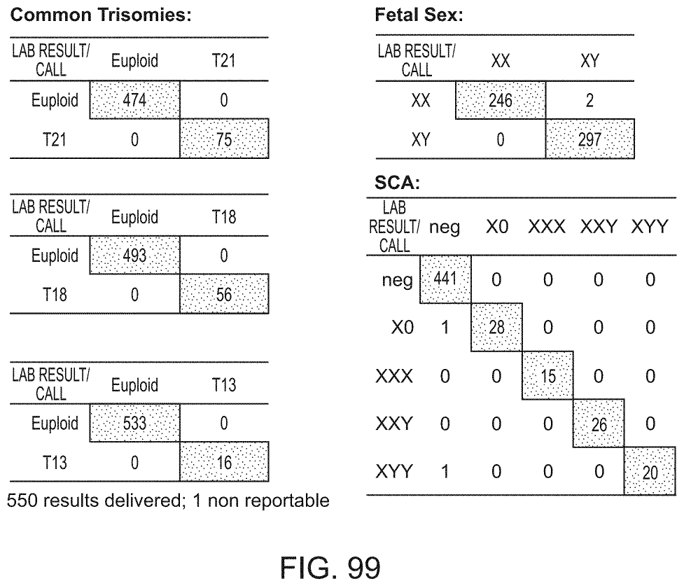

In the test set, sequencing yielded a median of 4.3 million sequenced ccfDNA fragments per sample. Seventeen samples (3.0%) produced less than 2 million sequenced ccfDNA fragments and failed QC. A second aliquot of plasma for these samples was processed, per standard clinical lab NIPT protocol, and all 17 subsequently passed QC. The automated calls were provided to a blinded independent reviewer. The independent reviewer examined available clinical data, individual Z-score value, as well as population-based values and made a final call determination. One sample was signed out as non-reportable and not re-tested. All calls in the test set were correct, resulting in a performance of: sensitivity>99% (95% confidence interval: 96% to 100%) and specificity>99.9% (95% confidence interval: 99% to 100%), see FIG. 99. The resulting sex chromosome aneuploidies calls from the test set of samples are depicted in FIG. 99. Out of all 550 samples a set of 534 had data available for SCA and a total 534 samples were called for SCA. Of the 534 samples, 441 out of 441 negative samples were called correctly and 89 out of 91 sex chromosome aneuploidies were correctly identified. Two samples were incorrectly identified as a euploid, one sample was a X0 sample and the other was a XYY. The overall sensitivity was 98% (95% CI: 92%-100%) with a specificity of >99.9% (95% CI: 99%-100%). The accuracy of the SCA calls was greater than 98% with a call rate greater than 99.9%.

The study cohort also contained designated multifetal samples (n=74) for which trisomies 21 (n=1), 18 (n=2) and 13 (n=1) were correctly detected (FIG. 98). In such cases the positive result only dictates that one of the fetuses is affected, it does not resolve the status of both.

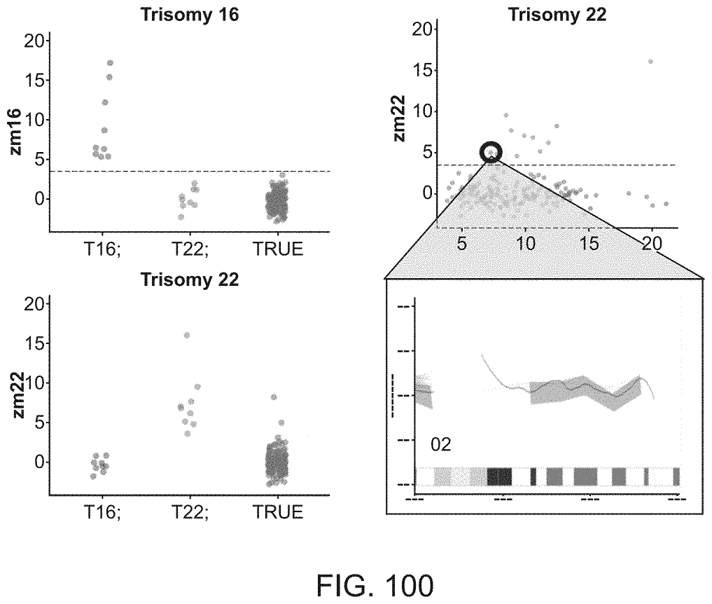

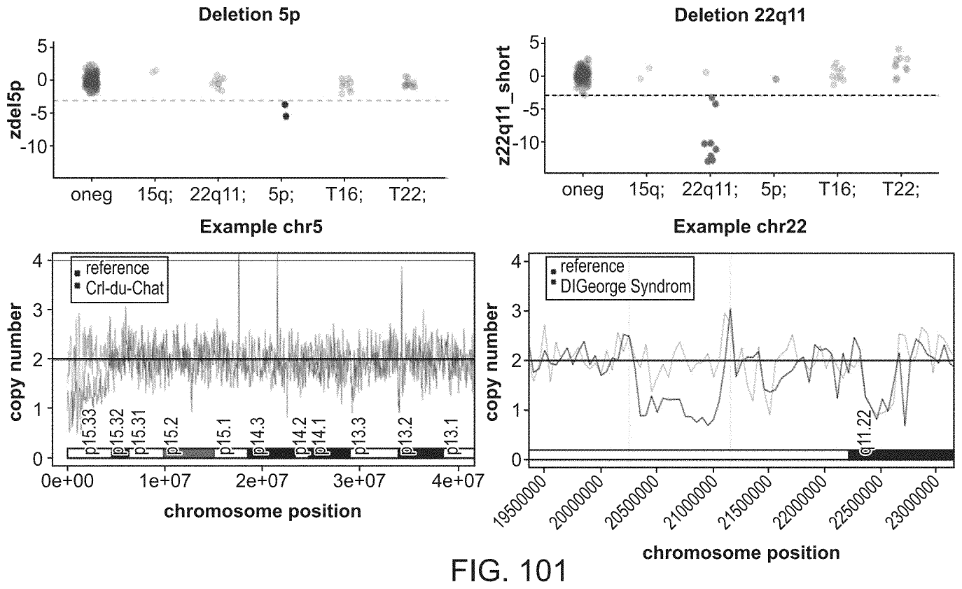

In addition to the common trisomies and sex chromosome aneuploidies the study cohort contained less common fetal trisomies in the form of trisomy 16 (n=9) and trisomy 22 (n=9) plus a subset of the more common fetal microdeletions including deletion 22q11 or DiGeorge syndrome (n=8), deletion 5p or Cri du Chat syndrome (n=2) and deletion 15q (n=2). All of the trisomy 16 and 22 cases were correctly identified plus 1 additional sample designated as euploid in the reference data. The high Z-score and genome plot indicate that one it was a true trisomy 22. Another sample with an elevated Z-score had a large copy number variation on the short arm of chromosome 22 and was designated as euploid at the time data review (see FIG. 100). The fetal microdeletions included in the study cohort were all correctly identified (see examples in FIG. 101).

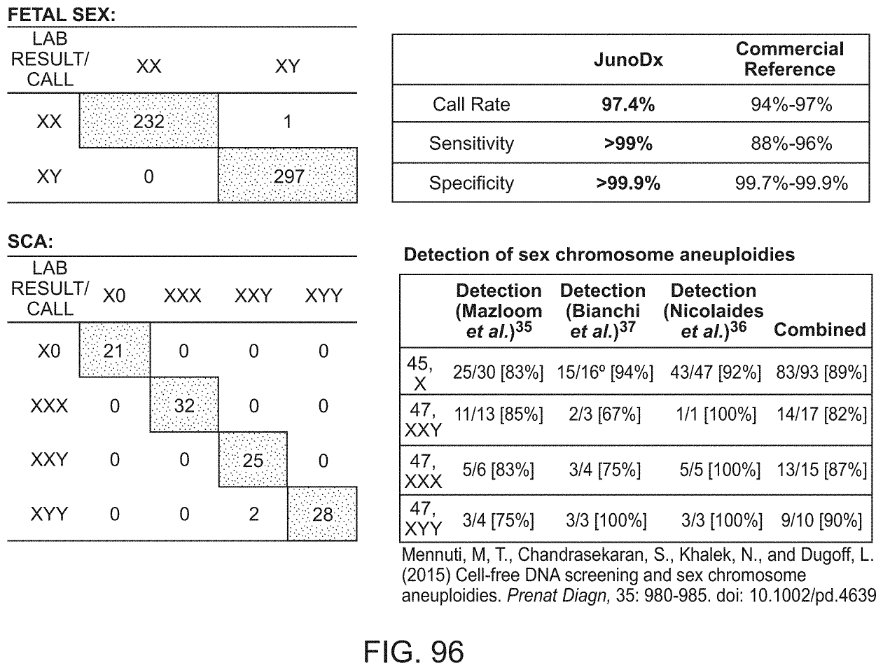

The data in this study clearly demonstrates that the ultra-low input NIPT process is extremely robust and reproducible at generating NIPT data suitable for accurate classification of clinical samples equal to that of high volume NIPT. The process used for this study uses significantly lower input plasma volumes, ccfDNA extraction volumes, whole genome library generation volumes and sequencing coverage. The study was composed of over 1200 maternal blood samples collected from pregnant females between 9-33 weeks with a median gestational age of 12 weeks. All samples had previously undergone NIPT in a licensed clinical laboratory and that data was used as the reference to determine performance of the ultra-low input NIPT process. The samples were randomly divided into a training set (n=671) and a test set (n=550) in order to first optimize the process (training set) and then test it in a blinded fashion as would be done in a clinical NIPT lab (test set). The results from both sets were impressive given the large size and number of affected cases of the study cohort. FIG. 96 shows the breakdown of the training set classification performance for trisomies 21, 18, 13: sensitivity >99% (95% confidence interval: 96% to 100%), specificity>99.9% (95% confidence interval: 99% to 100%), the SCA performance was a sensitivity of >99% (95% CI: 97%-100%) and a specificity of >99.9% (95% CI: 99%-100%). FIG. 99 shows the breakdown of the test set classification performance for trisomies 21, 18, 13: sensitivity >99% (95% confidence interval: 96% to 100%) and specificity>99.9% (95% confidence interval: 99% to 100%), the SCA performance was sensitivity of 98% (95% CI: 92%-100%) with a specificity of >99.9% (95% CI: 99%-100%). Multifetal and less common fetal aneuploidy or deletion events including trisomy 16, trisomy 22, deletion 22q11, deletion 15q and deletion 5p were also included in the cohort and all were classified correctly (FIGS. 98-101).

### Example 6: Laboratory Testing of Plasma Separation Device Performance

### Background

Purified plasma is typically prepared from whole blood in clinical laboratory environments using a sequential centrifugal separation procedure intended to separate the liquid phase (plasma) from cellular components (red blood cells, immune cells, etc). This process is time consuming, requires significant operator effort, and is, potentially, prone to risk of sample contamination or exchange. The plasma separation device tested in this experiment is intended to replace the centrifuge process in the laboratory by collecting purified plasma via passage through a membrane contained in a device designed to capture cellular components of whole blood while permitting the liquid plasma compartment of the blood passage into a collection vessel.

Two mechanisms were used to estimate the efficiency of plasma separation and the quality of plasma collected using the plasma separation device. First, plasma volume, which is often expected to be approximately 40% of the input whole blood volume in most subjects, was measured using a single channel variable pipette. For example, a 75 μl blood sample may be expected to yield approximately 30 μl of purified plasma upon activation of the plasma separation device. Second, DNA content of the plasma, measured in terms of genomic copies per microliter of plasma using a multicopy gene locus quantitative PCR assay, was measured to determine the quality of the plasma collected. This approach assumes that increases in DNA yield relative to a paired sample prepared using the gold standard sequential centrifugation method are the consequence of lysis of cells during the plasma collection process, which is in turn reflective of poor performance of the plasma collection device.

Objectives:

This experiment sought to test the performance of a manufacturing lot of laminate membrane stacks in terms of efficiency of (a) efficiency of plasma separation and (b) quality of plasma collected using the metrics described above. In addition, limited tests of alternative lubricant (silicone) for the slider compression pad and of a fully integrated device prototype (with fully integrated plasma collection vessel) were performed.

Experimental Design:

This experiment tested a slider device with a new manufacturing lot of laminate stacks. The performance was evaluated with two donor individuals using a set of single-use plasma separation devices. Capillary blood collected from the donors was loaded either directly to the device/membrane assembly by dripping from a fingertip with a lancet cut or was collected in a K2EDTA microtainer tube and transferred to the device/membrane assembly in known volumes using an adjustable volume pipette. Membranes used in this experiment were designed to permit loading of a specific volume of whole blood, so when loading was performed directly from the fingertip, visual confirmation of a filled membrane was assumed to mean that more than the specific volume of whole blood was loaded.

All plasma separator devices were assembled prior testing with integrated membrane laminate stacks; only the compression pad was added individually prior to testing. Fingertips were lanced and droplets of blood collected in the plasma separation device, at which point the device was activated by sliding and the plasma recovered. In cases for which blood was added to the device in known quantities using a variable volume pipette, the blood was transferred directly from the microtainer tube to the membrane assembly for testing.

Plasma was separated from the remaining blood in the microtainer tube (>200 μl) using standard sequential centrifugation approaches. The microtainer tube was initially centrifuged at low speed for 20 minutes, after which the plasma supernatant was transferred to a new tube and centrifuged on high speed for 10 minutes. The resulting purified plasma was transferred to a new tube and used as a reference for each subject in the study. All sample processing occurred within four hours of the blood draw.

The volume of all resulting plasma samples were determined using a variable volume pipette and recorded as a result of the study. In this study, blood was either added to the laminate stack using a pipette, in which case input volume was a known quantity, or loaded by dripping the blood directly onto the membrane from the finger. In the former case, percent plasma yield was calculated as the fraction of plasma volume recovered to whole blood added. In the latter case, the membrane capacity was assumed to be a specific volume, so percent yield was determined as the volume of plasma recovered divided by the specific volume.

Ten microliters of each sample were aliquoted into secondary containers for cell-free DNA (cfDNA) extraction. cfDNA was eluted in 15 μl final volumes. In the event that less than 10 μl plasma was available, the remaining volume was filled with phosphate buffered saline solution prior to commencing the cfDNA extraction process. DNA quantification of resulting DNA was performed using a quantitative PCR assay targeting a multicopy gene located on the Y chromosome. The assay performed used custom TaqMan fluorescent probes and primer with results collected on a QuantStudio6 instrument. Quantity of DNA in each sample was determined in this assay relative to a standard curve generated from commercially available control genomic DNA. DNA content was reported as genomic complements of DNA per ul of plasma entering cfDNA extraction. As a means to compare to the gold standard method of centrifugation, cfDNA yield per μl plasma was compared between plasma separation device prepared samples and centrifugation prepared samples with the ratio of these two values representing the relative cfDNA yield for samples prepared using the separation device. This ratio was only performed for paired samples from the same donor at the same time to reduce the impact of biological variability in this comparison.

Results:

Device operation was relatively simple, although significant force was required to activate the device. Devices using silicone lubricant were easier to activate and produced more plasma than most other tests, although the resulting plasma was potentially more opaque than samples collected using devices without silicone lubricant.

Plasma Volume

Mean plasma yield was 14.3 μl and median plasma yield was 15.3 μl. This is consistent with approximate 20% plasma yield from blood inputs assuming an input of −75 μl whole blood permitted in the membrane assembly. Plasma yield was lower than desired in this experiment, both in terms of raw volume recovered and percentage of whole blood input. Detailed notes on plasma volume are shown in Table 8.

TABLE 8
Plasma volume

| Donor | Delivery | Plasma volume | Notes |
|---|---|---|---|
| M1 | Direct | 20.7 | Silicone lubricant used |
| M1 | Direct | 19.3 | Silicone lubricant used |
| M1 | Direct | 10.7 | |
| M3 | Direct | 16.2 | |
| M3 | Direct | 21 | |
| M3 | 50 μl pipette | 4.6 | |
| M3 | 75 μl pipette | 8 | |
| M3 | 125 μl pipette | 16 | |
| M3 | 75 μl pipette | 14.2 | |
| M3 | 125 μl pipette | 15 | |
| M3 | 50 μl pipette | 3.8 | |
| M3 | 150 μl pipette | 18.5 | |
| M3 | 150 μl pipette | 19 | |
| M3 | 85 μl pipette | 13.6 | |

DNA Yield

All DNA quantitation were compared to a paired donor sample prepared using centrifugation. Mean yield of CDNA was 450 (median 53) of the centrifuge reference. This reflects a very high quality of plasma recovered with minimal cellular contamination due to cell lysis. Virtually no DNA was recovered from tests using the silicone lubricant. The use of this lubricant inhibits downstream applications. Detailed notes on plasma quality measures are shown in Table 9.

TABLE 9
Plasma quality

| | | Copies/ | Genomic copies | Fraction of |
|---|---|---|---|---|
| Donor | Delivery | μl | per μl plasma | centrifuge |
| M1 | Direct | 0.07 | 5.7% | Silicone lubricant |
| | | | | used |
| M1 | Direct | 0 | 0.0% | Silicone lubricant |
| | | | | used |
| M1 | Direct | 0.56 | 24.2% | |
| M3 | Direct | 1.09 | 47.0% | |
| M3 | Direct | 1.33 | 57.3% | |
| M3 | 50 μl pipette | 1.22 | 52.9% | |
| M3 | 75 μl pipette | 1.56 | 67.5% | |
| M3 | 125 μl pipette | 1.34 | 57.9% | |
| M3 | 75 μl pipette | 1.24 | 53.7% | |
| M3 | 125 μl pipette | 1.32 | 57.1% | |
| M3 | 50 μl pipette | 0.43 | 18.8% | |
| M3 | 150 μl pipette | 1.85 | 80.0% | |
| M3 | 150 μl pipette | 1.26 | 54.4% | |
| M3 | 85 μl pipette | 1.08 | 46.8% | |

Test of Fully Integrated Device

A single sample was collected using a fully integrated device prototype containing a molded cavity for plasma collection with blood dripped directly onto the membrane prior to activation. Similar to the above experiments, a paired sample was collected and prepared via sequential centrifugation as a comparison. In this experiment, 38 μl plasma was recovered (approximately 50% of whole blood input volume) and the DNA content was 141% of the centrifuge reference. This may reflect a slight excess of positive pressure applied by the fully integrated device resulting in mild cell lysis, although visual inspection of the plasma suggested very high quality plasma recovery.

Conclusions:

Slider assemblies were very robust, with few assembly or manufacturing defects observed.

Silicone lubricants are unlikely to work for this application.

Plasma volume recovered was lower than desired in this experiment, suggesting the need for marginally greater positive pressure applied to improve volume recovery.

DNA yields were consistent across all tests, suggesting stable performance of the multiple assemblies in this lot.

### Example 7: Laboratory Testing of Plasma Separation Device Performance

Objectives:

This experiment sought to test different configurations of the plasma separation device in terms of (a) efficiency of plasma separation and (b) quality of plasma collected using the metrics described above. In addition, observational notes were employed to evaluate usability of the membrane laminate stack construction and device design with a specific intent to evaluate the ability of blood to freely enter the membrane laminate stack upon loading and prior to device activation.

Experimental Design:

This experiment tested three different laminate stack designs: three membrane laminate stack designs were tested: standard circular 4 mm inlet, standard circular 4 mm inlet with peripheral vent holes, and 4 mm inlet with cross-cut inlet. In addition, three different mechanisms for preparing the membrane material were tested: die-cut membrane, laser cut membrane cut without blue shipping backing, and laser cut with blue shipping backing still attached. Each of the above conditions were tested with three donor individuals, using an array of devices. Capillary blood collected from the donors was loaded either directly to the device/membrane assembly by dripping from a fingertip with a lancet cut or was collected in a K2EDTA microtainer tube and transferred to the device/membrane assembly in known volumes using an adjustable volume pipette. Membranes used in this experiment were designed to permit loading of a certain volume of whole blood, so when loading was performed directly from the fingertip, visual confirmation of a filled membrane was assumed to mean that more than the certain volume of whole blood was loaded.

Plasma separator devices were assembled during testing with membrane laminate stacks as called for in the experiment, typically 2-3 assemblies at a time. Fingertips were lanced and droplets of blood collected in the plasma separation device, at which point the device was activated by sliding and the plasma recovered. In cases for which blood was added to the device in known quantities using a variable volume pipette, the blood was transferred directly from the microtainer tube to the membrane assembly for testing.

Plasma was separated from the remaining blood in the microtainer tube (>200 μl) using standard sequential centrifugation approaches. The microtainer tube was initially centrifuged at low speed for 20 minutes, after which the plasma supernatant was transferred to a new tube and centrifuged on high speed for 10 minutes. The resulting purified plasma was transferred to a new tube and used as a reference for each subject in the study. All sample processing occurred within four hours of the blood draw.

The volume of all resulting plasma samples were determined using a variable volume pipette and recorded as a result of the study. In this study, blood was either added to the laminate stack using a pipette, in which case input volume was a known quantity, or loaded by dripping the blood directly onto the membrane from the finger. In the former case, percent plasma yield was calculated as the fraction of plasma volume recovered to whole blood added. In the latter case, the membrane capacity was assumed to be a specific volume, so percent yield was determined as the volume of plasma recovered divided by the specific volume.

Ten microliters of each sample were aliquoted into secondary containers for cell-free DNA (cfDNA) extraction using a MagMax cfDNA kit according to manufacturer instructions scaled to low volume inputs. cfDNA was eluted in 15 μl final volumes. In the event that less than 10 μl plasma was available, the remaining volume was filled with phosphate buffered saline solution prior to commencing the cfDNA extraction process. DNA quantification of resulting DNA was performed using a quantitative PCR assay targeting a multicopy gene located on the Y chromosome. The assay performed used custom TaqMan fluorescent probes and primer with results collected on a QuantStudio6 instrument. Quantity of DNA in each sample was determined in this assay relative to a standard curve generated from commercially available control genomic DNA. DNA content was reported as genomic complements of DNA per ul of plasma entering cfDNA extraction. As a means to compare to the gold standard method of centrifugation, cfDNA yield per μl plasma was compared between plasma separation device prepared samples and centrifugation prepared samples with the ratio of these two values representing the relative cfDNA yield for samples prepared using the separation device. This ratio was only performed for paired samples from the same donor at the same time to reduce the impact of biological variability in this comparison.

Results:

All operators in this experiment noted that blood entered the membrane much more quickly and easily when using the 4 mm inlet with cross-cut inlet in the membrane laminate stack. This was true of all three users, who were likely to have significantly different blood behavior in this domain based on preliminary testing.

Plasma Volume:

Mean plasma yield was 30 and median plasma yield was 35 μL. This is consistent with a 40-45% plasma yield from blood inputs assuming an input of −75 μl whole blood permitted in the membrane assembly. Detailed notes on plasma volume are shown in Table 10.

TABLE 10
Plasma volume

| | | | | Plasma |
|---|---|---|---|---|
| Donor | Input | Membrane | Device | volume |
| M3 | Direct | 4 mm inlet with vent holes | Phase 3 | 19 |
| M3 | Direct | 4 mm inlet with cross vent | Phase 3 | 40 |
| M3 | Direct | 4 mm inlet, die cut | Phase 3 | 31 |
| M1 | Direct | 4 mm inlet, laser cut no backing | Phase 3 | 43 |
| M1 | Direct | 4 mm inlet, die cut | Phase 3 | 31 |
| M3 | Direct | 4 mm inlet, die cut | Square | 27 |
| M2 | Direct | 4 mm inlet with cross vent | Phase 3 | 20 |
| M2 | Direct | 4 mm inlet, die cut | Phase 3 | 10 |
| M3 | Pipet 75 | 4 mm inlet, die cut | Square | 38 |
| | μl | | | |
| M3 | Pipet 75 | 4 mm inlet, laser cut with | Square | 46 |
| | μl | backing | | |
| M3 | Pipet 75 | 4 mm inlet, laser cut no backing | Square | 36 |
| | μl | | | |
| M3 | Pipet 75 | 4 mm inlet, laser cut no backing | Square | 34 |
| | μl | | | |
| M3 | Pipet 75 | 4 mm inlet, laser cut with | Phase3 | 37 |
| | μl | backing | | |
| M3 | Pipet 75 | 4 mm inlet, laser cut with | Phase3 | 36 |
| | μl | backing | | |
| M3 | Pipet 75 | 4 mm inlet, die cut | Phase3 | 11 |
| | μl | | | |

DNA Yield:

All DNA quantitation were compared to a paired donor sample prepared using centrifugation. Mean yield of DNA was 55% (median 57%) of the centrifuge reference. This is slightly lower than might be expected, but also likely reflects a very high quality of plasma recovered with minimal cellular contamination due to cell lysis. Detailed notes on plasma quality measures are shown in Table 11.

TABLE 11
Plasma quality measures

| | | | | Genomic | Fraction |
|---|---|---|---|---|---|
| | | | | copies per μl | of |
| Donor | Input | Membrane | Device | plasma | centrifuge |
| M3 | Direct | 4 mm inlet with vent holes | Phase 3 | 1.57 | 55.8% |
| M3 | Direct | 4 mm inlet with cross vent | Phase 3 | 1.44 | 50.9% |
| M3 | Direct | 4 mm inlet, die cut | Phase 3 | 1.86 | 66.1% |
| M1 | Direct | 4 mm inlet, laser | Phase 3 | 0.52 | 40.1% |
| | | cut no backing | | | |
| M1 | Direct | 4 mm inlet, die cut | Phase 3 | 0.75 | 58.1% |
| M1 | Direct | 4 mm inlet, die cut | Square | 2.38 | 84.4% |
| M2 | Direct | 4 mm inlet with cross vent | Phase 3 | 0.55 | 44.0% |
| M2 | Direct | 4 mm inlet, die cut | Phase 3 | 0.54 | 43.1% |
| M3 | Pipet 75 | 4 mm inlet, die cut | Square | 0.73 | 25.8% |
| | μl | | | | |
| M3 | Pipet 75 | 4 mm inlet, laser | Square | 1.63 | 57.8% |
| | μl | cut with backing | | | |
| M3 | Pipet 75 | 4 mm inlet, laser | Square | 1.75 | 62.2% |
| | μl | cut no backing | | | |
| M3 | Pipet 75 | 4 mm inlet, laser | Square | 1.63 | 57.8% |
| | μl | cut no backing | | | |
| M3 | Pipet 75 | 4 mm inlet, laser cut | Phase3 | 1.76 | 62.4% |
| | μl | with backing | | | |
| M3 | Pipet 75 | 4 mm inlet, laser cut | Phase3 | 1.21 | 42.8% |
| | μl | with backing | | | |
| M3 | Pipet 75 | 4 mm inlet, die cut | Phase3 | 2.07 | 73.5% |
| | μl | | | | |

Conclusions:

Membrane laminate assemblies with the cross cut inlet permitted blood to enter the membrane much easier for all users.

Plasma recovery volumes are in line with expectations and are sufficient for most downstream applications.

Quality of plasma recovered appears to be generally very high, with little to no evidence of cell lysis having taken place during device activation. However, less DNA than expected was recovered in this experiment, suggesting that there may be a slight loss of DNA in the device as part of the collection process.

Recovery volumes and DNA yield were fairly consistent across all tests, suggesting similar performance of the different laminate assemblies.

1. A method comprising:
(a) providing or obtaining a blood sample obtained from an individual;
(b) applying a positive pressure to a starting volume of the blood sample such that the blood sample is pushed or forced into a filter or a membrane, wherein the starting volume of the blood sample is not more than about 1 milliliter (mL);
(c) filtering the blood sample through the filter or the membrane to separate plasma from the blood sample, and
(d) collecting the plasma in a collection vessel, wherein a volume of the plasma is greater than about 25% of the starting volume of the blood sample.

2. The method of claim 1, wherein the volume of the plasma is greater than about 30%, greater than about 35%, or greater than about 40% of the starting volume of the blood sample.

3. The method of claim 1, wherein at least about 60%, at least about 65%, at least about 70%, at least about 75%, at least about 80%, at least about 85%, at least about 90%, or greater, of the total amount of plasma present in the blood sample is collected.

4. The method of claim 1, wherein the plasma comprises cell-free nucleic acids.

5. The method of claim 4, wherein the method results in an enrichment of cell-free nucleic acids.

6. The method of claim 1, wherein the starting volume of the blood sample is not more than about 500 microliters (μL), not more than about 250 μL, not more than about 150 μL, not more than about 100 μL, not more than about 80 μL, not more than about 60 μL, not more than about 50 μL, not more than about 40 μL, or not more than about 25 μL.

7. The method of claim 1, wherein cellular nucleic acids are reduced in the plasma, or wherein the plasma is substantially free of cellular nucleic acids.

8. The method of claim 1, wherein cells, cell fragments, microvesicles, or any combination thereof, are reduced in the plasma.

9. The method of claim 1, wherein the blood sample is or comprises capillary blood.

10. The method of claim 1, wherein the blood sample is or comprises whole blood or one or more blood components.

11. The method of claim 1, wherein a volume of plasma collected in (d) is greater than a volume of plasma collected using an equivalent method using negative pressure or vacuum, such as at least about 10%, at least about 25%, at least about 50%, at least about 75%, at least about 100%, or greater than a volume of plasma collected using an equivalent method using negative pressure or vacuum.

12. The method of claim 1, wherein the positive pressure is of an amount of less than about 4 psi, an amount of 4 psi to about 11 psi, or an amount greater than about 11 psi.

13. The method of claim 1, wherein the method is performed in 1 minute or less.

14. The method of claim 1, wherein the method does not result in substantial lysis or disruption of white blood cells.

15. The method of claim 1, wherein the positive pressure is selected such that hemolysis of red blood cells may occur, but white blood cells are not lysed or disrupted.

16. The method of claim 1, wherein the cell-free nucleic acids are deoxyribonucleic acid.

17. The method of claim 1, wherein the cell-free nucleic acids comprise cell-free nucleic acids from a tumor, cell-free nucleic acids from a fetus, or cell-free nucleic acids from a tissue or an organ.

18. The method of claim 1, wherein the cell-free nucleic acids comprise from about 10⁴ to about 10⁹ cell-free nucleic acid molecules.

19. The method of claim 1, wherein the blood sample is obtained from the individual by a finger prick.

20. The method of claim 1, wherein the method is performed on a point-of-care or point-of-need device.
